(19) United States
(12) Patent Application Publication
(10) Pub. No.: US 2001/0024798 A1
(43) Pub. Date: Sep. 27, 2001

(54) Biomimetic combinatorial synthesis

(76) Inventors: Matthew D. Shair, Boston, MA (US); Craig W. Lindsley, Ann Arbor, MI (US); Henry E. Pelish, Silver Spring, MD (US); Scott M. Sheehan, Needham, MA (US); Brian C. Goess, Cambridge, MA (US); Mark E. Layton, North Cambridge, MA (US); Lawrence K. Chan, Cambridge, MA (US); Chuo Chen, Cambridge, MA (US); Nicholas J. Westwood, Brookline, MA (US)

Correspondence Address: Choate, Hall & Stewart, Exchange Place, 53 State Street, Boston, MA, 02109, US

(21) Appl. No.: 09/838,760

(22) Filed: Apr. 19, 2001

Related U.S. Application Data
(63) Continuation of application No. 09/329,970, filed on Jun. 10, 1999, now abandoned.
(60) Provisional application No. 60/089,124, filed on Jun. 11, 1998.

(51) Int. Cl. G01N 33/53; C07D 241/04

(52) U.S. Cl. 435/7.1; 544/385

(57) ABSTRACT

The present invention provides biomimetic compounds and libraries thereof, as well as methods for their production. In general, the inventive method involves the selection of a desired biological synthetic pathway and mimics that synthetic pathway utilizing modern synthetic tools. The structures formed from this method are preferably generated in fewer than four steps. These scaffold structures can then be functionalized to yield biomimetic compounds and libraries of compounds. In preferred embodiments, biomimetic compounds and libraries are generated from an oxidative phenolic coupling reaction. In other particularly preferred embodiments, the compounds and libraries of compounds are generated from cascade reactions to yield bicyclo [n.3.1] ring systems, medium ring systems, and fused ring systems. In addition to compounds, libraries and methods for their production, the present invention also provides pharmaceutical compositions and methods and kits for determining one or more biological activities of the library members.

### PRIORITY INFORMATION

This application claims priority to the provisional application entitled “Biomimetic Combinatorial Synthesis”, Ser. No. 06/089,124, filed Jun. 11, 1998, the entire contents of which are incorporated herein by reference.

### BACKGROUND OF THE INVENTION

The identification of small organic molecules that affect specific biological functions is an endeavor that impacts both biology and medicine. Such molecules are useful as therapeutic agents and as probes of biological function. In but one example from the emerging field of chemical genetics, in which small molecules can be used to alter the function of biological molecules to which they bind, these molecules have been useful at elucidating signal transduction pathways by acting as chemical protein knockouts, thereby causing a loss of protein function. (Schreiber et al., J. Am. Chem. Soc., 1990, 112, 5583; Mitchison, Chem. and Biol., 1994, 1, 3) Additionally, due to the interaction of these small molecules with particular biological targets and their ability to affect specific biological function, they may also serve as candidates for the development of therapeutics.

Because it is difficult to predict which small molecules will interact with a biological target, intense efforts have been directed towards the generation of large numbers, or “libraries”, of small organic compounds. These libraries can then be linked to sensitive screens to identify the active molecules. In many cases, researchers have developed “biased” libraries, in which all members share a particular characteristic, such as an ability to interact with a particular target ligand, or a characteristic structural feature designed to mimic a particular aspect of a class of natural compounds. For example, a number of libraries have been designed to mimic one or more features of natural peptides. Such “peptidomimetic” libraries include phthalimido libraries (WO 97/22594), thiophene libraries (WO 97/40034), benzodiazopene libraries (U.S. Pat. No. 5,288,514), libraries formed by the sequential reaction of dienes (WO 96/03424), thiazolidinone libraries, libraries of metathiazanones and their derivatives (U.S. Pat. No. 5,549,974), and azatide libraries (WO 97/35199) (for review of peptidomimetic technologies, see Gante, J., Angew. Chem. Int. Ed. Engl. 1994, 33, 1699-1720 and references cited therein).

Each of these libraries has provided solid phase synthetic strategies for compounds possessing specific core functionalities, but none achieves the complexity of structure found in natural products, or in other lead compounds prepared through traditional chemical synthetic routes. Complex natural products commonly contain several different functionalities and often are rich in stereochemical complexity. Such diversity and complexity is difficult to achieve if the synthesis is restricted to a specific class of compounds.

Recognizing the need for development of synthetic strategies that produce large numbers of complex molecules, Boger et al. (EP 0774 464) have recently developed a solution-phase synthetic strategy for producing a library of compounds based on a functionalizable template core, to which various reagents can be added. There remains a need, however, for the development of solid-phase strategies, where the more rapid production methods such as split-and-pool strategies can be employed to generate larger (>1,000,000), more complex libraries. Additional solution-phase strategies would, of course, also be valuable.

Because it is often the case that the synthesis of complex compounds, specifically natural products, requires performing sensitive multi-step reactions, in order to achieve this goal, it will be necessary to develop synthetic strategies that require fewer steps and that incorporate a wider range of synthetic reactions. One approach toward the achievement of this goal is the development of complex compounds and libraries of compounds utilizing biomimetic synthetic pathways. In this approach, a synthetic pathway employed in a biosynthesis may be mimicked by utilizing the tools of modem synthetic chemistry. A striking example of the initial development of this area of research includes the biosynthesis of a collection of related natural products called nonadrides, by Barton et al. (Barton et al., J. Chem. Soc. 1965, 1769; Barton et al., J. Chem. Soc., 1965, 1772) In vivo feeding experiments by Sutherland have provided evidence that one member of the nonadride family, glaucanic acid, is derived from homodimerization of two 9-carbon anhydride units, as shown in FIG. 1. This biosynthetic pathway was also mimicked by using triethylamine and the same anhydride units, thus producing a small amount of isoglaucanic acid. (Sutherland et al., J. Chem. Soc., Perkin Trans. I 1972, 2584) Another example is the efficient construction of polycyclic frameworks from cascade reactions in which multiple carbon-carbon bonds are formed in a single reaction. This concept is exemplified in nature by the biosynthesis of a number of complex structures including steroids via cation-olefin cyclizations and several alkaloids via oxidative phenolic couplings.

In order to achieve greater diversity and complexity in the synthesis of compounds and particularly libraries of compounds, it would be desirable to develop such methods by either utilizing or emulating the rapid and stereoselective pathways that nature uses in the synthesis of natural products for the efficient production of complex compounds and libraries of compounds. FIG. 2 depicts the preferred method of the present invention which involves the use of simple building blocks and subjecting them to biomimetic organic synthesis to generate libraries of natural product-like compounds. Any resultant novel complex libraries based on biomimetic pathways will certainly be useful in the quest to discover non-natural compounds having the binding affinities and specific characteristics of natural products, themselves the products of genetic recombination and natural selection.

### SUMMARY OF THE INVENTION

The present invention provides biomimetic compounds and libraries thereof, as well as methods for their production. According to the invention, biomimetic synthetic pathways are utilized or emulated for the construction of scaffold structures from which libraries of biomimetic compounds can be synthesized. The biomimetic compounds and libraries of compounds that are structurally reminiscent of natural products in that they contain multiple sites of functional diversity, contain multiple stereocenters and optionally possess certain structural features of existing natural products. Additionally, these biomimetic compounds may also be functionally reminiscent of natural products or other biomolecules.

In a preferred embodiment, biomimetic compounds and libraries of compounds are generated from an oxidative phenolic coupling reaction. In one example, a hetereo-β,β-phenolic coupling reaction is promoted between two electronically distinct phenols to yield a diversifiable tetracyclic scaffold. In another example, a homo-coupling reaction is promoted between two identical phenols to yield yet another diversifiable tetracyclic scaffold structure. In yet another example, an intramolecular coupling reaction is promoted to yield diversifiable scaffold structures.

In another preferred embodiment, the inventive biomimetic compounds and libraries of compounds are generated from a cascade reaction in which polycyclic scaffold structures and libraries of these structures are generated, such as the skeletons of natural products such as CP-225,917, CP-263,114, and taxol. The inventive method effects the vinylation of a cyclic β-keto ester to generate a 2-vinyl-2-methoxycycloalkanone, which upon reaction with a vinyl Grignard reagent, generates bicyclo[n.3.1] ring systems.

In yet another preferred embodiment, alternative ring systems can be generated from the vinylation of cyclic β-keto esters to generate a 2-vinyl-2-methoxycycloalkanone, subsequent reaction with a vinyl organometallic reagent, and trapping with an electrophile to yield the ring opened biomimetic structures. Furthermore, the present invention provides a method to generate fused ring structures from these ring opened biomimetic structures. In a preferred embodiment, a biomimetic ring opened structure is treated with base to effect a kinetic deprotonation and a transannular Michael addition, and subsequent trapping with an electrophile to generate a diversifiable biomimetic ring fused structure. These compounds may then be diversified to generate libraries of biomimetic ring fused compounds.

In addition to providing biomimetic compounds, libraries of compounds and methods for their production, the present invention also provides a novel Tentagel-based silicon linker and a method for its synthesis, that can be used in the preparation of solid support bound compounds and combinatorial libraries.

The present invention further provides a kit comprising a library of biomimetic compounds and reagents for determining one or more biological activities of the library members, and also methods for using a library of compounds for determining one or more biological activities of the library members. To give but one example, the biological activity can be determined by using a binding reagent, such as a direct reagent (e.g., a binding target) or an indirect reagent (e.g., a transcription based assay). In a preferred embodiment, the method for determining one or more biological activities of the inventive compounds comprises subjecting the inventive compounds to a biological target and determining a statistically significant change in a biochemical activity relative to the level of biochemical activity in the absence of the compound.

The present invention additionally provides pharmaceutical compositions. In a preferred embodiment, the pharmaceutical composition comprises one or more of the inventive compounds and a pharmaceutically acceptable carrier.

### DEFINITIONS

Before further description of the invention, certain terms employed in the specification, examples, and appended claims are collected and defined below:

“Biomimetic Combinatorial Synthesis”: As used herein, “biomimetic combinatorial synthesis” refers to the use of chemical synthetic strategies to recreate a biological reaction process in the solid phase or the solution phase to generate diversifiable biomimetic scaffold structures from which libraries of biomimetic compounds can be generated. It will be appreciated that the present invention encompasses those reaction processes that represent actual biological reaction pathways as well as those that emulate the efficiency and stereoselectivity so characteristic of biological reaction processes, while providing access to different reaction pathways. The inventive biomimetic combinatorial libraries preferably contain more than one million members.

“Biomimetic compound or structure”: As used herein, a “biomimetic compound or structure” is a compound that mimics structurally natural products found in nature, and contains multiple sites of functional diversity and multiple stereocenters. In preferred embodiments, the structures contain at least 4 sites of functional diversity and 5 stereocenters. These compounds may also optionally mimic the biological activity of natural products or other naturally occurring biomolecules. The term is used in the presently claimed invention to indicate that the novel complex combinatorial libraries being synthesized are reminiscent of the complex natural products found in nature that have been selected as promoters or inhibitors of particular cellular functions, in the sense that they contain multiple complex functionalities and contain multiple stereocenters.

“Linker unit”: The term “linker unit”, as used herein, refers to a molecule, or group of molecules, connecting a solid support and a combinatorial library member. The linker may be comprised of a single linking molecule, or may comprise a linking molecule and a spacer molecule.

“Identifier Tag”: The term “identifier tag” as used herein, refers to a means for recording a step in a series of reactions used in the synthesis of a chemical library. For the purposes of this application, the terms encoded chemical library and tagged chemical library both refer to libraries containing a means for recording each step in the reaction sequence for the synthesis of the chemical library.

“Electron Withdrawing Group”: The term “electron withdrawing group”, a term recognized in the art, refers, as used herein, to a tendency of a substituent to attract valence electrons from neighboring atoms, i.e., the substituent is electronegative with respect to neighboring atoms. A quantification of the level of electron-withdrawing capability is given by the Hammett sigma (ρ) constant. This well known constant is described in many references, for instance, J. March, Advanced Organic Chemistry, McGraw Hill Book Company, New York, (1992 4th edition), pp. 278-286. The Hammett constant values are generally positive for electron withdrawing groups. Examples of common electron withdrawing groups include, but are not limited to nitro, ketone, cyanide, chloride, and aldehyde.

“Electron donating group”: The term “electron donating group”, a term recognized in the art, refers, as used herein, to a tendency of a substituent to donate valence electrons to neighboring atoms, i.e., the neighboring atoms are electronegative with respect to the substituent. As discussed above, the Hammett sigma(a) constant provides a quantification of electron withdrawing capability, and Hammett constant values are generally negative for electron donating groups. Examples of common electron donating groups include, but are not limited to amino and methoxy.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 4-10 carbon atoms in their ring structure, and more preferably have 5, 6 or 7 carbons in the ring structure.

Moreover, the term “alkyl” (or “lower alkyl”) as used throughout the specification and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an ester, a formate, or a ketone), a thiocarbonyl (such as a thioester, a thioacetate, or a thioformate), an alkoxyl, a phosphoryl, a phosphonate, a phosphinate, anamino, an amido, an amidine, an imine, a cyano, a nitro, an azido, a sulfhydryl, an alkylthio, a sulfate, a sulfonate, sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of aminos, azidos, iminos, amidos, phosphoryls (including phosphonates and phosphinates), sulfonyls (including sulfates, sulfonamidos, sulfamoyls and sulfonates), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “arylkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents as described above, as for example, halogen, azide, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, sulfonamido, ketone, aldehyde, ester, a heterocyclyl, an aromatic or heteroaromatic moiety, —CF₃, —CN, or the like.

The terms “heterocyclyl” or “heterocyclic group” refer to 4- to 10-membered ringtructures, more preferably 4- to 7-membered rings, which ring structures include one to four heteroatoms. Heterocyclyl groups include, for example, pyrrolidine, oxolane, thiolane, imidazole, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

It will be noted that the structure of some of the compounds of this invention includes asymmetric carbon atoms. It is to be understood accordingly that the isomers arising from such asymmetry are included within the scope of this invention. Such isomers are obtained in substantially pure form by classical separation techniques and by sterically controlled synthesis.

The phrase “protecting group” as used herein, refers to a chemical group that reacts selectively with a desired functionality in good yield to give a derivative that is stable to further reactions for which protection is desired, can be selectively removed from the particular functionality that it protects to yield the desired functionality, and is removable in good yield by reagents compatible with the other functional group(s) generated during the reactions. Examples of such protecting groups include esters of carboxylic acids, ethers of alcohols and acetals and ketals of aldehydes and ketones.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described hereinabove. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

The term “solid support” refers to a material having a rigid or semi-rigid surface. Such materials will preferably take the form of small beads, pellets, disks, chips, dishes, multi-well plates, wafers or the like, although other forms may be used. In some embodiments, at least one surface of the substrate will be substantially flat. The term “surface” refers to any generally two-dimensional structure on a solid substrate and may have steps, ridges, kinks, terraces, and the like without ceasing to be a surface.

The term “polymeric support”, as used herein, refers to a soluble or insoluble polymer to which an amino acid or other chemical moiety can be covalently bonded by reaction with a functional group of the polymeric support. Many suitable polymeric supports are known, and include soluble polymers such as polyethylene glycols or polyvinyl alcohols, as well as insoluble polymers such as polystyrene resins. A suitable polymeric support includes functional groups such as those described below. A polymeric support is termed “soluble” if a polymer, or a polymer-supported compound, is soluble under the conditions employed. However, in general, a soluble polymer can be rendered insoluble under defined conditions. Accordingly, a polymeric support can be soluble under certain conditions and insoluble under other conditions.

### DESCRIPTION OF THE DRAWING

FIG. 1 depicts the biosynthesis of glaucanic acid.

FIG. 2 depicts the preferred biomimetic synthetic method of the present invention.

FIG. 3 depicts biosynthesis via oxidative phenolic coupling reactions.

FIG. 4 depicts benzoxanthenone natural products.

FIG. 5 depicts a biosynthetic proposal for the synthesis of carpanone.

FIG. 6 depicts one electron oxidants en route to carpanone.

FIG. 7 depicts hetero-β,β-phenolic couplings.

FIG. 8 depicts the preparation of phenolic substrates.

FIG. 9 depicts biomimetic heterodimerization via differential electronics.

FIG. 10 depicts dimerization with a specific linker.

FIG. 11 depicts specific heterodimerization reactions.

FIG. 12 depicts phenolic couplings with iodine (III).

FIG. 13 depicts the generalization of iodine (III) promoted homodimerizations.

FIG. 14 depicts iodine (III) promoted heterodimerizations.

FIG. 15 depicts a mechanism for iodine (III) promoted reactions.

FIG. 16 depicts some biogenetic aspects of phenol oxidation.

FIGS. 17A and 17B depict the retrosynthesis for crinine-like and galanthamine-like compounds and libraries of compounds.

FIGS. 18A and 18B depict the synthetic scheme en route to crinine-like and galanthamine-like compounds and libraries of compounds.

FIG. 19 depicts the interconversion of a galanthamine core and a crinine core.

FIG. 20 depicts the two-step stereospecific synthesis of the CP core structure using the triple-tandem reaction.

FIG. 21 depicts a rapid synthesis of a C-aryl taxane skeleton.

FIG. 22 depicts the rapid synthesis of bridgehead olefin-containing molecules.

FIG. 23 depicts the rapid assembly of complex bridgehead olefin-containing molecules.

FIG. 24 depicts the incorporation of aromatic rings in the triple-tandem cyclization.

FIG. 25 depicts the synthesis of medium-sized and fused ring systems.

FIG. 26 depicts the synthesis of biomimetic fused ring structures.

FIG. 27 depicts various functionalization reactions employed on the biomimetic scaffolds.

FIG. 28 depicts several examples of reactions performed on the biomimetic scaffolds.

FIG. 29 depicts one example of a biomimetic library design.

FIG. 30 depicts a general plan for biomimetic combinatorial synthesis.

FIG. 31 depicts a convergent synthesis plan.

FIG. 32 depicts linkage of the electron rich aromatic to the solid phase using a photolinker.

FIG. 33 depicts the solid phase heterocoupling employing photocleavage.

FIG. 34 depicts the linkage of the electron rich aromatic to the solid phase using a silicon linker.

FIG. 35 depicts the solid phase heterocoupling reaction employing a silicon linker.

FIG. 36 depicts the use of Tentagel-based silicon linker.

FIG. 37A and 37B depict solid phase heterodimerizations.

FIG. 38A and 38B depict solid phase functionalization of the hetero core.

FIG. 39A and 39B depict representative biomimetic library members.

FIG. 40 depicts representative biomimetic library members.

FIG. 41 depicts the concept of chemical genetics.

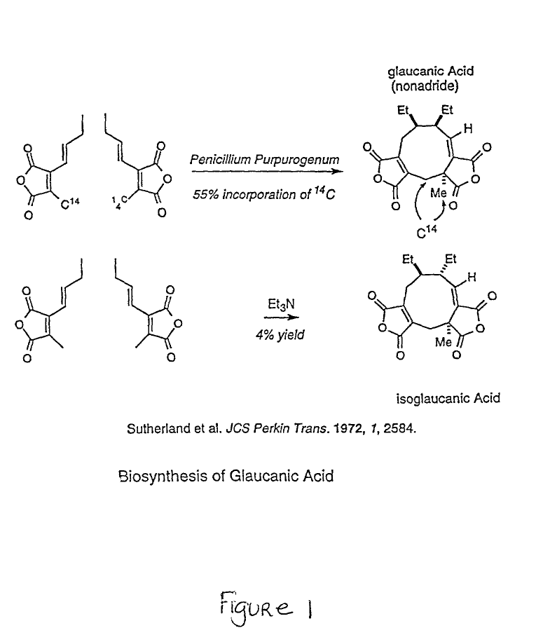

### DESCRIPTION OF CERTAIN PREFERRED EMBODIMENTS

One aspect of the present invention is the recognition that, in nature, elegant and powerful synthetic pathways are often employed to produce complex biological molecules. Chemical synthesis strategies can sometimes be designed to recreate a biological reaction process in a solid-phase (or solution phase) reaction process. For example, as discussed previously, Sutherland has reported that a single biological molecule, glaucanic acid, can be synthesized by a process that reproduces a biosynthetic pathway. Furthermore, in addition to the recreation of the exact biological reaction process, chemical synthesis strategies can also sometimes be designed to improve upon or change a biological reaction process, thus gaining efficient access to reaction pathways or stereospecificities previously unavailable in the natural process. The present invention for the first time provides natural and unnatural biomimetic synthesis strategies that allow the efficient production of large, diverse libraries of complex molecules that are structurally reminiscent of complex biological molecules.

In particular, the present invention utilizes biomimetic synthetic pathways for the construction of scaffold structures from which libraries of complex biomimetic compounds are synthesized. In certain embodiments, the tools of synthetic organic chemistry are utilized to improve and/or change the selectivity of traditional biomimetic reactions. As discussed, the present invention therefore encompasses not only the use of biological reaction pathways but also encompasses “non-natural” biological reaction pathways (with reactivity that might not be available in the “natural” system) designed to mimic biological pathways in their effeciency. The compounds represented in these libraries contain unprecedented complexity in comparison to other structures synthesized on the solid support. Furthermore, in some preferred embodiments of the invention, utilization of particular biomimetic synthetic strategies allows this complexity to be achieved in a one-step synthesis from easily synthesizable template structures.

As mentioned above, the present invention also contemplates the use of “modified” or “non-natural” biological reaction pathways. Thus, another aspect of the present invention is the recognition that it may often be desirable to obtain control over one or more competing reactions in a synthetic pathway. The present invention provides a method for achieving this control over one or more competing reactions involving the use of the solid phase in combination with a specific linker molecule to create specific microenvironments on the solid phase. In one preferred embodiment, the control of the reactivity of a specific reagent or reactant can be achieved. Alternatively or additionally, in other embodiments, the control of the microenvironment includes the control of the regioselectivity of reactions and/or the enantioselectivity of the reactions.

More generally, the present invention reproduces biosynthetic strategies in the context of controlled chemical syntheses. The particular biosynthetic reactions to be recreated are selected after consideration both of the potential for diversification of the structures they produce and for their experimental power and accessibility. In particular, factors relevant to the selection of a particular biosynthetic reaction include the amenability of the reaction to modern synthetic and solid phase reaction techniques, to include “natural” and “non-natural” reaction pathways, and the ability to produce complex molecules from easily obtainable starting materials in preferably one to four steps, to achieve functionalizable biomimetic scaffold structures from which isolable compounds and libraries of compounds can be generated.

Synthesis of Biomimetic Compounds via Oxidative Phenolic Coupling Reactions

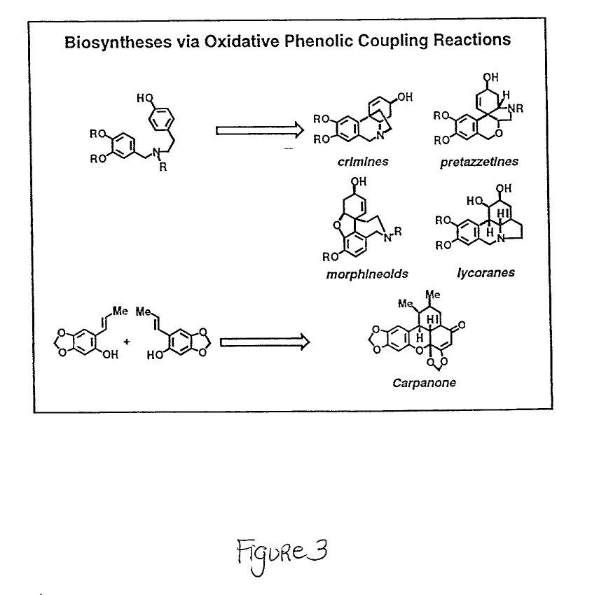

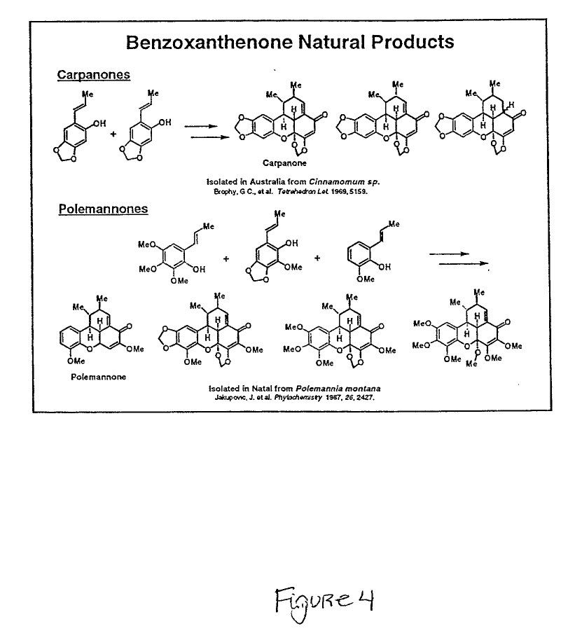

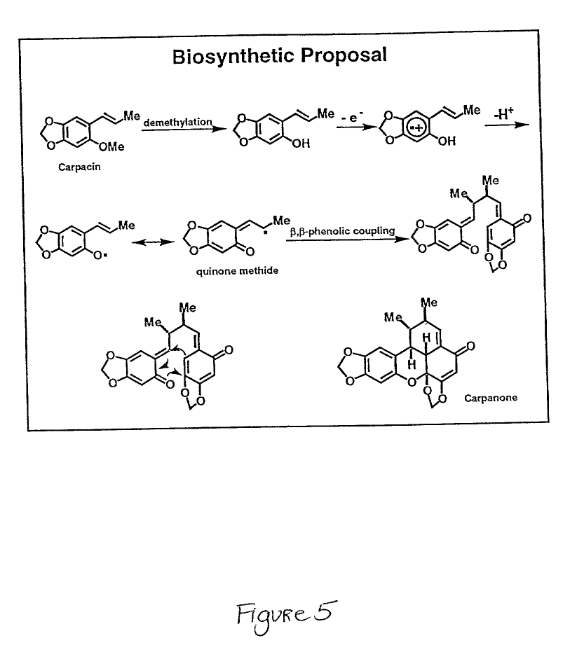

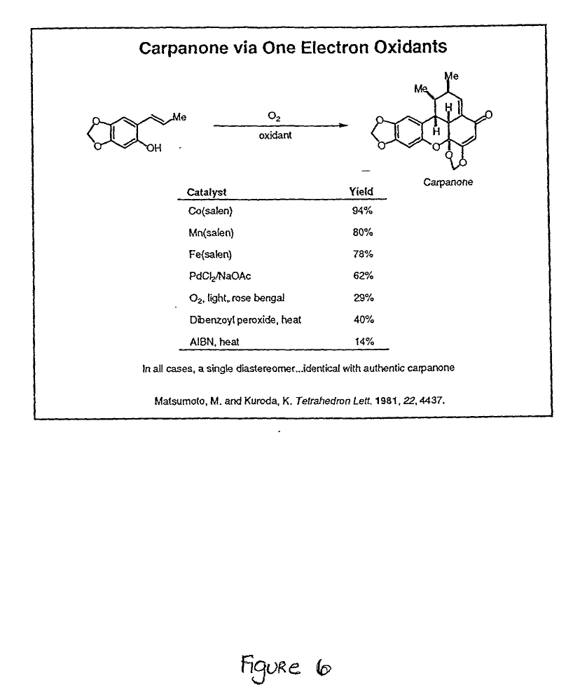

In one particularly preferred embodiment, the present invention employs an oxidative phenolic coupling reaction to achieve biomimetic scaffolds having core structures similar to several natural products. As shown in FIG. 3 oxidative phenolic coupling reactions are utilized to achieve the core structures of natural products such as crimines, pretazzetines, morphineoids, lycoranes, preseuomerin A and carpanone (a member of the benzoxanthenone class of natural products along with polemannones, as shown in FIG. 4). A biosynthetic proposal for the synthesis of one of these natural products, carpanone, is depicted in FIG. 5. Additionally, one synthesis of carpanone by Matsumoto and Kuroda from one electron oxidants is also shown in FIG. 6.

In the inventive method, reaction between two phenols is effected stereoselectively to achieve the scaffold structures utilized in the synthesis of the combinatorial libraries. The resulting scaffold structures are characterized by their rigidity, stereochemical and functional group complexity, and high density of functionality from which to generate highly diversified libraries. As one of ordinary skill in the art will realize, reaction with the phenols to yield the libraries of biomimetic compounds may be achieved via intermolecular or intramolecular oxidative coupling. Furthermore, for those reactions that occur intermolecularly, the same phenol can be utilized to effect the homodimerization reaction, or alternatively different phenols can be utilized to effect heterodimerization.

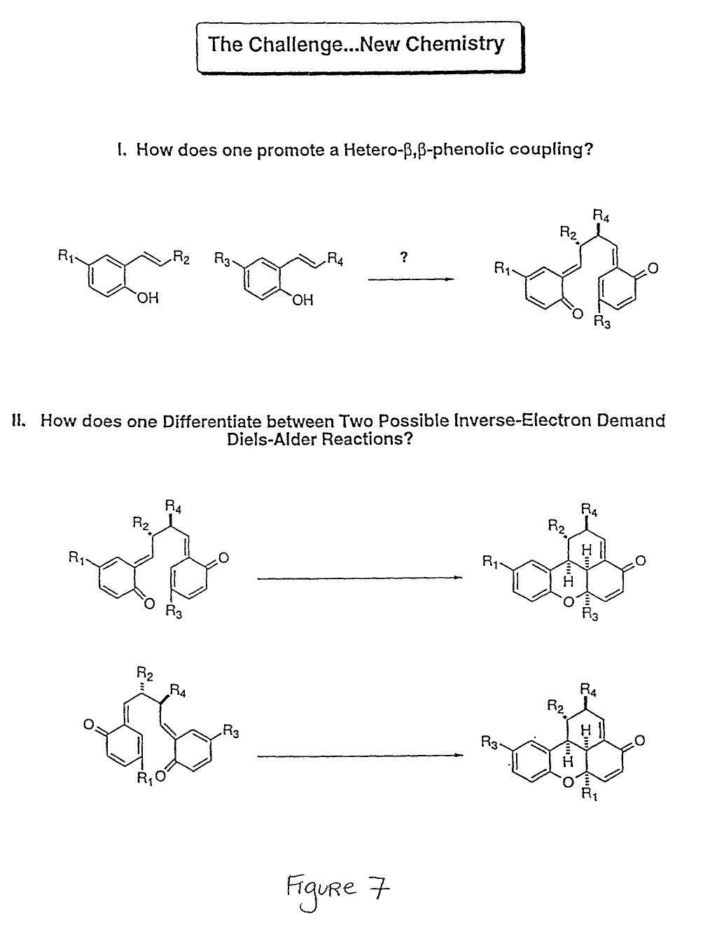

For example, Equation 1 below depicts the intermolecular oxidative coupling reaction, which may occur via homocoupling if one of the monomers reacts preferably with itself (that is, if, for the monomers depicted in Equation 1, R₂═R₇, R₃═R₈, R₄═R₉, R₅═R₁₀ and R₁═R₆) to yield a tetracycle, or may occur via heterocoupling if two different monomers, as shown in Equation 1 (where each monomer comprises different functional groups), react to yield another, diversifiable tetracycle in one step. Additionally, although only one of the possible inverse-electron demand Diels-Alder reactions is depicted, the presently claimed invention is intended to encompass all these products from both inverse-electron demand Diels-Alder reactions, as shown in FIG. 7. One of ordinary skill in the art will realize that functional group selection plays a role in the reaction pathway.

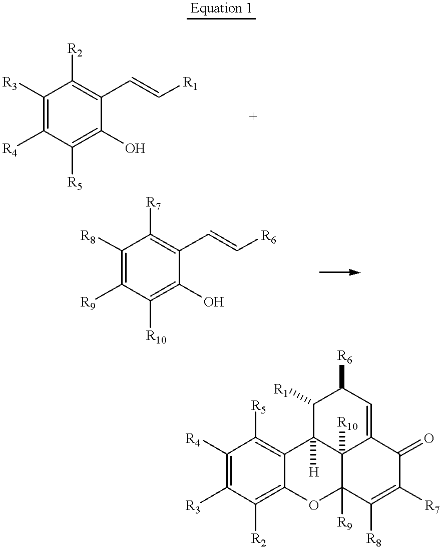

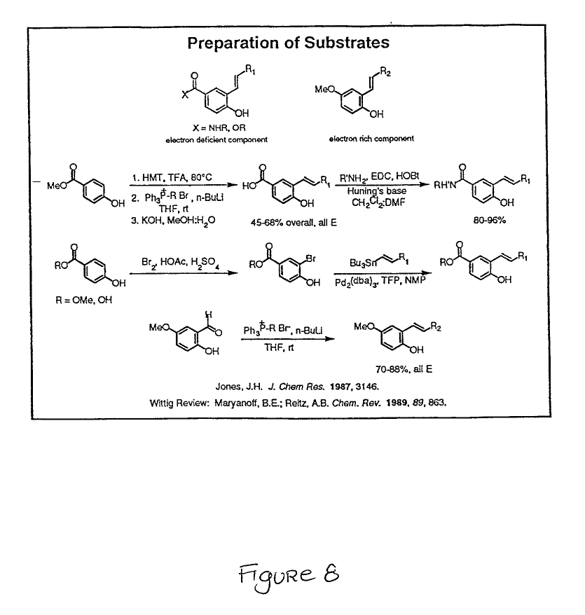

The phenols utilized in the inventive method are selected because they are readily available as shown in FIG. 8, and for their ability to react to generate a complex scaffold structure in one step, and have the general structure, as shown below (1):

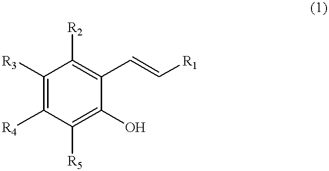

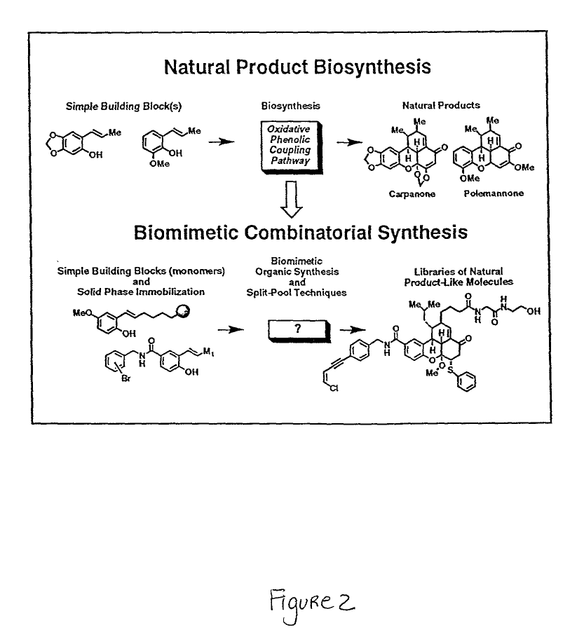

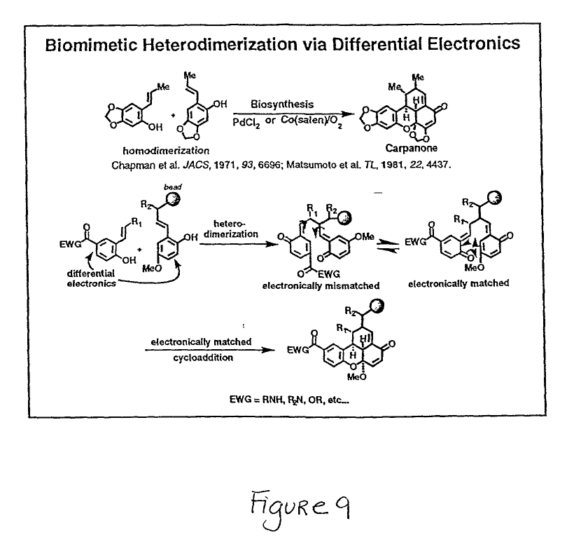

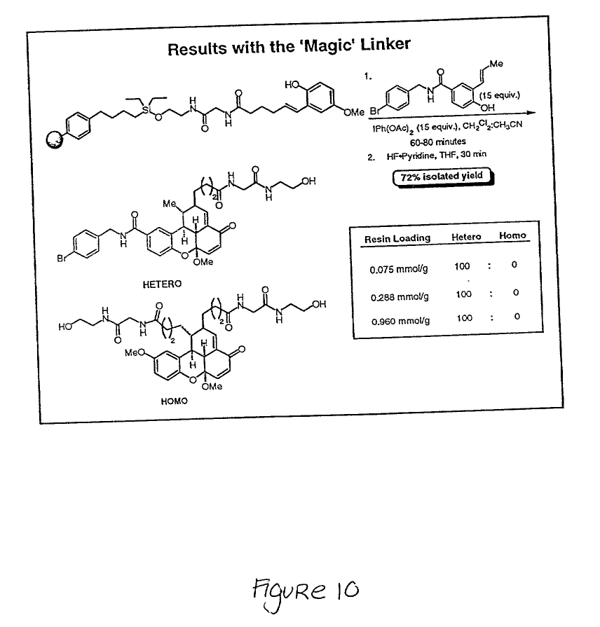

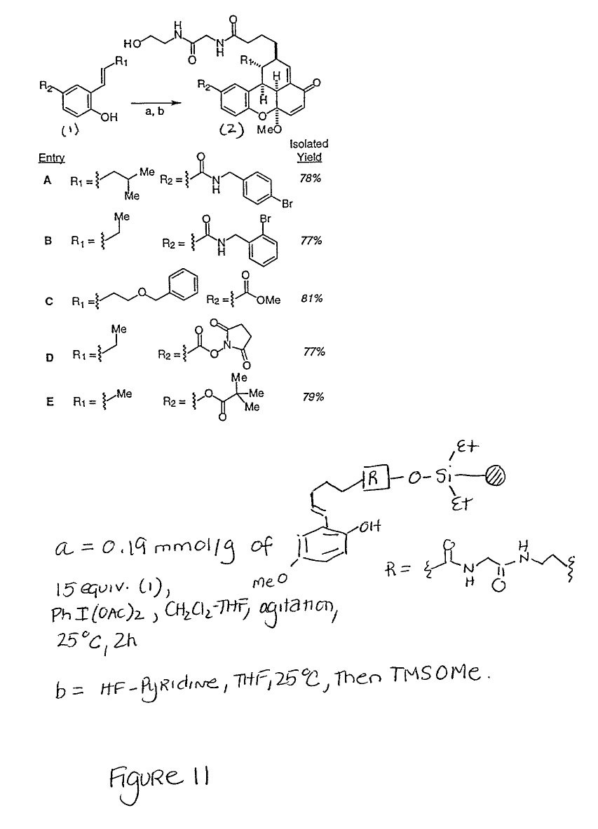

In order to effectively select the desired reaction to achieve a particular scaffold structure, electronic effects can be utilized and thus the functionality can be varied at each position on the aromatic ring (as represented by R₁-R₄ in FIG. 2) as shown in FIG. 9. For example, although homodimerization between any two molecules of either of the phenols utilized may occur, the pairing of an electron deficient phenol with an electron rich phenol, such as a methoxy derivative, will favorably select the heterodimerization product. R₃ for the electron rich phenol is most preferably, but is not limited to, hydroxy, methoxy, alkoxy, or amino. R₃ for the electron deficient phenol is most preferably, but is not limited to, carboalkoxy, or amide. In addition to selection of electron deficient and electron rich phenols as a method for controlling heterodimerization, the present invention, in another aspect also provides for control of specific microenvironments by utilizing specific solid phase linkers, and thus heterodimerization can also be promoted in this manner. In preferred embodiments, the use of amides along the chain linking the electron rich phenol to the solid phase is utilized. In particularly preferred embodiments, as shown in FIG. 10, a linking system comprising glycine and 2-aminoethanol is utilized. As also shown in FIG. 11, a series of electron deficient phenols bearing different groups in the R₁ position and different electron withdrawing groups in the R₂ position was successfully cyclized with resin-bound substrate. The solid phase biomimetic reaction tolerated several electron withdrawing groups at the R₂ position of (1), in FIG. 11, including amides, esters, activated esters and acylated phenols. In each case described, the tetracyclic adducts were obtained as a single compound resulting from complete electronic control in the inverse electron demand Diels-Alder cycloaddition and no sign of intrabead coupling. One of ordinary skill in the art will also realize that any electron donating or electron withdrawing group, respectively, could be utilized, with the limitation that these groups do not interfere with the desired oxidative phenolic coupling reaction.

Additionally, R₁, R₂, R₃, R₄, R₅, R₆, R₇, R₈, R₉, and R₁₀, may each independently be selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hydroxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocycl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, or any derivative incorporating phosphorous; or any of R₂, R₃, R₄, and R₅ taken together form a carbocycle or heterocycle having from 3 to 10 atoms in the ring, with the proviso that the functionalities selected must promote a desired oxidative phenolic coupling reaction (homodimerization or heterodimerization) as discussed above.

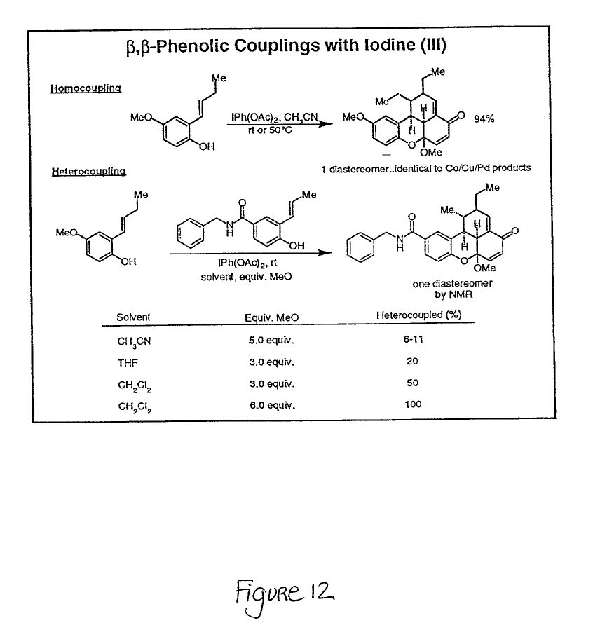

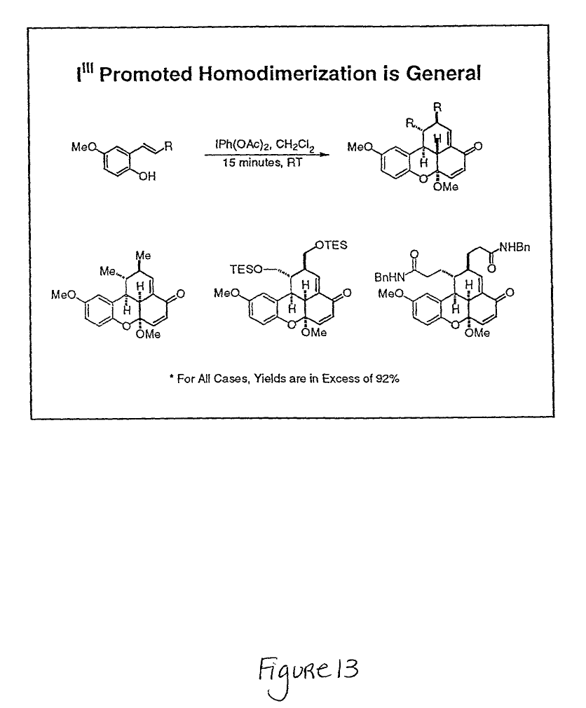

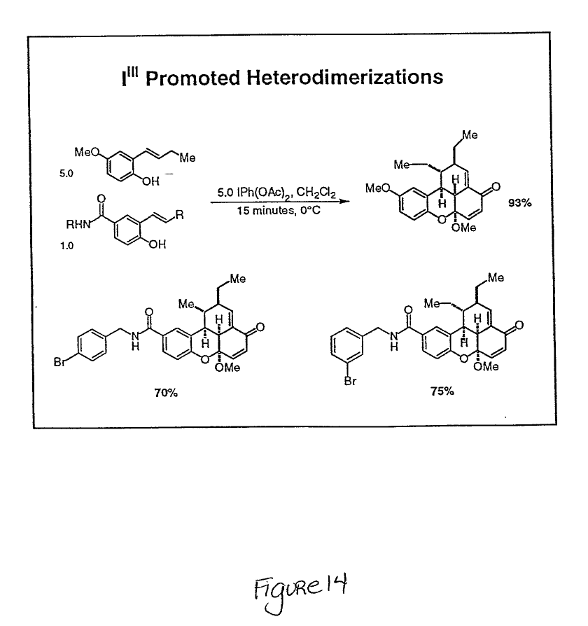

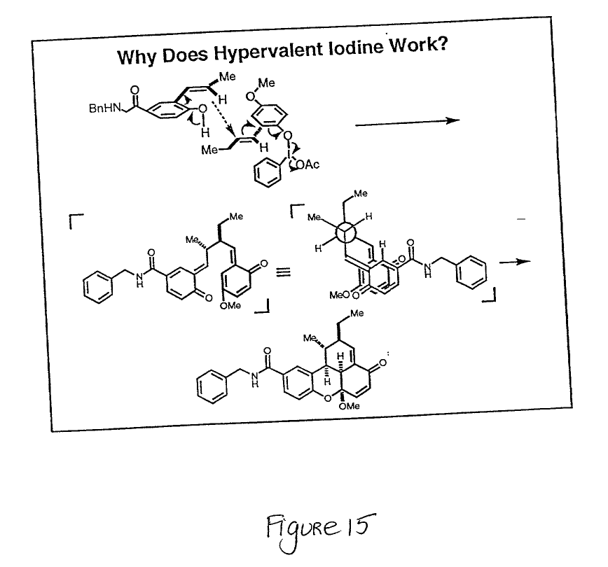

In one particularly preferred embodiment, promotion of the oxidative phenolic coupling to yield the inventive scaffolds and libraries can be effected utilizing an iodine (III) reagent such as IPh(OAc)₂. The advantages of utilizing an iodine(III) reagent are the increased yield and the stereoselectivity of the reaction. Notably, the products of heterodimerization and homodimerization both yield one diastereomer in good yield, as shown in FIG. 12. For homodimerization, the iodine(III) promoted reaction is general as shown in FIG. 13. Additionally, FIG. 14 depicts the heterodimerization of two phenols utilizing IPh(OAc)₂ at room temperature. Furthermore, the utilization of a chiral iodine(III) reagent, such as a chiral iodine(III) binap reagent, will most preferably yield a specific enantiomer from the reaction between the phenols. FIG. 15 depicts the mechanism of the β,β-phenolic oxidative coupling with hypervalent iodine.

In other embodiments of the presently claimed invention, the oxidative phenolic coupling may be promoted using one electron oxidants including but not limited to Co(salen), Mn(salen), Fe(salen), PdCl₂/NaOAc, O₂/light, dibenzoyl peroxide/heat, and AIBN/heat, to yield the inventive libraries. A subsequent Diels-Alder reaction between the coupled reagents yields the scaffold structure in good yield and stereoselectively.

Alternatively, in an another particularly preferred embodiment of the presently claimed invention, an intramolecular reaction may be effected to generate a diverse array of scaffold structures from which complex natural product-like combinatorial libraries may be generated. In one example, a tetracyclic structure is generated from an intramolecular reaction, as shown generally in Equation 2.

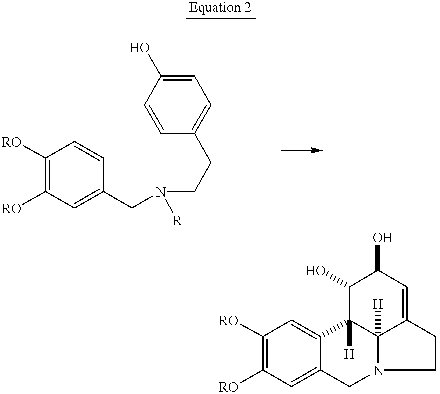

The inventive intramolecular method can be generalized to encompass any linked phenols comprising the following structure (2) below:

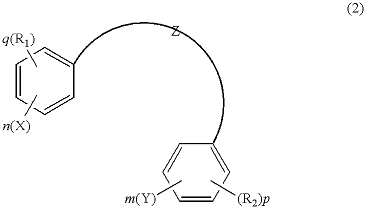

wherein R₁ or R₂, for each occurrence, each independently comprise a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocycyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, or any derivative incorporating phosphorous, and q and p are preferably independently 0-4. X and Y preferably independently comprise hydroxy, thio, or amino, and n and m are preferably independently 1-5. Furthermore, any combination of the functionalities on the phenolic substrates may also comprise a heterocyclic or carbocyclic structure.

As one of ordinary skill in the art will realize, the various reactive sites present in the structure above will yield a variety of different biomimietic compounds. In particular, the substituents present in the linked ring systems will affect the reaction pathway for the generation of scaffold structures. For example, when X═OH, the positioning of the hydroxyl group determines whether a para-para, ortho-para, para ortho, or ortho-ortho nucleophilic substitution reaction will occur. As an example of the diversity and utility of this reaction, from a single structure four different natural product cores can be synthesized such as those found in crimines, pretazzetines, morphineoids, and lycoranes, as shown in FIG. 3, or more specifically lycorine, crinine or galanthamine, as shown in FIG. 16. Additionally, depicted (3) below is a general scheme depicting the possible reactions for linked phenols.

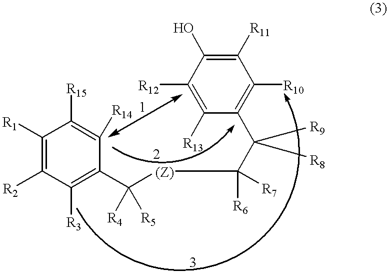

1. Para-ortho Coupling:

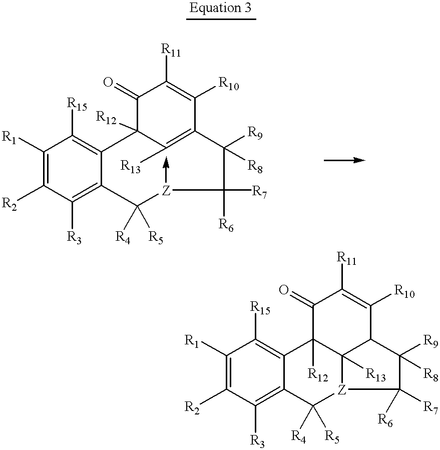

2. Para-para Coupling:

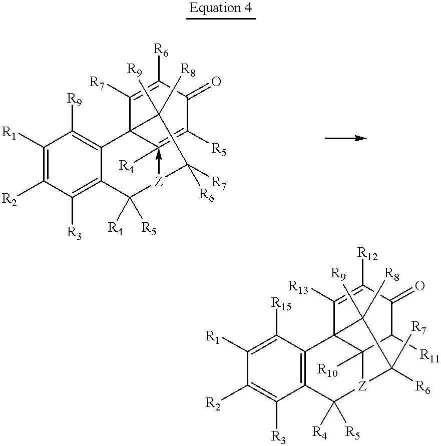

3. Ortho-para Coupling:

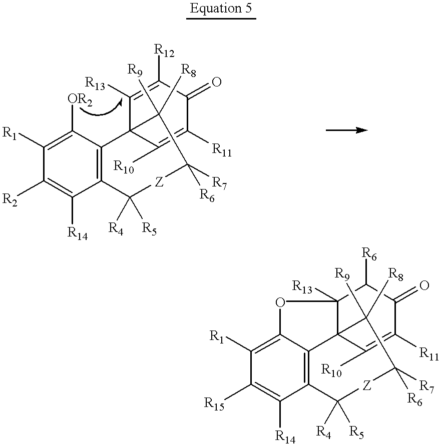

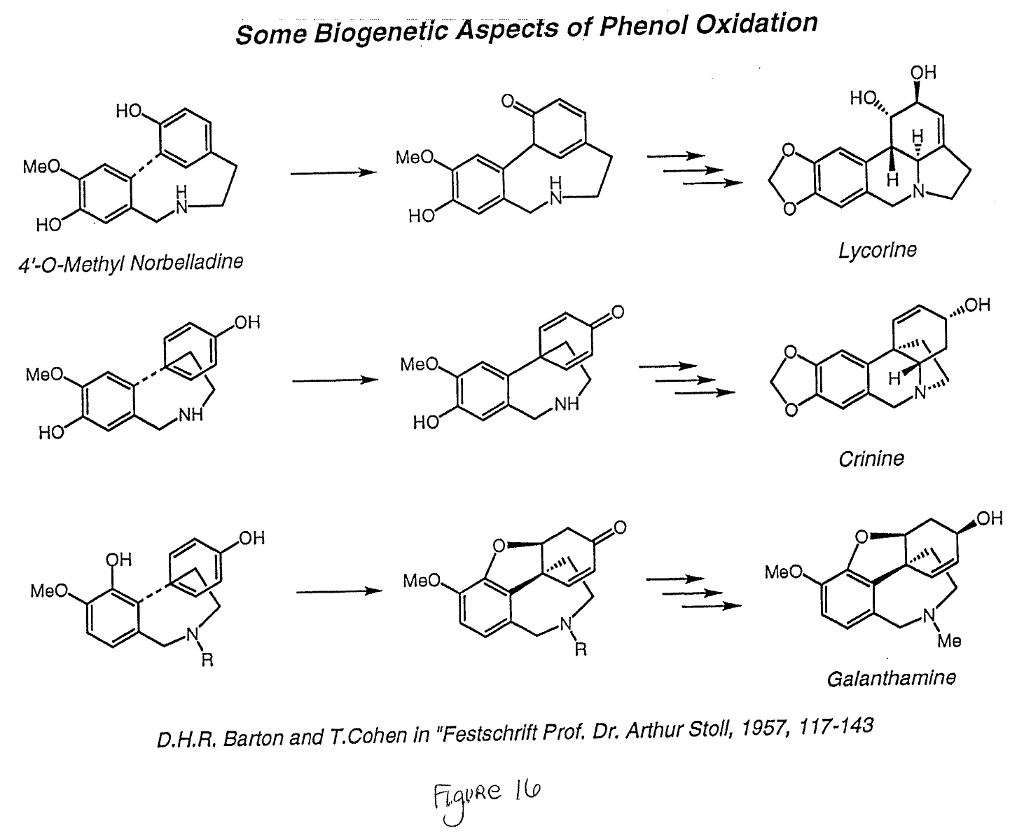

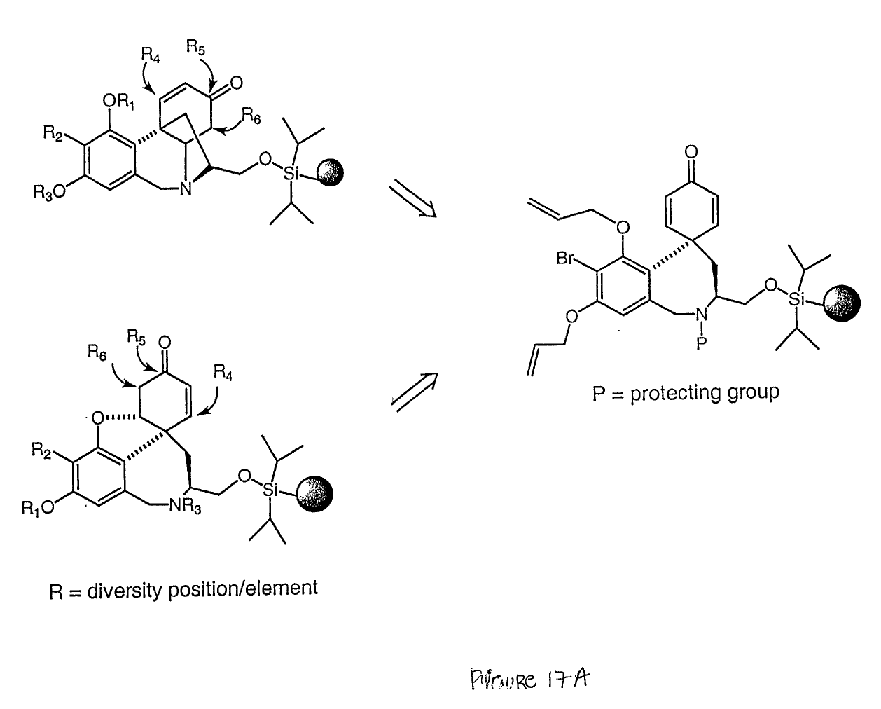

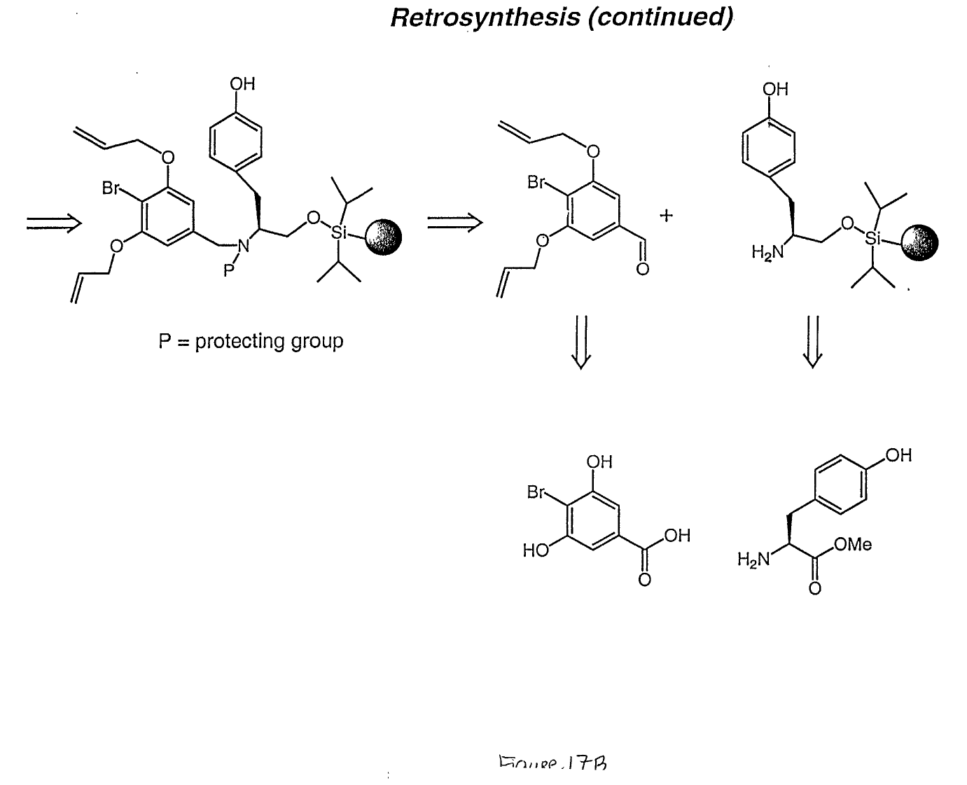

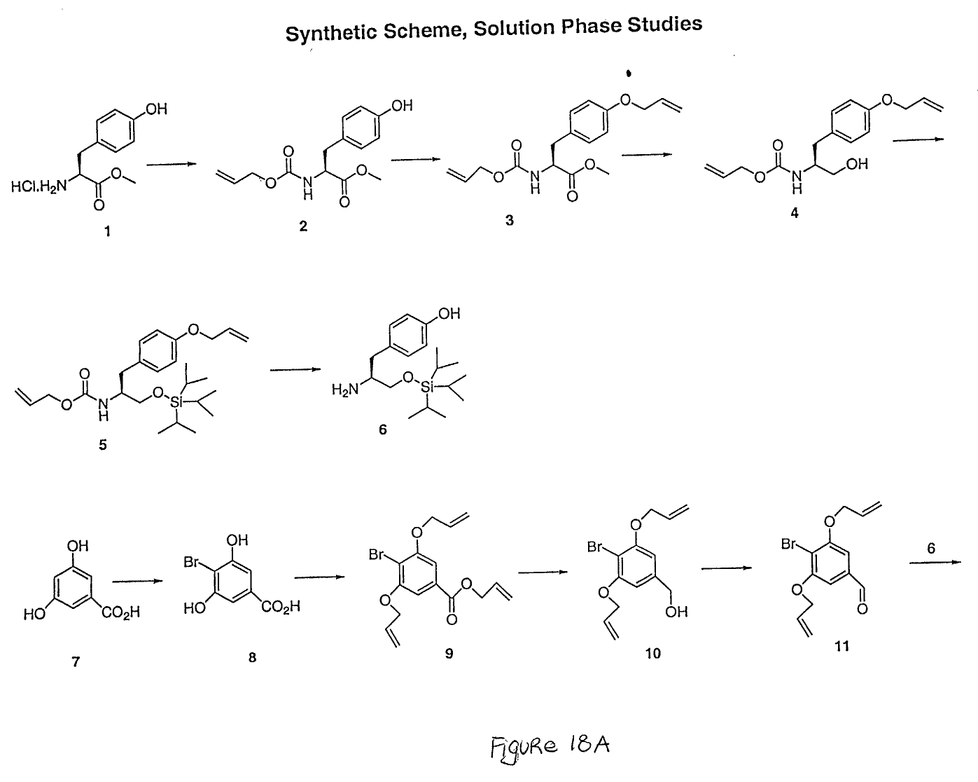

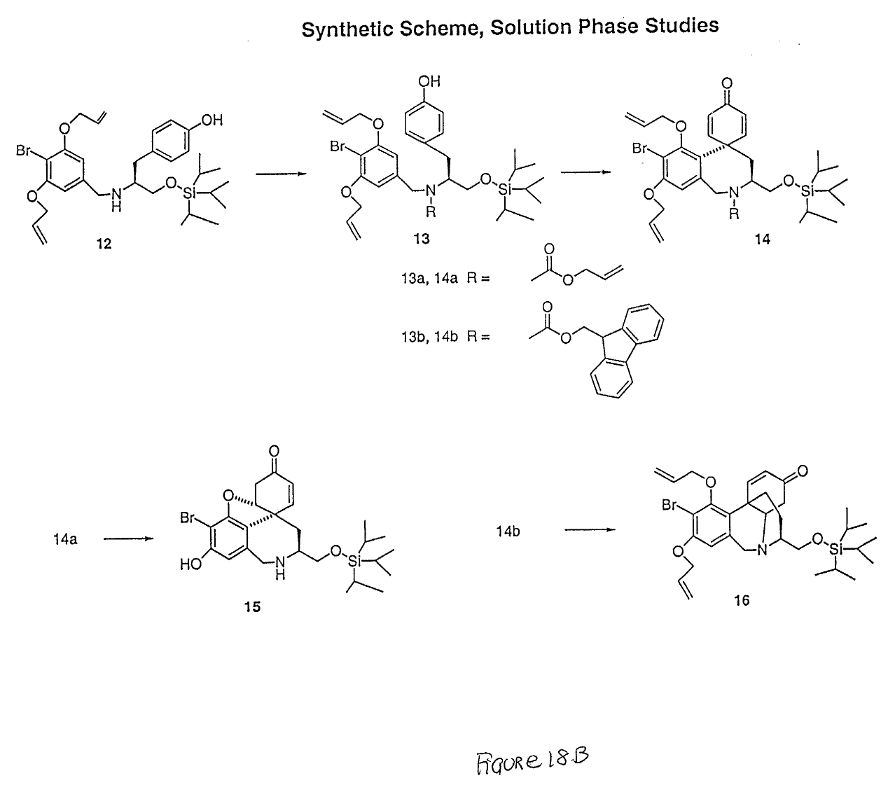

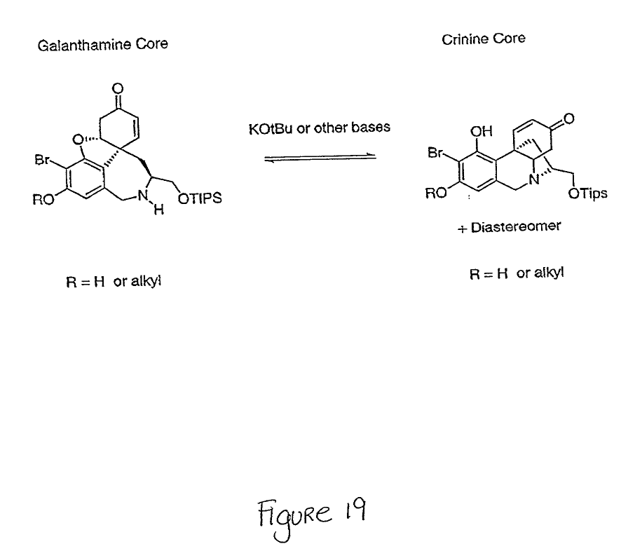

In one exemplary embodiment of the present invention, galanthamine-like and crinine-like compounds and libraries of compounds are prepared. FIG. 17A and 17B depict the retrosynthesis of support-bound galanthamine-like and crinine-like core structures, corresponding to the para-para and ortho-para reaction products as shown in Equations 4 and 5 above, having various latent sites of functionality for diversification. More particularly, FIGS. 18A and 18B depict the synthesis of the inventive compounds which are described in detail in the examples section below. The present invention also recognizes the efficiency of obtaining multiple libraries of compounds from one core structure. Thus, in an exemplary embodiment, as shown in FIG. 19, either the galanthamine-like core or the crinine-like core structures can be transformed into the other structure using bases, including but not limited to, KOtBu.

Synthesis of Biomimetic Compounds via Cascade Reactions

In another particularly preferred embodiment, the present invention employs a cascade reaction involving a tandem vinyl organometallic addition, an anionic oxy-Cope rearrangement, and a transannular cyclization. The concept of the utilization of cascade reactions is exemplified in nature by the biosynthesis of a number of complex structures. For example, a particularly powerful cascade reaction sequence is macrocyclization followed by transannular cyclization. This concept has been utilized by nature during the biosynthesis of a large number of structurally diverse terpenes from a small set of terpenoid building blocks. Taxol and longifolene are two examples which involve cation-initiated macrocyclization followed by transannular cyclization. The inventive method mimics the efficiency of these biosynthetic pathways by linking facile sigmatropic rearrangements with additional cyclization reactions. In an exemplary embodiment, this is achieved by the synthesis of a vinyl stannane from a substituted alkyne, vinylation of a cyclic β-keto ester to generate a 2-vinyl-2-methoxycycloalkanone, and subsequent reaction with a Grignard reagent to generate bicyclo [n.3.1] ring systems, as shown in Equation 6 below.

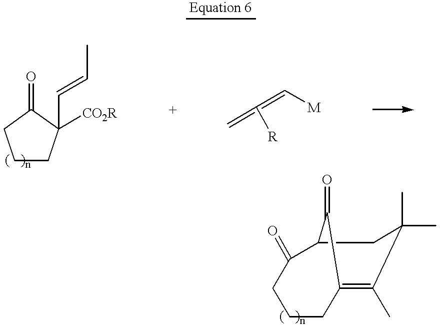

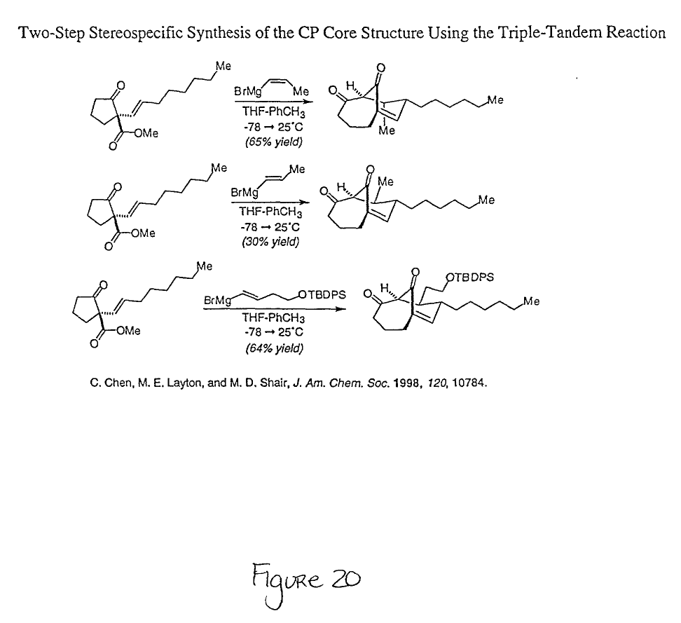

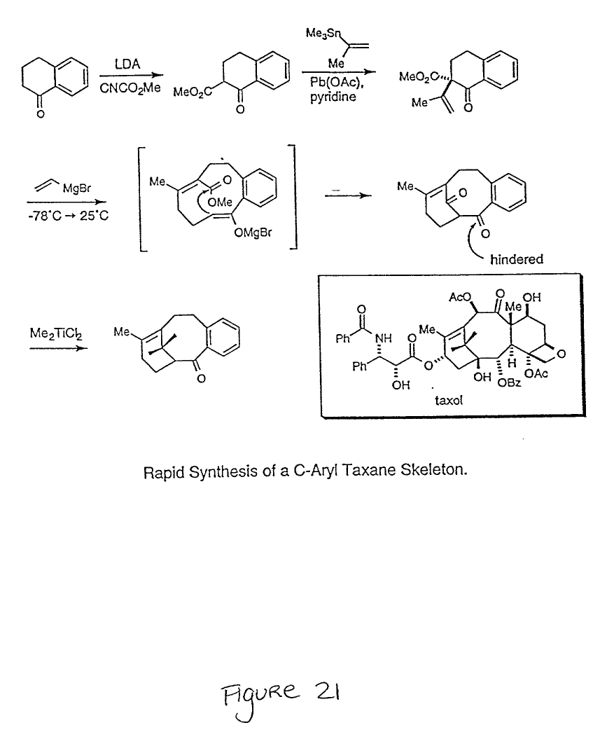

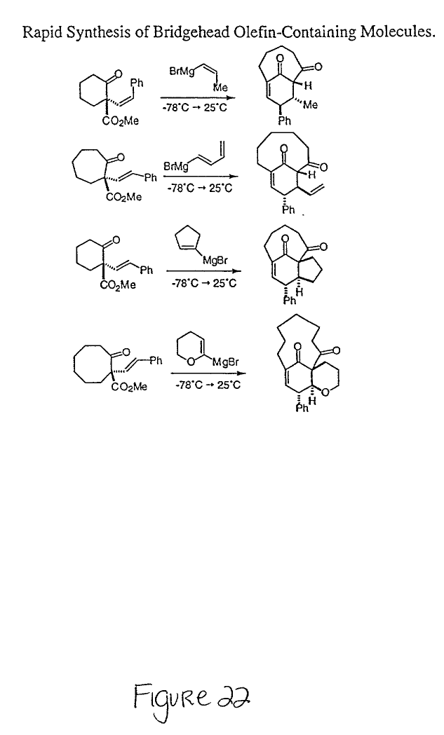

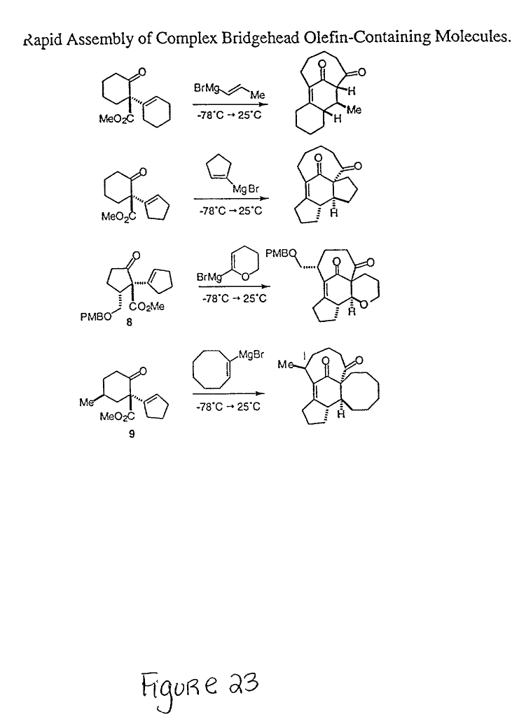

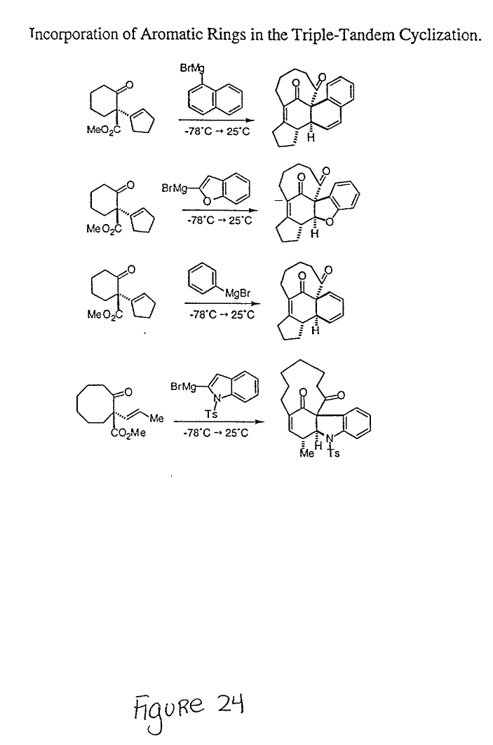

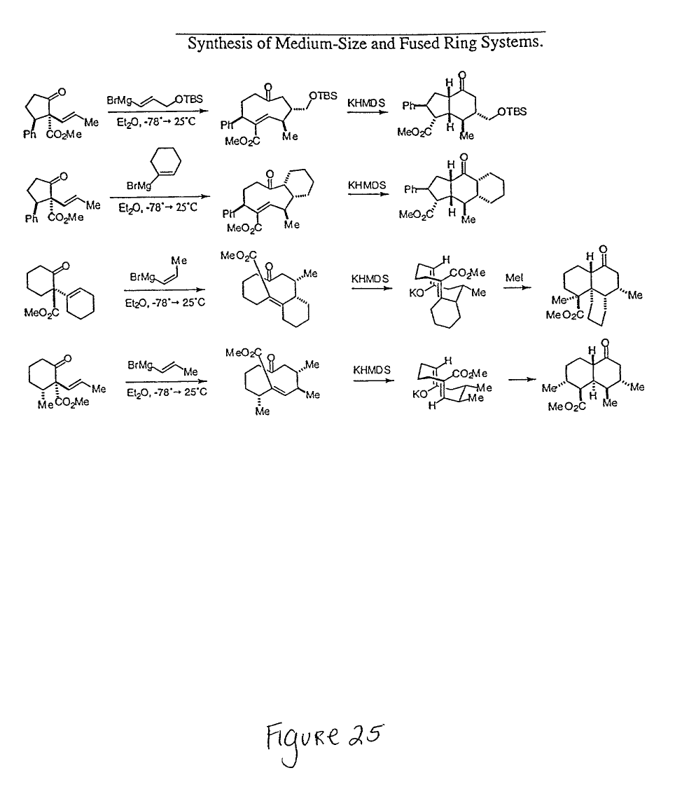

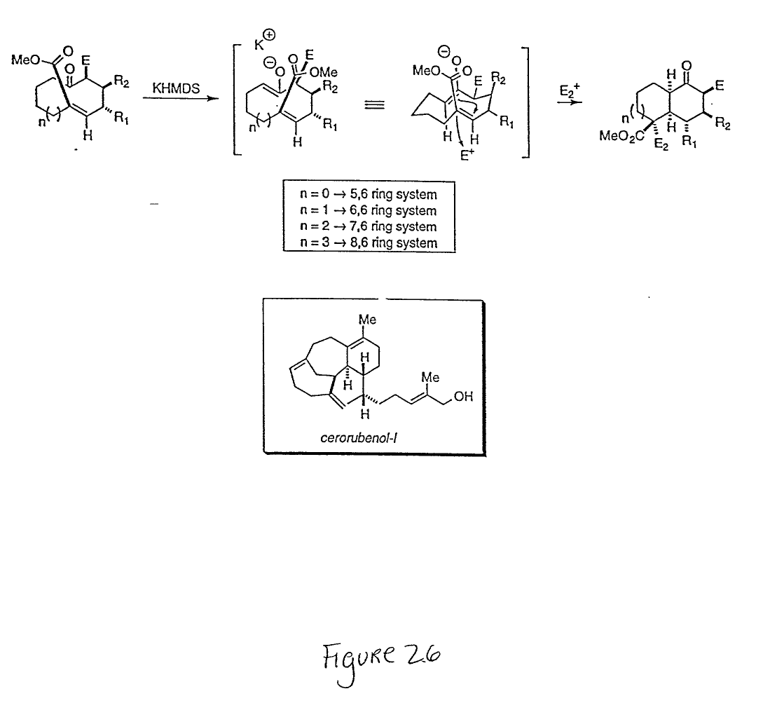

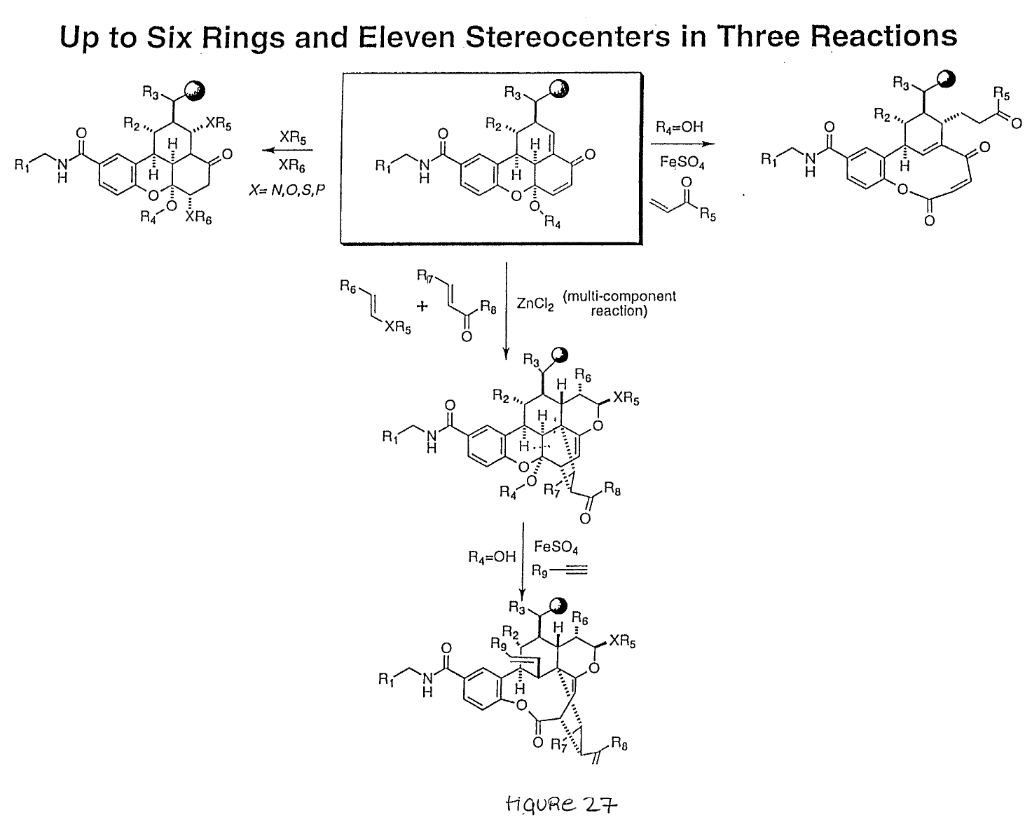

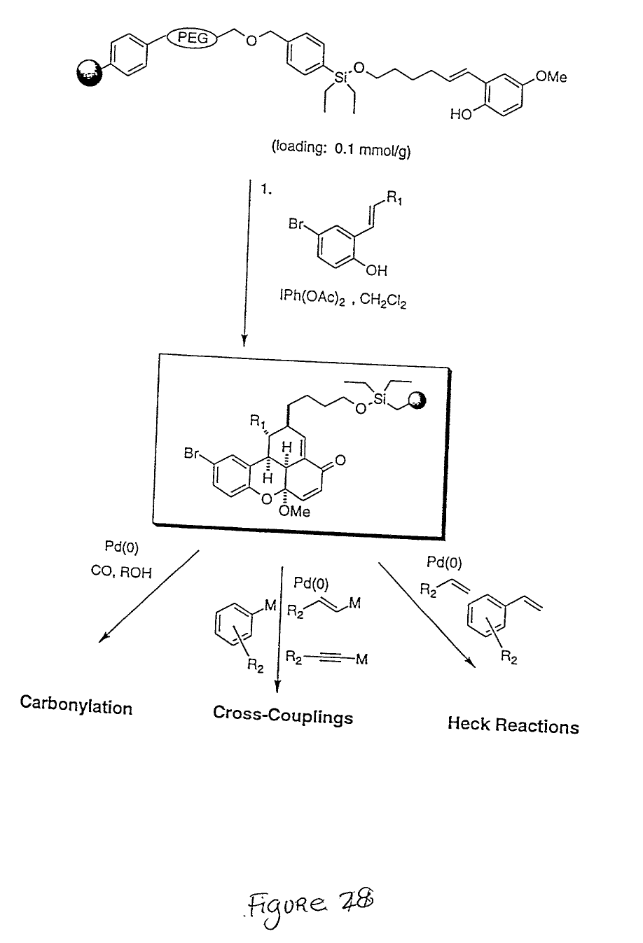

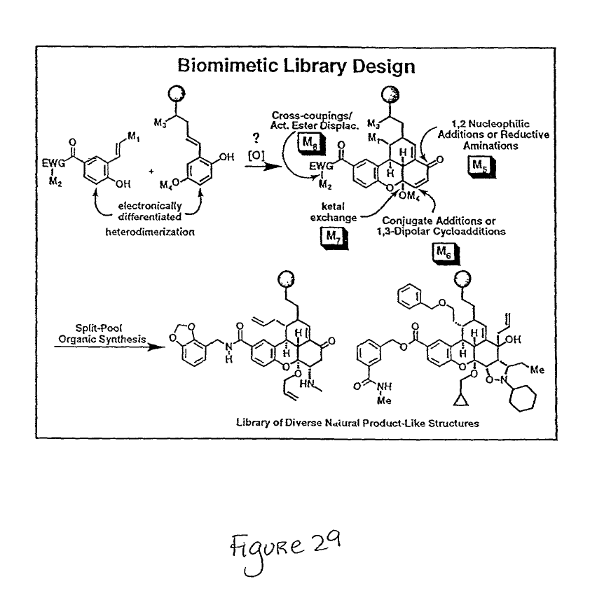

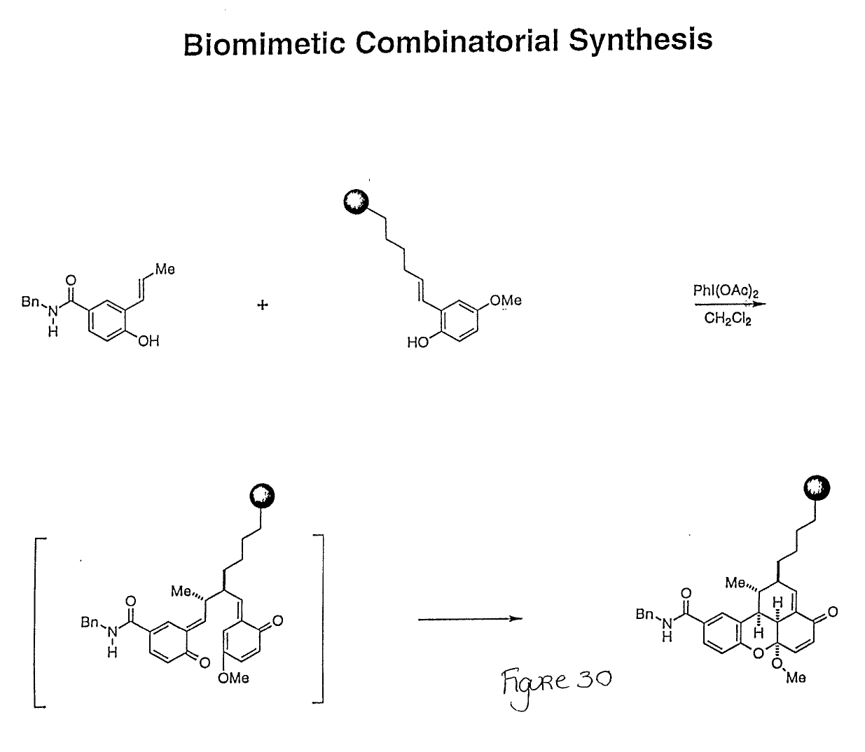

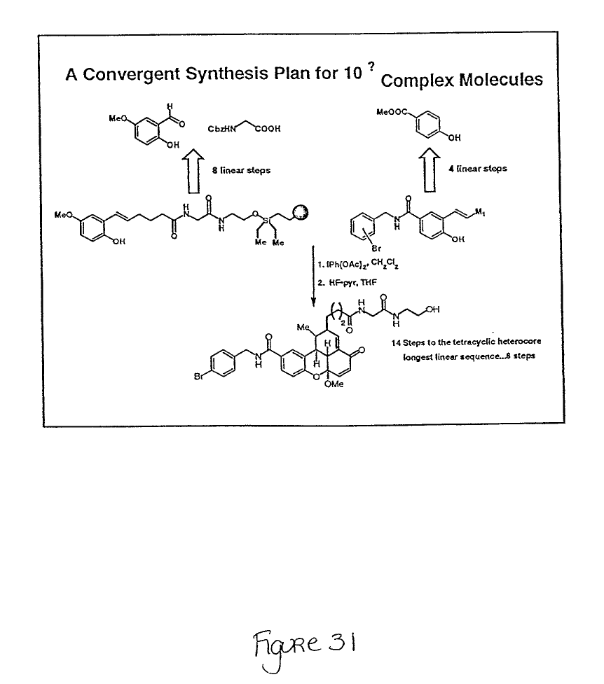

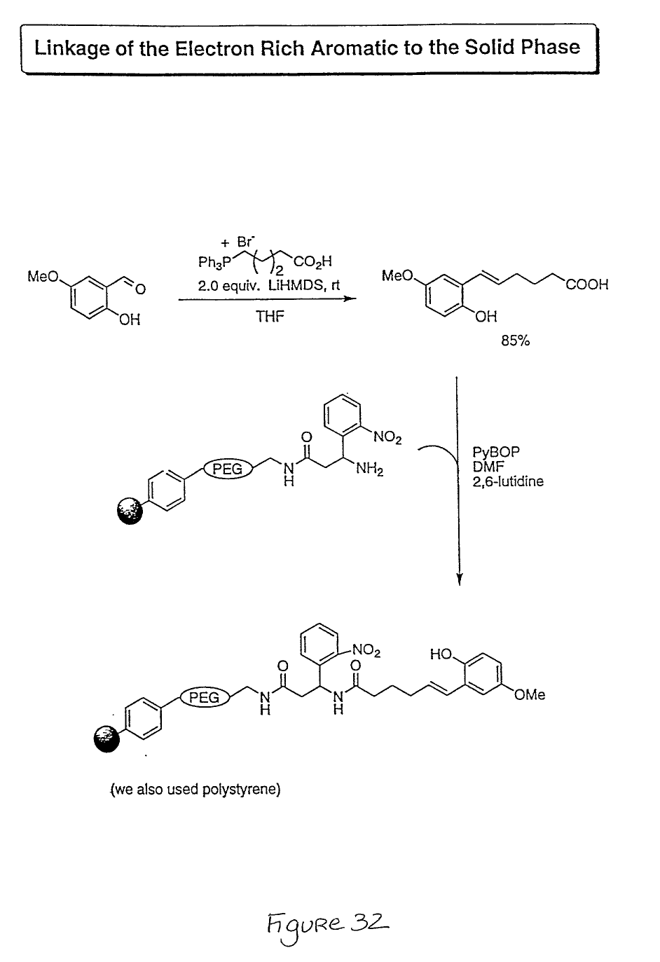

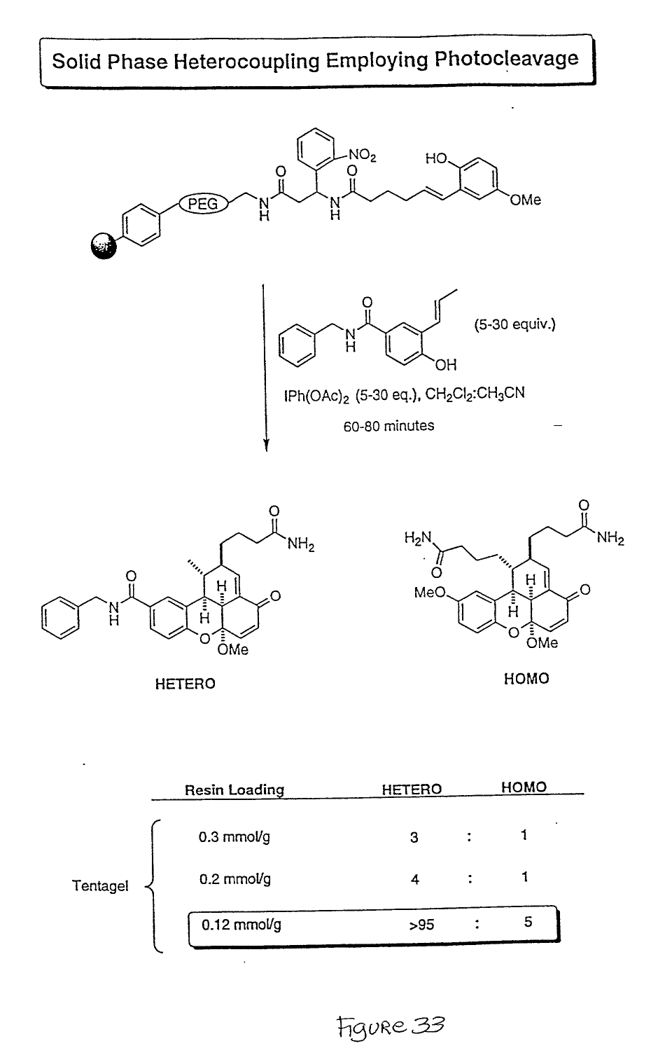

Furthermore, a specific application of this reaction to achieve the synthesis of the core structure of natural products CP-263,114 and CP-225,917 is depicted in FIG. 20 and described in Appendix B. One of ordinary skill in the art will realize that the size of the ring systems and the functionality present may be varied to yield alternative bicyclo ring systems. In one example, the facile synthesis of a taxane skeleton can be achieved using the inventive method. In this example, the 2-vinyl-2-carbomethoxy cycloalkanone is a hexanone system with a fused aromatic ring, as shown in FIG. 21. FIGS. 22, 23, and 24 additionally depict the use of the inventive method to apply to complex bridgehead olefin containing molecules to generate increasingly diverse and complex natural product-like compounds.

In another particularly preferred embodiment of the presently claimed invention, a method for the cascade synthesis of medium ring structures is provided, as shown in Equation 7, and more generally in FIG. 25, by using diethyl ether as a solvent.

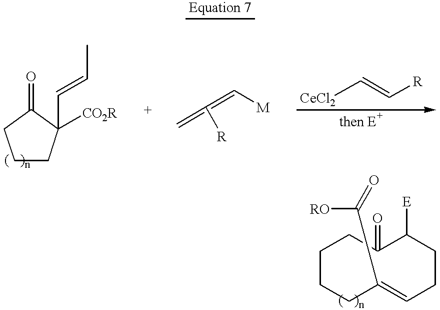

In particularly preferred embodiments, n is 0-3, and thus 9-12 membered rings can be generated, respectively.

In another particularly preferred embodiment , the ring systems depicted above can be reacted further, in a Michael-type transannular cyclization, to generate diastereoselective complex fused ring systems, as shown in FIG. 26. In preferred embodiments, 5,6; 6,6; 7,6; and 8,6 fused ring systems are generated, however, one of ordinary skill in the art will realize that any ring system may be generated with the limitation that the ring structure is stable.

For each of these compounds derived from cascade reactions, the functionalities emanating from the carbon skeleton of the structures are each independently selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocycl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, or any derivative incorporating phosphorous; and any of the functionalities taken together may also form a carbocycle or heterocycle having from 3 to 10 atoms in the ring. Furthermore, although a carbon-based skeleton is depicted, one of ordinary skill in the art will realize that skeletons incorporating sulfur, oxygen, or nitrogen are also possible using the present inventive method.

Reactions Functionalities in the Inventive Scaffold Structures

Once the inventive scaffolds have been synthesized as discussed above, diversification reactions may be employed at each of the different latent functionality sites present in the scaffold. One of ordinary skill in the art will appreciate that the reagents chosen for reaction at the latent functionality will only be limited by the reactivity of that reagent with that particular functionality.

In but one example of a particularly preferred embodiment, diversification reactions are employed on the heterodimerization product scaffold, as shown in FIG. 27, to generate the inventive libraries.

Specific reactions to which some or all of the heterodimerization product scaffold were subjected include reactions with nucleophiles at alkenyl moieties, including but not limited to hydroxyls, aminos, and thiols. Furthermore, reaction at a hydroxyl moiety with a vinyl aldehyde or vinylalkoxide and iron sulfate in a ring opening reaction, generates a ten member ring that can also be further functionalized if desired. Also, reaction with alkenes or vinyl aldehydes or vinylalkoxides and zinc chloride yields expanded ring structures and generates further sites for diversification at R₇ and R₈. Finally, cleavage of a the ring with an alkynyl reagent, yields a diversifiable alkenyl site. FIG. 28 also depicts some preferred reactions to be employed on the diversifiable biomimetic scaffolds, in particular carbonylations, transition metal mediated cross coupling reactions and Heck reactions. Additional reactions and resultant libraries are also shown in FIG. 29 in which 1,2 nucleophilic additions, conjugate additions and cross couplings are utilized, to name a few.

Furthermore, similar diversification reactions may be employed at appropriate functionalities on the homodimerization products, intramolecular dimerization products, and on the polycyclic, ring opened, and fused ring scaffold structures. Exemplary diversification reactions employed on these cascade scaffold structures include, but are not limited to Diels-Alder and hetero Diels-Alder reactions, conjugate additions, radical fragmentations, olefin metathesis, and palladium π-allyl substitutions.

Additionally, for each of the inventive compounds and libraries of compounds discussed, further reactions may be employed to attach biomolecules, polymers or solid support units to appropriate functionalities.

As one of ordinary skill in the art will realize, the above described reactions are merely exemplary of the types of reactions that may be performed on the inventive scaffolds. Other reactions may easily be substituted or added, with the limitation that the reactions utilized be compatible with the scaffold utilized. The full arsenal of synthetic chemistry is intended to be employed for the production of biomimetic compounds and libraries of compounds.

Combinatorial Synthesis of Biomimetic Libraries

According to the method of the presently claimed invention, the synthesis of libraries form the above-described scaffold structures can be performed in solution or on a solid support. One of ordinary skill in the art will realize that the choice of method will depend upon the specific number of compounds to be synthesized, the specific reaction chemistry, and the availability of instrumentation, such as robotic instrumentation for the preparation and analysis of the inventive libraries. The attachment of the scaffold structures to the solid support is particularly preferred because it enables the use of more rapid split and pool techniques to generate libraries containing as many or more than 1,000,000 members.

In one preferred embodiment, for the generation of a solution phase combinatorial library, a parallel synthesis technique is utilized, in which all of the products are assembled separately in their own reaction vessels. In a particularly preferred parallel synthesis procedure, a microtitre plate containing n rows and m columnns of tiny wells which are capable of holding a few milliliters of the solvent in which the reaction will occur, is utilized. It is possible to then use n variants of reactant A, and m variants of reactant B, to obtain n×m variants in n×m wells. One of ordinary skill in the art will realize that this particular procedure is most useful when smaller libraries are desired.

In another more particularly preferred embodiment of the presently claimed invention, a solid phase synthesis technique is utilized, in which the desired scaffold structures are attached to the solid phase directly or through a linking unit. Advantages of solid phase techniques include the ability to more easily conduct multi-step reactions and the ability to drive reactions to completion because excess reagents can be utilized and the unreacted reagent washed away. Perhaps one of the most significant advantages of solid phase synthesis is the ability to use a technique called “split and pool”, in addition to the parallel synthesis technique, developed by Furka. In this technique, a mixture of related compounds can be made in the same reaction vessel, thus substantially reducing the number of containers required for the synthesis of very large libraries, such as those containing more than one million library members. As an example, the solid support scaffolds can be divided into n vessels, where n represents the number of species of reagent A to be reacted with the scaffold structures. After reaction, the contents from n vessels are combined and then split into m vessels, where m represents the number of species of reagent B to be reacted with the scaffold structures. This procedure is repeated until the desired number of reagents is reacted with the scaffold structures to yield the inventive library.

The use of solid phase techniques in the presently claimed invention may also include the use of a specific encoding technique. As used in the presently claimed invention, in one aspect an encoding technique involves the use of a particular “identifying agent” attached to the solid support, which enables the determination of the structure of a specific library member without reference to its spatial coordinates. In another aspect, particularly if smaller libraries are generated in specific reaction wells, such as 96 well plates, or on plastic pins, the encoding information of these library members may also be identified by their spatial coordinates, and thus do not utilize an “identifying agent” attached to the solid support.

Examples of particularly preferred encoding techniques that can be utilized in the presently claimed invention include, but are not limited to graphical encoding techniques, including the “tea bag” method, chemical encoding methods, and spectrophotometric encoding methods. Graphical encoding techniques involve the coding of each synthesis platform to permit the generation of a relational database. Spectrophotometric encoding methods are useful for the presently claimed invention if no cleavage of the library member from the solid support is desired. An example of a preferred spectrophotometric encoding technique is the use of nuclear magnetic resonance spectroscopy. In a most preferred embodiment, chemical encoding methods are utilized. Decoding using this method can be performed on the solid support or cleaved from the solid support. One of ordinary skill in the art will realize that the particular encoding method to be used in the presently claimed invention must be selected based upon the number of library members desired and the reaction chemistry.

In one particularly preferred embodiment, the synthesis of over one million compounds having structural features reminiscent of natural products can be achieved using an encoded split and pool technique. FIG. 30 shows the general plan for one of the libraries of the inventive method. In this method, one of the phenols to be utilized in the inventive reaction is attached to the solid phase, using a means for attachment. Once the solid phase synthesis of the desired biomimetic tetracycle has been completed, diversification reactions can be employed to generate a library of biomimetic compounds. FIG. 31 also depicts a plan for the convergent synthesis of a large number of natural product-like compounds.

A solid support, for the purposes of this invention, is defined as an insoluble material to which compounds are attached during a synthesis sequence. The use of a solid support is advantageous for the synthesis of libraries because the isolation of support-bound reaction products can be accomplished simply by washing away reagents from the support-bound material and therefore the reaction can be driven to completion by the use of excess reagents. Additionally, the use of a solid support also enables the use of specific encoding techniques to “track” the identity of the inventive compounds in the library. A solid support can be any material which is an insoluble matrix and can have a rigid or semi-rigid surface. Exemplary solid supports include but are not limited to pellets, disks, capillaries, hollow fibers, needles, pins, solid fibers, cellulose beads, pore-glass beads, silica gels, polystyrene beads optionally cross-linked with divinylbenzene, grafted co-poly beads, poly-acyrlamide beads, latex beads, dimethylacrylamide beads optionally crosslinked with N-N′-bis-acryloylethylenediamine, and glass particles coated with a hydrophobic polymer. One of ordinary skill in the art will realize that the choice of a particular solid support is only limited by the compatibility of the support with the reaction chemistry being utilized. In one particularly preferred embodiment, a Tentagel amino resin, a composite of 1) a polystyrene bead crosslinked with a divinylbenzene and 2) PEG (polyethylene glycol), is employed for use in the presently claimed invention. Tentagel is a particularly useful solid support because it provides a versatile support for use in on-bead or off-bead assays, and it also undergoes excellent swelling in solvents ranging from toluene to water.

The compounds of the presently claimed invention may be attached directly to the solid support or may be attached to the solid support through a linking reagent. Direct attachment to the solid support may be useful if it is desired not to detach the library member from the solid support. For example, for direct on-bead analysis of biological activity or analysis of the compound structure, a stronger interaction between the library member and the solid support may be desirable. Alternatively, the use of a linking reagent may be useful if more facile cleavage of the inventive library members from the solid support is desired.

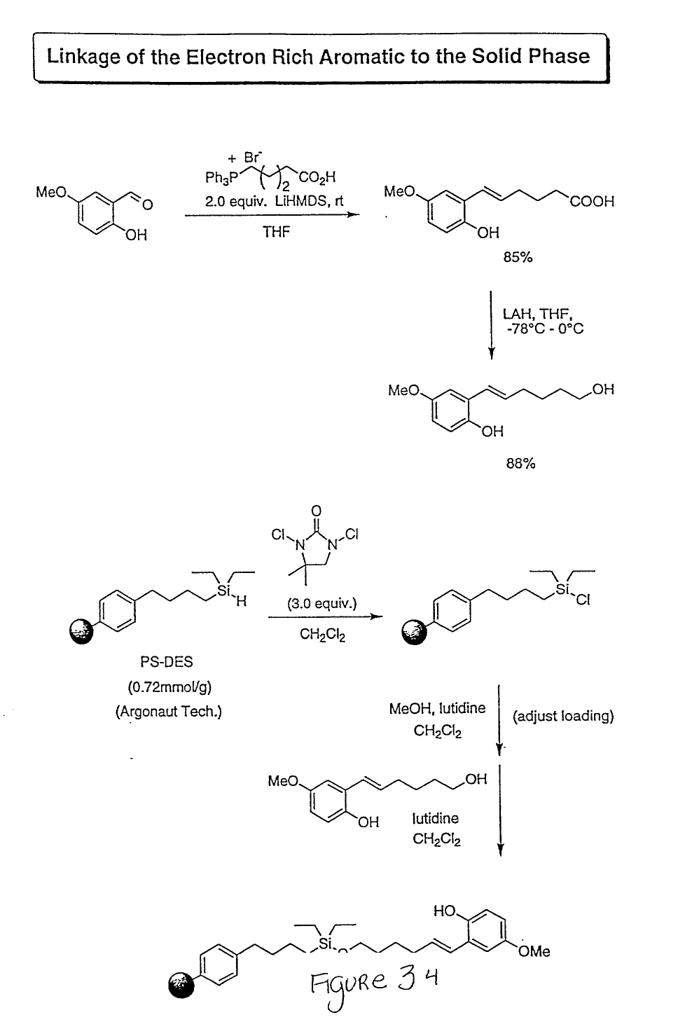

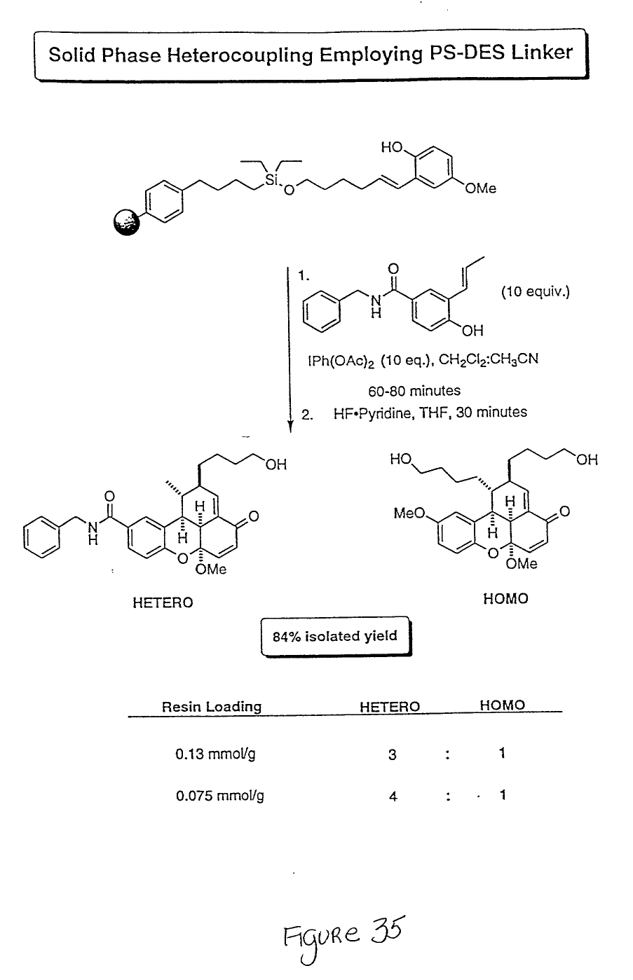

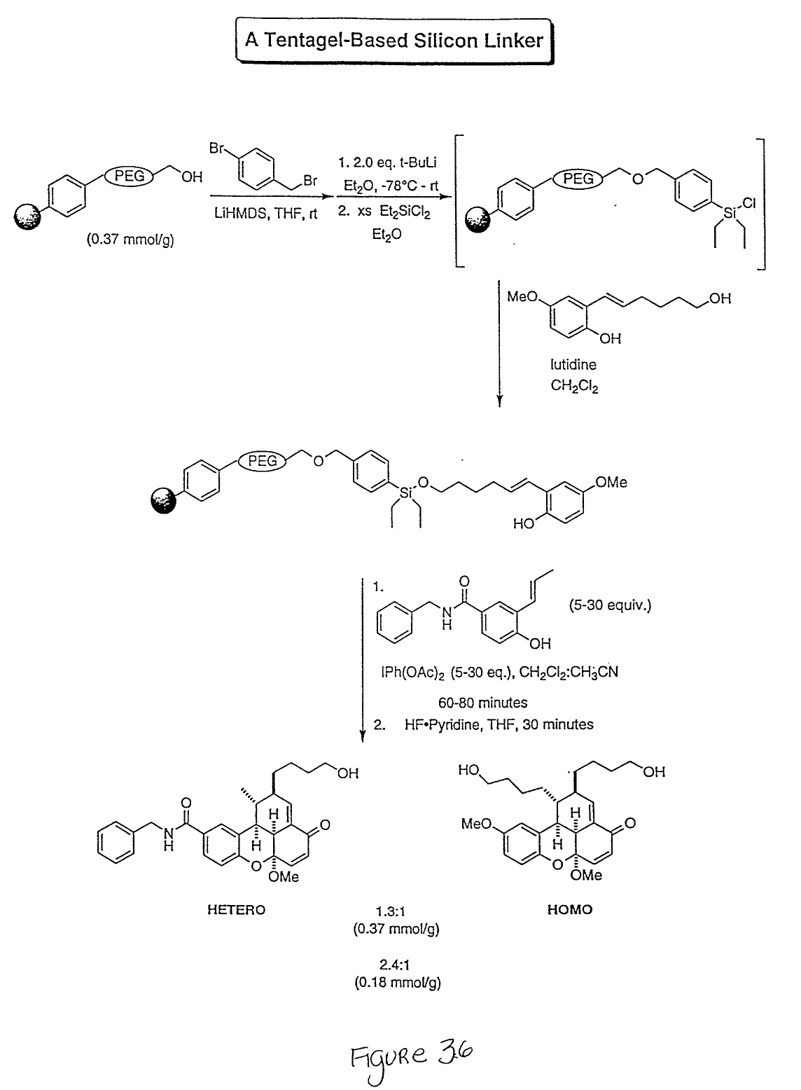

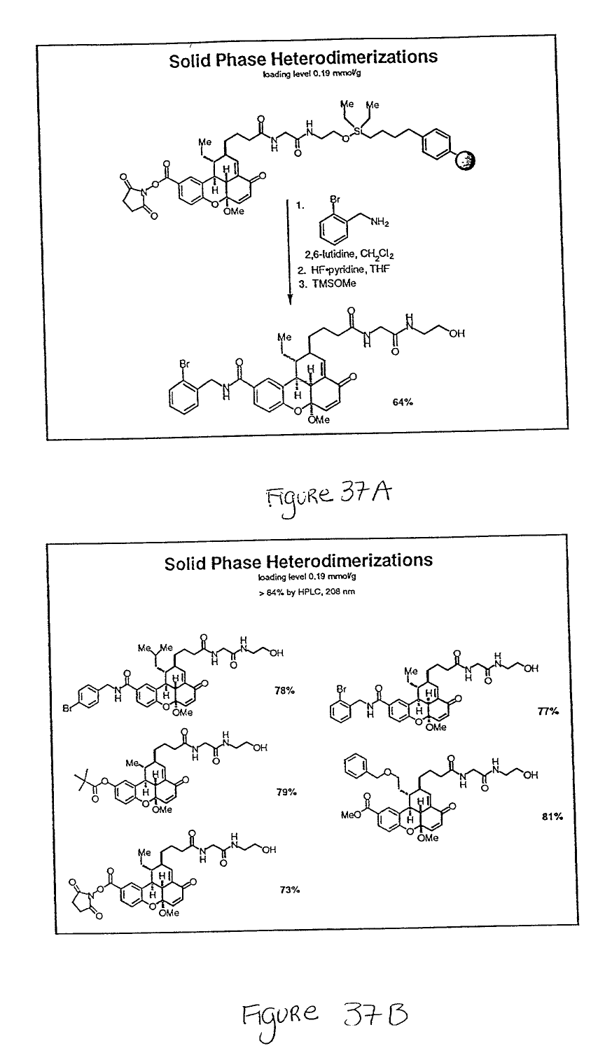

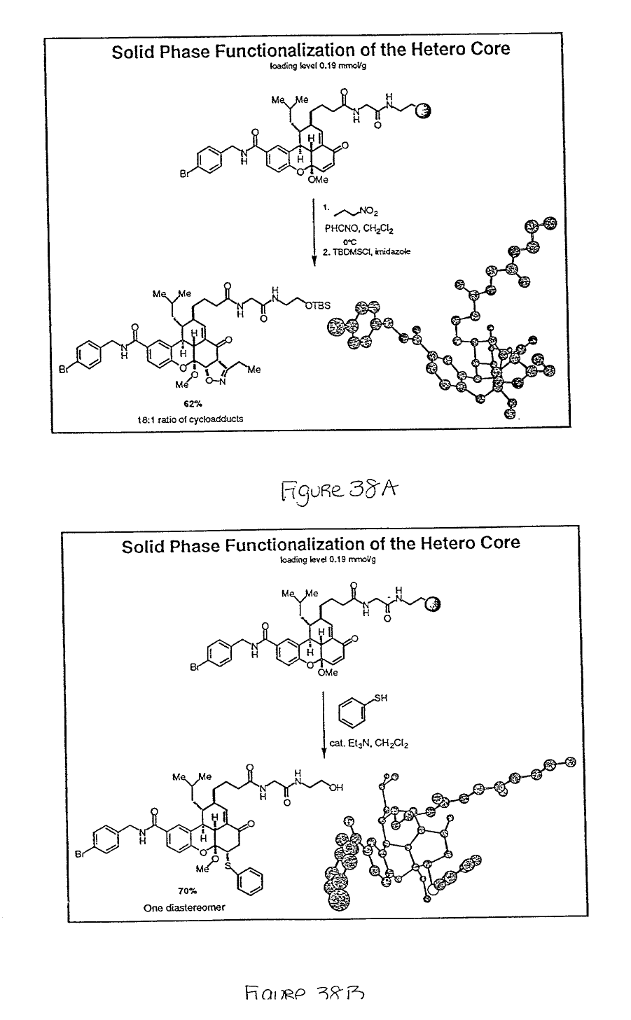

Any linking reagent used in the presently claimed invention may comprise a single linking molecule, or alternatively may comprise a linking molecule and one or more spacer molecules. A spacer molecule is particularly useful when the particular reaction conditions require that the linking molecule be separated from the library member, or if additional distance between the solid support/linking unit and the library member is desired. In one preferred embodiment, photocleavable linkers are employed to attach the solid phase resin to the desired phenol as shown in FIGS. 32 and 33. Photocleavable linkers are particularly advantageous for the presently claimed invention because of the ability to use these linkers in in vivo screening strategies. Once the template is released from the solid support via photocleavage, the complex small molecule is able to enter the cell. Other preferred linkers include silicon linkers as depicted in FIGS. 34 and 35.

A particularly preferred linker for use in the presently claimed invention is a tentagel-based silicon linker, an inventive linker developed specifically for the method of the presently claimed invention. The synthesis of this linker and its use in the presently claimed invention is depicted in FIG. 36. This linker represents the first silicon based linker utilized with Tentagel (polystyrene and polyethylene glycol). As a result, the advantages of utilizing a silicon linker can be coupled with the advantageous characteristics of Tentagel such as greater swelling of the beads and greater diffusibility of reagents. This linker is preferably synthesized using hydoroxymethyl Tentagel, para-bromobenzyl bromide and a silicon reagent such as diethyldichlorosilane (Et₂SiCl₂). One of ordinary skill in the art will realize that other silicon moieties such as diisopropyl, dimethyl, diphenyl and other substituted diaryl, dimesityl, dicyclohexyl, and di-tert-butylsilyl dichlorides may be utilized, although the inventive method for this linker is not limited to these alternative reagents. Additionally, the presently claimed method is not limited to the linkers described above; rather, other linkers that are compatible with the reaction chemistry being utilized an be employed for use in the presently claimed invention.

Additionally, as discussed above in the context of control of heterodimerization, in preferred embodiments, the use of amides along the chain linking the electron rich phenol to the solid phase is utilized. In particularly preferred embodiments, as shown in FIG. 10, a linking system comprising glycine and 2-aminoethanol is utilized.

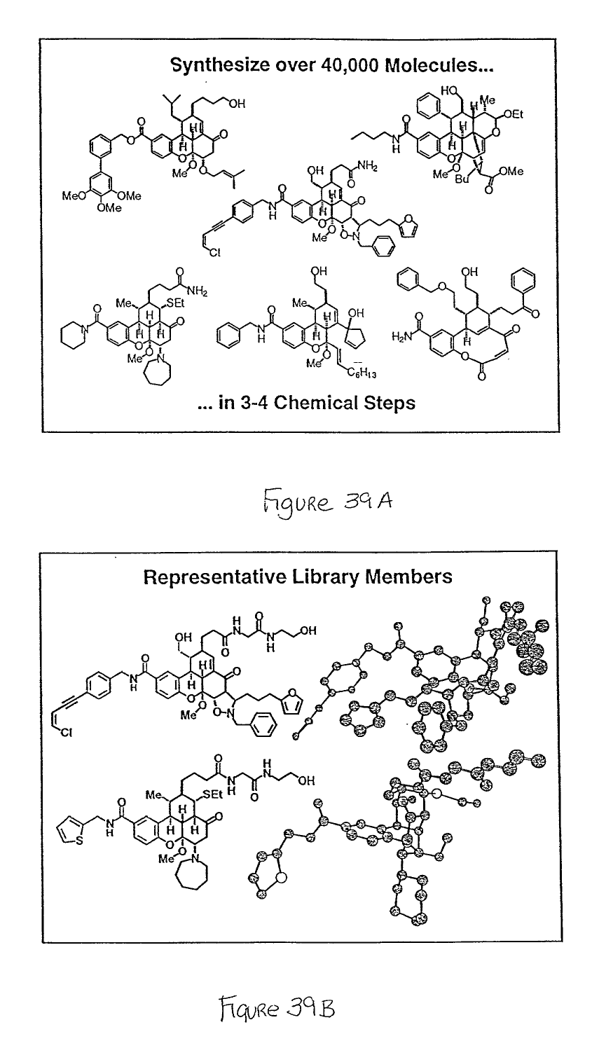

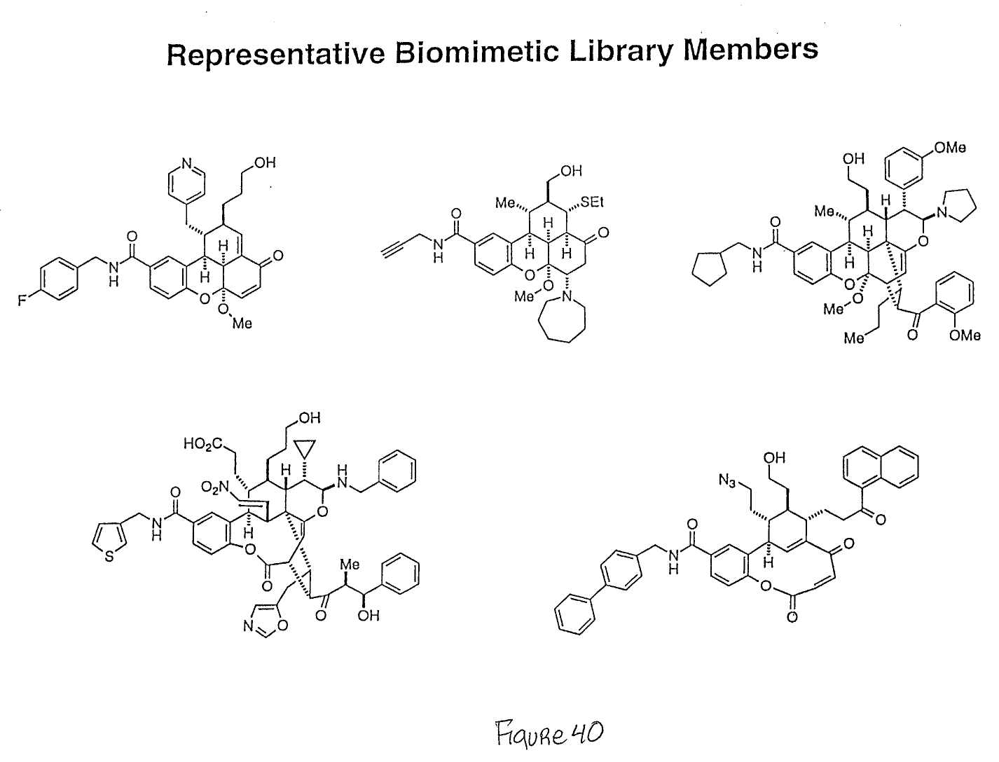

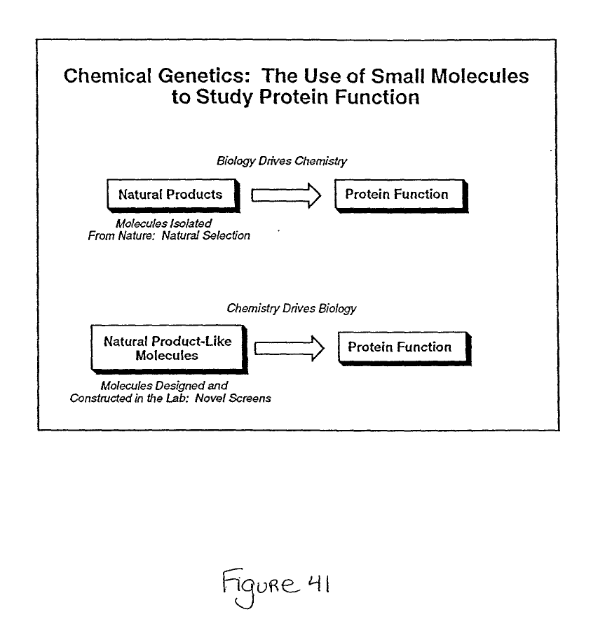

One of ordinary skill in the art will realize that the reagent selected for attachment to the solid phase will be selected for its ability, in the case of heterodimerization, to favor a reaction between the attached reagent and the non-attached phenol. In a particularly preferred embodiment the “hot” phenol, or the electron rich phenol, is selected for attachment to the solid phase and is subsequently reacted with the electron deficient phenol to yield the heterodimerization scaffold product. After the synthesis of this scaffold structure, functionalization of the sites can be performed in a combinatorial fashion, using a split and pool method in a preferred embodiment, where the scaffold structures are split into n batches, and reacted with any combination of n reagents or “blanks” at a particular functionality. It is important to note that “blanks”, a term used in the context of the present invention to represent the purposeful omission of reaction with a any particular reagent, are also tools utilized for the generation of additional diversity. After this reaction sequence, the beads can be tagged and pooled, and then split again into n batches and reacted with n reagents or “blanks”. This sequence can be repeated for the inventive method until the desired library is achieved. FIGS. 37A and 37B depict exemplary solid phase heterodimerizations to achieve desired functionalizable core structures. FIGS. 38A and 38B show the solid phase functionalization of specific core structues. Furthermore, FIGS. 39A, 39B, and 40 depict several representative biomimetic library members. One of ordinary skill in the art will realize that the presently claimed invention is not limited to the split and pool method and that other combinatorial methods may be employed. Furthermore, it will be appreciated that the reactions depicted in the specification and the figures are merely representative of the inventive methods and libraries and the present invention encompasses the full scope of reactivity and structures possible.

Uses

The methods, compounds and libraries of the present invention can be utilized in various disciplines. Many of the natural products upon which these compounds are based have important biological and therapeutic activities, including, antiviral (pretazzine) and inhibitors of ACE (galanthamine), to name a few. The inventive natural product-like compounds and libraries of compounds are thus expected to have important biological and therapeutic activities. Any available method may be employed to screen the libraries produced according to the present invention to identify those with desirable characteristics for a selected application. As mentioned previously, one of the goals of the emerging field of chemical genetics is to utilize complex small molecules to alter, i.e. inhibit or initiate, the action of proteins as shown in FIG. 41. In the method of the present invention, one or more compounds of the presently claimed invention may be subjected to a biological target having a detectable biochemical activity. Such biological targets can be in the form of enzymes, receptors, subunits involved in the formation of multimeric complexes, and having such biochemical activities such as substrate conversion (catalysis of chemical reactions) or merely the ability to bind to another molecule. The biological target can be provided in the form of a purified or semi-purified composition, a cell lysate, a whole cell or tissue, or even a whole organism. The level of biochemical activity is detected in the presence of the compound, and a statistically significant change in the biochemical activity, relative to the level of biochemical activity in the absence of the compound, identifies the compound as a modulator, e.g. inhibitor or potentiator of the biological activity of the target protein.

In one particularly preferred embodiment, a miniaturized assay system is utilized. The ability of the preferred procedure utilized for the library synthesis to controllably release compounds from the individual 90μ diameter beads into nanodroplet containing engineered cells enables the use of these miniaturized cell-based assays to detect specific characteristics of library members. In a particularly preferred embodiment of the invention, the compounds in the encoded combinatorial library are attached to beads through a photocleavable linker. Each bead is labeled with a tag that identifies the bound compound. Additionally, the concentration of the test compound released in the droplet can be controlled by controlling the time of exposure to UV radiation. Additionally, the amount of compound released in any particular experiment, of course, will depend on the efficiency of bead loading and the extent of bead functionalization. Those of ordinary skill in the art will readily appreciate that any of a wide variety of read-out assays can be employed with the assay system described above. Any assay whose result may be observed in the context of a discrete liquid droplet is appropriate for use with the present invention. Preferred read-out assays for use in accordance with the present invention analyze chemical or biological activities of test compounds. Read-out assays can be designed to test in vitro or in vivo activities.

Furthermore, the inventive compounds produced by the presently claimed invention can be provided as a kit comprising a specific library of compounds, and a reagent for determining one or more biological activities of the biomimetic library, such as a miniaturized assay system consisting of a specific assay to detect inhibition of promotion of a particular cellular function.

Alternatively, once a specific desired activity has been associated with a particular compound of the inventive library, the compounds of the presently claimed invention may be utilized as a therapeutic agent for a particular medical condition. A therapeutic agent for use in the present invention may include any pharmacologically active substances that produce a local or systemic effect in animals, preferably mammals, or humans. The term thus means any substance intended for use in the diagnosis, cure, mitigation, treatment or prevention of disease or in the enhancement of desirable physical or mental development and conditions in an animal or human. The therapeutic agent may be administered orally, topically or via injection by itself, or additionally may be provided as a pharmaceutical composition comprising the therapeutic agent and a biologically acceptable carrier. The inventive compositions can be, but are not limited to aqueous solutions, emulsions, creams, ointments, suspensions, gels, liposomal suspensions, and salts. Particularly preferred biologically acceptable carriers include but are not limited to water, saline, pills, capsules, tablets, syrups, Ringer's solution, dextrose solution and solutions of ethanol, glucose, sucrose, dextran, mannose, mannitol, sorbitol, polyethylene glycol (PEG), phosphate, acetate, gelatin, collagen, Carbopol, and vegetable oils tablets. It is also possible to include suitable preservatives, stabilizers, antioxidants, antimicrobials, and buffering agents, for example including but not limited to BHA, BHT, citric acid, ascorbic acid, and tetracycline. The therapeutic agents of the presently claimed invention may also be incorporated or encapsulated in a suitable polymer matrix or membrane, thus providing a sustained-release delivery device suitable for implantation near the site to be treated locally.

As one of ordinary skill in the art will realize, the amount of the therapeutic agent required to treat any particular disorder will of course vary depending upon the nature and severity of the disorder, the age and condition of the subject, and other factors readily determined by one of ordinary skill in the art.

Additionally, although the inventive libraries are particularly suited for use in biological and medical applications, they may also be useful in the fields of catalysis, as novel ligands for catalyst design, and materials science. The diversifiability of these biomimetic compounds may enable the attachment of novel materials, in addition to biomolecules.

Equivalents

Those skilled in the art will recognize, or be able to ascertain using no more than routine experimentation, numerous equivalents to the inventive libraries and methods of use thereof described herein. Such equivalents are considered to be within the scope of this invention and are covered by the following claims. Additionally, examples of particularly preferred embodiments are presented in the examples below and are intended to more particularly describe the present invention, but are not intended to limit the scope of the presently claimed invention.

Examples

1. Intermolecular oxidative phenolic couplings: see Appendix A

2. Intramolecular oxidative phenolic couplings:

General Procedures: All reactions were performed in oven-dried glassware under a positive pressure of argon except in the preparation of 8 (Figure X). Flash chromatography was performed as described by Still et al. (Still, W. C.; Kahn, M.; Mitra, A. J. Org. Chem. 1978, 43, 2923).

Materials: Tetrahydrofuran and diethyl ether were distilled under nitrogen from sodium-benzophenone ketyl. Dichloromethane, diisopropylethylamine, and 2,6-lutidine were distilled under nitrogen from calcium hydride.

Instrumentation: Infared spectra were recorded on a Perkin-Elmer 1600 series FT-IR spectrometer. ₁H and ₁₃C NMR were recorded on either a Bruker AM500 (500 MHz/125 MHz), Bruker AM400 (400 MHz/100 MHz). Chemical shifts for proton and carbon resonances are reported in ppm (δ) relative to chloroform (7.26). All structures described below correspond to numbered structures in FIGS. 18A and 18B.

Preparation of 2. To 1 (25 g, 107.9 mmol) dissolved in 500 ml TH and 250 ml dichloromethane was added diisopropyl ethylamine (56.5 ml, 322.5 mmol) dropwise with stirring at room temperature. The solution was cooled to 0° C. After 20 minutes allylchloroformate (11.5 ml, 107.9 mmol) was added dropwise. After 1.5 hours the reaction was quenched with 100 ml saturated aqueous ammonium chloride. The solution was concentrated in vacuo, extracted into ether (4×300 ml), washed with saturated aqueous sodium chloride, and dried over anhydrous magnesium sulfate. Concentration in vacuo followed by column chromatography afforded 2 (30 g, 96%).

Preparation of 3. To 2 (30 g, 107.4 mmol) dissolved in 170 ml dichloromethane was added potassium carbonate (29.6 g, 214 mmol) followed by the dropwise addition of allylbromide (10.2 ml, 116. 3 mmol). After 9 hours the reaction was quenched with 500 ml water, extracted into ether (5×300 ml), washed with water, and dried over anhydrous magnesium sulfate. Concentration in vacuo followed by column chromatography afforded 3 (31.7 g, 92%).

Preparation of 4. To 3 (1.165 g, 3.65 mmol) dissolved in 10 ml THF and stirred at room temperature was added anhydrous lithium chloride (0.31 g, 7.3 mmol), sodium borohydride (0.28 g, 7.3 mmol), and ethanol (10.2 ml, 176 mmol). After 19 hours, a 10% aqueous citric acid solution was added to achieve pH 3 (5 ml). Water (20 ml) was added and the solution was extracted into ethylacetate (3×75 ml), and dried over anhydrous magnesium sulfate. Concentration in vacuo followed by column chromatography afforded 4 (0.85 g, 80%).

Preparation of 5. To 4 (4.54 g, 15.6 mmol) dissolved in 150 ml dichloromethane and cooled to 0° C. was added diisopropylethylamine (8.1 ml, 46.7 mmol) and triisopropylsilyltriflate (4.4 ml, 15.6 mmol) dropwise with stirring. After 2 hours, additional triisopropylsilyltriflate was added (1 ml). After 30 minutes, the reaction was quenched with 50 ml saturated aqueous ammonium chloride and extracted into dichloromethane (3×100 ml). Concentration in vacuo followed by column chromatography afforded 5 (6 g, 87%).

Preparation of 6. To 5 (0.78 g, 1.7 mmol) dissolved in 17.5 ml THF was added tetrakis(triphenylphosphine)palladium (0) (0.2 g, 0.17 mmol) and morpholine (1.5 ml, 17.1 mmol). The solution was stirred under argon at 47° C. After 12 hours, the solution was cooled to room temperature. Concentration in vacuo followed by column chromatography afforded 6 (0.542 g. 96%).

Preparation of 8. (J. R. Cannon, T. M. Cresp, B. W. Metcalf, M. V. Sargent, and G. Vinciguerra, J. Chem. Soc. 1971, 3495.). To 7 (35 g, 227 mmol) dissolved in water (454 ml) was added bromine (11.7 ml) dissolved in water (1250 ml) dropwise with stirring at room temperature. The solution was refluxed for 7 days. Concentration in vacuo followed by recrystallization from water afforded 8.

Preparation of 9. To 8 (0.5 g, 2.1 mmol) dissolved in anhydrous DMF was added potassium carbonate (1.48 g, 10.7 mmol). Allylbromide (0.65 ml, 7.51 mmol) was subsequently added dropwise at room temperature. After 12 hours, the reaction was quenched with 30 ml water, extracted into ether (4×25 ml), washed with water, and dried over magnesium sulfate. Concentration in vacuo (82% crude yield) followed by column chromatography afforded 9.

Preparation of 10. To 9 (4.86 g, 13.8 mmol) dissolved in 110 ml THF and cooled to 0 C. was added lithium aluminum hydride (1M solution in THF, 27.5 mmol) dropwise. After one hour, saturated aqueous Rochelle's salt was added dropwise until bubbling ceased. Ether was then added and the suspension was filtered through celite and concentrated in vacuo. The aqueous suspension was diluted with 20 ml of water, extracted into ethylacetate (4×60 ml), washed with saturated aqueous sodium chloride and dried over magnesium sulfate. Concentration in vacuo (crude yield 81%) followed by column chromatography afforded 10.

Preparation of 11. To a suspension of pyridinium chlorochromate (4.59 g, 21.3 mmol), celite (4.59 g), and sodium acetate (0.44 g, 5.3 mmol) in dichloromethane (62 ml) at 0 C. was added 10 (3.19 g, 10.6 mmol) dissolved in 31 ml dichloromethane via canula. After 3 hours, the suspension was decanted into 300 ml ether and stirred at room temperature. After 2 hours, the suspension was filtered through celite. Concentration in vacuo followed by column chromatography afforded 11 (2.51 g, 79%).

Preparation of 12. To 6 (2.43 g, 7.5 mmol) was added 11 (2.24 g, 7.5 mmol) dissolved in 110 ml anhydrous methanol via canula. The solution was cooled to 0° C. Wet acetic acid (5 ml) was added dropwise with stirring. After 30 minutes, sodium cyanoborohydride was added (250 mg, 4.0 mmol). A second aliquot of sodium cyanoborohydride (226.5 mg, 3.6 mmol) was added after 30 minutes. The reaction was allowed to slowly warm to room temperature from 0° C. After 12 hours, the reaction was quenched with 100 ml of saturated aqueous sodium chloride and 150 ml of 10% sodium hydroxide in saturated aqueous sodium chloride, extracted into 5% hexanes in ethylacetate (4×200 ml), washed with 15 ml saturated aqueous sodium chloride, and dried over magnesium sulfate. Concentration in vacuo followed by column chromatography afforded 12.

Preparation of 13a. To 12 (1 g, 1.75 mmol) dissolved in 10 ml THF was added 2,6 lutidine dropwise with stirring at room temperature followed by allylchloroformate (176 ul, 1.66 mmol). After five minutes, 30 ml of THF was added. After 12 hours, the reaction was quenched with 10 ml saturated aqueous ammonium chloride, concentrated in vacuo, extracted into dichloromethane (4×200 ml), and dried over magnesium sulfate. Concentration in vacuo followed by column chromatography yielded 13a (1.24 g, quantitative).

Preparation of 13b. To 12 (0.31 g, 0.5 mmol) dissolved in 5 ml dichloromethane was added 2,6 lutidine dropwise with stirring at room temperature followed by cooling to 0° C. FMOC-chloride (0.14 g, 0.5 mmol) was added. The reaction was allowed to warm to room temperature. After 19 hours, the reaction was quenched with 30 ml saturated aqueous ammonium chloride, extracted into dichloromethane (3×50 ml), washed with saturated aqueous sodium chloride, and dried over magnesium sulfate. Concentration in vacuo followed by column chromatography afforded 13b.

Preparation of 14a (procedure is identical for 14b). To 13a (1.25 g, 1.8 mmol) dissolved in 20 ml of 2,2,2-trifluoroethanol was added propylene oxide (10 ml, 181 mmol). The solution was cooled to −40° C. and phenyliodine (III)bis(trifluoroacetate) (0.86 g, 2 mmol) dissolved in 10 ml of 2, 2, 2-trifluoroethanol was added dropwise with stirring. After 15 minutes, the solution was allowed to slowly warm to 0° C. over 30 minutes. Concentration in vacuo followed by column chromatography afforded 14a (0.8 g, 64%).

Preparation of 15. To 14a (643.1 mg, 0.9 mmol) dissolved in 15 ml THF was added morpholine (0.8 ml, 9.4 mmol) and tetrakis(triphenylphosphine) palladium (0) (100 mg, 0.09 mmol) at room temperature. After 1.5 hours, the solution was concentrated in vacuo. Column chromatography afforded 15 (508 mg, quantitative).

Preparation of 16. To 14b (12.5 mg, 0.015 mmol) dissolved in 1 ml dichloromethane was added piperidine (200 ul, 2 mmol) dropwise with stirring at room temperature. After 30 minutes, the solution was concentrated in vacuo affording 16.

3. Cascade reactions: see Appendix B.

### Experimentals for Biomimetic Combinatorial Synthesis Project

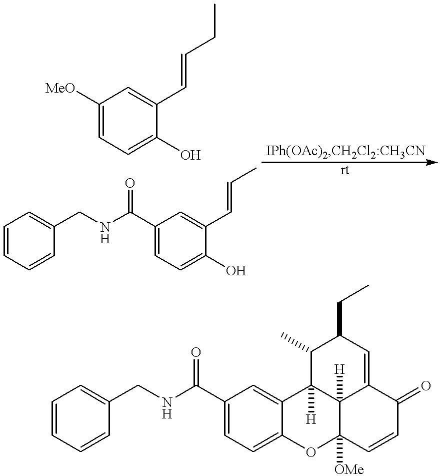

Heterodimerization in Solution

To an oven-dried flask, equipped with stir bar and septum and cooled/purged under a stream of Ar (g), was charged with 3-[(E)-1-propenyl]-4-hydroxy-N-benzylbenzamide (25 mg, 0.09 mmol) and 1-hydroxy-2-[(E)-1-butenyl]-4-methoxybenzene (100 mg, 0.56 mmol, 6.0 equiv.). Distilled CH₂Cl₂ (4 mL) was added via syringe followed by 3 drops of dry CH₃CN. Then, the septum was quickly opened, and iodobenzene diacetate (181 mg, 0.56 mmol, 6.0 equiv.) added in 1 portion. The reaction rapidly changed from water-white to yellow to orange, and finally deep red within five minutes of addition. By TLC, all starting amide was consumed within ten minutes. The reaction was poured into a separatory funnel, extracted into EtOAc and washed with saturated NaHCO₃, water, and brine. Concentration in vacuuo and preparative TLC afforded 25 mg (60%) of a single diastereomer as an off-yellow foam. TLC[hex:EtOAc, 50:50] R_{f}=0.26; ¹H NMR (500 MHz, CDCl₃): δ 7.92 (s, 1H), 7.45 (dd, J=1.5, Hz, 8.2 Hz,1H), 7.26 (m, 5H), 7.05 (m, 1H), 6.78 (d, J=8.2 Hz, 1H), 6.45 (t, J=5.6 Hz, 1H), 6.29 (d, J=10 Hz, 1H), 4.56 (qd, 6 Hz, 14.5 Hz, 2H), 3.3 (m, 1H), 3.26 (s, 3H), 3.15 (m, 1H), 2.75 (m, 1H), 1.85 (m, 1H), 1.1 (d, J=7 Hz, 3H), 0.9-0.6 (m, 4H), 0.8 (t, 3H); MS-FAB: 466 (M+23(Na)), 444 (M+1).

[chemical structure]

Heterodimerization on Solid Phase-Photolinker

To a 20 mL Biorad tube was placed the photolinker-based resin (1 g, 0.12 mmol, 0.12 mmol/g) and 3-[(E)-1-propenyl]-4-hydroxy-N-benzylbenzamide (160.2 mg, 0.6 mmol, 5.0 equiv.). Dry CH₂Cl₂ was added to swell the resin, followed by the minimum amount of CH₃CN to dissolve the amide. The Biorad tube was then placed on an orbital stirrer and allowed to stir for 30 minutes to afford good mixing. Then, the cap was quickly removed and iodobenzene diacetate (193 mg, 0.6 mmol, 5.0 equiv.) added in one portion. The cap was replaced, and the Biorad tube returned to the orbital stirrer and allowed to stir at rt for 2 hours. During this time, the solution/resin changed from pale yellow to deep red. After this time, the resin was washed (×8) with THF, CH₂Cl₂, MeOH, H₂O, and hexane followed by drying under vacuum. The washed/dried resin was then transferred to an epindorff tube, diluted with dry CH₃CN, and photolyzed at 350 nm for 45 minutes while being aggitated with a vortex stirrer. The contents of the epindorf tube were filtered and washed with THF into a 25 mL round-bottom flask and concentrated in vacuuo to afford a single diastereomer as yellow foam along with a trace of the product of homodimerization (>95:5). TLC[hex:EtOAc, 25:75] R_{f}=0.11; ¹H NMR (500 MHz, CDCl₃): δ 8.00 (s, 1H), 7.43 (dd, J=1.6 Hz, 8.4 Hz, 1H), 7.35 (m, 5H), 6.99 (m, 1H), 6.84 (d, J=8.4 Hz, 1H), 6.62 (t, J=5.8 Hz, 1H), 6.52 (bs, 1H), 6.34 (d, J=10.3 Hz, 1H), 5.3 (bs, 1H), 4.62 (qd, J=6 Hz, 14.7 Hz), 3.37 (m, 1H), 3.33 (s, 3H), 3.2 (dt, J=2.7 Hz, 4.6 Hz, 1H), 2.8 (m, 1H), 2.29 (m, 1H), 2.03 (m, 2H), 1.85 (m, 2H), 1.7 (m, 1H), 1.6 (m, 1H), 1.15 (d, J=7.2 Hz, 3H), 0.89 (m, 1H), 0.36 (M, 1H); MS-FAB: 523 (M+23 (Na)), 501 (M+1).

[chemical structure]

Heterodimerization on Solid Phase-Silicon Linker

To a 10 mL Biorad tube was placed the silicon linker-based resin (350 mg, 0.026 mmol, 0.075 mmol/g) and 3-[(E)-1-propenyl]-4-hydroxy-N-benzylbenzamide (104 mg, 0.39 mmol, 15.0 equiv.). Dry CH₂Cl₂ was added to swell the resin, followed by the minimnum amount of CH₃CN to dissolve the amide. The Biorad tube was then placed on an orbital stirrer and allowed to stir for 30 minutes to afford good mixing. Then, the cap was quickly removed, and iodobenzene diacetate (126 mg, 0.39 mmol, 15.0 equiv.) added in one portion. The cap was replaced, and the Biorad tube returned to the orbital stirrer and allowed to stir at rt for 2 hours. During this time, the solution/resin changed from pale yellow to deep red. After this time, the resin was washed (×8) with THF, CH₂Cl₂, MeOH, H₂O, and hexane followed by drying under vacuum. The washed/dried resin was then transferred to an epindorff tube, diluted with dry THF, and a few drops of HF•pyridine. The epindorff tube was placed on the orbital stirrer and allowed to proceed at rt for 45 minutes. The contents of the epindorff tube were filtered and washed with THF into a 25 mL round-bottomed flask and concentrated in vacuuo to afford a single diastereomer as an off-white foam, along with the product of homodimerization (4:1/hetero:homo). TLC[hex:EtOAc, 25:75] R_{f}=0.18; ¹H NMR (500 MHz, CDCl₃): δ 7.96 (s, 1H), 7.46 (dd, J=1.7 Hz, 8.5 Hz, 1H), 7.35 (m, 5H), 7.09 (m, 1H), 6.84 (d, J=8.5 Hz, 1H), 6.62 (t, J=5.8 Hz, 1H), 6.34 (d, J=10.3 Hz, 1H), 4.62 (qd, J=6 Hz, 14.7 Hz), 3.4 (m, 2H), 3.37 (m, 1H), 3.30 (s, 3H), 3.2 (dt, J=2.7 Hz, 4.6 Hz, 1H), 2.8 (m, 1H), 2.03 (m, 2H), 1.85 (m, 2H), 1.7 (m, 1H), 1.6 (m, 1H), 1.5 (m, 2H), 1.15 (d, J=7.0 Hz, 3H), 0.9 (m, 1H), 0.40 (m, 1H); MS-FAB: 498 (M+23 (Na)), 476 (M+1).

[chemical structure]

Preparation of a Tentagel-Based Silicon Linker

To a 25 mL Solid Phase Reaction Vessel (SPRV) was placed hydroxymethyl Tentagel (2 g, 0.74 mmol, 0.37 mmol/g) and purged with Ar (g). Dry THF ( 15 mL) was added, followed by the dropwise addition of a 1.0 M THF solution of LiN(TMS)₂ (0.8 mL, 0.8 mmol). In another dry flask, MeI (32.2 μL, 0.518 mmol) [Added to adjust the loading level to 0.11 mmol/g] and nara-bromobenzyl bromide (55 mg, 0.222 mmol) were dissolved in 5 mL of dry THF, and transferred via cannula to the lithium alkoxide resin. The SPRV was placed on an orbital stirrer and allowed to go overnight at rt. After this time, the resin was washed (×8) with THF, CH₂Cl₂, MeOH, H₂O, and hexane followed by drying under high vacuum. The washed/dried resin was then transferred to a Schlenk flask, purged with Ar(g), and swollen with freshly distilled Et₂O. The Schlenk was cooled to −78° C., whereupon t-BuLi (142 μL, 0.242 mmol, 1.2 equiv.) was added dropwise, and allowed to slowly warm to rt to complete the transmetallation. After 45 minutes at rt, excess Et₂SiCl₂ (250 μL, 1.65 mmol, 7.5 equiv.) was added via syringe. The Schlenk was occasionally aggitated over 2 hours at rt, and then the excess Et₂SiCl₂ was removed by filtration and the resin washed and rinsed under Ar (g) with dry Et₂O. This procedure affords a reactive silyl chloride linker on Tentagel, Tent-DES. Note, the diisooronyl congener has also been fashioned similarly. Loading and Cleavage. To the rinsed and dried Tent-DES, was added dry CH₂Cl₂(20 mL). Then, 1-hydroxy-2-(1-penten4-ol)-4-methoxybenzene (69 mg, 0.33 mmol, 1.5 equiv.) was added as a CH₂Cl₂ solution, followed by freshly distilled 2,6-lutidine (51.3 μL, 0.44 mmol, 2.0 equiv.), and allowed to go overnight with occasional aggitation. After this time, the resin was washed (×8) with THF, CH₂Cl₂, MeOH, H₂O, and hexane followed by drying under high vacuum. A 500 mg sample of the resin (0.055 mmol) was placed in an epindorff tube, diluted with dry THF and a few drops of HF•pyridine were added. After 30 minutes of shaking, extraction into EtOAc and a water wash (to remove residual HF•pyridine) concentration afforded 9.9 mg (87%) yield of the parent alcohol, pure by ¹H NMR.

Supporting Information

Experimental Section

General Procedures. All reactions were performed in oven-dried glassware under a positive pressure of argon. All solid phase reactions were performed in either Chemilass solid phase reaction vessels (CG-1866) or BioRad Poly-Prep Chromratography Columns with agitation provided by a Lab-Line 3-D Rotator. All resins were first subjected to modified washing conditions of Frechet et al. (Farrall, M. J.; Frechet, J. M. J. J. Org. Chem., 1976, 41, 3877.). Loading level of the PS-DES resin was adjusted by capping with methanol, and the actual loading level was then quantified by cleavage, in triplicate, of 200 mg portions and based on average mass recovery. Flash chromatography was performed as described by Still et al. (Still, W. C.; Kahn, M.; Mitra, A. J. Org. Chem. 1978, 43, 2923.)

Materials. Tetrahydrofuran and diethyl ether were distilled under nitrogen from sodium-benzophenone ketyl. Methylene chloride, methanol and 2,6-lutidine were distilled under nitrogen form calcium hydride. Dimethylformamide, 99.9% anhydrous and iodobenzene diacetate were purchased from Aldrich. 1,3-dichloro-5,5-dimethylhydantoin was purchased from Fluka. PS-DES resin (0.76 mmol/g −0.96 mmol/g) was purchased from Argonaut Technologies.

Instrumentation. Infrared spectra were recorded on a Perkin-Elmer 1600 series FT-IR spectrometer. ¹H and ¹³C NMR were recorded on either a Bruker AM500 (500 MHz/125 MHz) or a Bruker AM400 (400 MHz/100 MHz) spectrometer. ¹H-COSY, NOE and NOESY experiments were performed on a Bruker DMX-500 spectrometer (500 MHz). Chemical shifts for proton and carbon resonances are reported in ppm (δ) relative to chloroform (δ 7.26, 77.07 respectively). HPLC data was acquired on a HP-1100 series QuatPump with a YMC S3-micron column (BA99S031046WT).

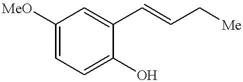

4-Methoxy-2-[E-1-propenyl]phenol

To an oven-dried 50 mL flask, equipped with stir bar and double septaed was cooled/purged under a stream of Ar(g), and was then charged with propyltriphenylphosphonium bromide (7.7 g, 20 mmol). Dry TBF (50 mL) was then added, followed by n-BuLi (8 mL, 20 mmol, 2.5 M hexanes) at room temperature to form the bright-red ylide. After 30 minutes, 2-hydroxy-5-methoxybenzaldehyde (1.25 mL, 10 mmol) was added dropwise, and was allowed to stir at room temperature for 3 hours. Upon completion, the reaction was quenched with 0.5 M HCl, extracted with EtOAc, and the organic layer was washed with water, brine and dried over anhydrous Na₂SO₄. Concentration in vacuo and column chromatography [80:20/Hex:EtOAc] afforded 1.4 g (85%) of a pale yellow oil. TLC [50:50/Hex:EtOAc] R_{f}=0.72; IR (neat, cm⁻¹): 3406, 2980, 1609 (s), 1502, 1201, 1040; ¹H NMR (500 MHz, CDCl₃): δ 6.93 (d, J=2.93 Hz, 1H), 6.71 (d, J=8.7 Hz, 1H), 6.67 (dd, J=2.9, 8.7 Hz, 1H), 6.60 (dt, J=1.5, 16 Hz, 1H), 6.25 (dt, J=6.5, 16 Hz, IH), 3.79 (s, 3H), 2.24 (m, 2H), 1.11 (t, J=7.4 Hz, 3H); ¹³C NMR (100 MHz, CDCl₃): 153.4, 146.5, 134.7, 125.8, 122.9, 116.5, 113.5, 111.8, 55.7, 26.3, 13.5; LRMS (EI+): 178 (M+), 163 (M+−Me), 137; HRMS (EI+): calculated for C₁₁H₁₄O₂, 178.0993; found 178.0991.

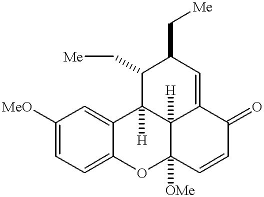

Homodimer

To an oven-dried 50 mL flask, equipped with stir bar and double septaed was cooled/purged under a stream of Ar(g), and was then charged with 4-methoxy-2-[E-1-propenyl]phenol (X)(534 mg, 3 mmol). Dry CH₂Cl₂ (20 mL, 0.15M) was then added, cooled to 0° C. and followed by iodobenzene diacetate (966 mg, 3 mmol). The solution rapidly changed from water-white to yellow, orange and finally deep red. After 10 minutes, the reaction was then diluted with CH₂Cl₂ and extacted with saturated NaHCO₃. Concentration in vacuo and column chromatography [80:20/Hex:EtOAc] afforded 493 mg (93%) of a crystalline solid. TLC [50:50/Hex:EtOAc] R_{f}=0.69; IR (neat, cm⁻¹): 3010, 2980, 1681 (s), 1617, 1494, 1202; ¹H NMR (500 MHz, CDCl₃): δ 7.21 (d, J=10.3 Hz, 1H), 7.06 (m, 1H), 6.86 (d, J=2.4 Hz, 1H), 6.7 (m, 1H), 6.6 (m, 1H), 6.29 (d, J=10.3 Hz, 1H), 3.75 (s, 3H), 3.41 (m, 1H), 3.29 (s, 3H), 3.08 (dt, J=2.6, 7.2 Hz, 1H), 2.33 (td, J=2.5, 6.2 Hz, 1H), 2.0 (m, 1H), 1.47 (m, 1H), 1.36 (m, 1H), 1.04 (t, J=7.3 Hz, 3H), 0.92 (m, 1H), 0.86 (t, J=7.2 Hz, 3H), 0.81 (m, 1H); ¹³C NMR (100 MHz, CDCl₃): δ 186.7, 154.1, 144.9, 142.8, 142.5, 131.6, 128.3, 125.4, 117.8, 113.2, 112.7, 95.7, 55.6, 49.4, 41.9, 39.6, 37.3, 32.4, 27.92, 27.9, 12.9, 12.5; MS (EI+): 354 (M+), 325 (M-Et), 256, 177 (retro-cyclo); HRMS (EI+); calculated for C₂₂H₂₆O₄, 348.1830; found. 354.1834.

Representative Procedure for the Synthesis of All Amide and Ester Coupling Components

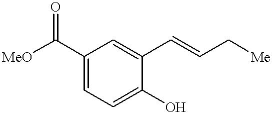

4-Carbomethoxy-2-[E-1-butenyl]phenol (X)

To an oven-dried 50 mL flask, equipped with stir bar and double septaed was cooled/purged under a stream of Ar(g), and was then charged with 5carbomethoxy-2-hydroxybenzaldehyde [Suzuki, Y.; Takahashi, H. Chem. Pharm. Bull. 1983, 31, 1751.] (3.0 g, 16.6 mmol) and dry THF (25 mL). To another oven-dried 200 mL flask, equipped with stir bar and double septaed was cooled/purged under a stream of Ar(g), and was then charged with propyltriphenylphosphonium bromide (12.8 g, 33.3 mmol) and dry THF (100 mL) [E-selective Wittig with salicylaldehydes: Jones, J. H. J. Chem Res. 1987, 3146.]. At room temperature, n-BuLi (13.3 mL, 33.3 mmol, 2.5 M hexanes) was added dropwise to the form the red, homogeneous ylide. After 30 minutes, the THF solution of 5-carbomethoxy-2-hydroxybenzaldehyde was transfered via cannula to the ylide. After 3 hours, the reaction was quenched with 0.5 M HCl, extracted into EtOAc and washed with water, brine and dried over anhydrous Na₂SO₄. Concentration in vacuo and column chromatography [80:20-50:50/Hex:EtOAc] afforded 3.1 g (90%) of a white solid. TLC [50:50/Hex:EtOAc] R_{f}=0.68; IR (neat, cm⁻¹): 3354, 2980, 1716 (s), 1682, 1601, 1274, 1131; ¹H NMR (500 MHz, CDCl₃): δ 8.06 (s, 1H), 7.77 (d, J=8.2 Hz, 1H), 6.9 (s, 1H), 6.86 (d, J=8.4 HZ, 1H), 6.60 (d, J=15.9 Hz, 1H), 6.30 (dt, J=6.5, 15.9 Hz, 1H), 3.9 (s, 3H), 2.24 (m, 2H), 1.08 (t, J=7.5 Hz, 1H); ¹³C NMR (100 MHz, CDCl₃): δ 167.8, 157.1, 135.1, 129.7, 129.1, 125.2, 122.1, 122, 115.5, 52.1, 26.3, 13.5; LRMS(CI+): 224 (M+NH₄), 207 (M+1); HRMS (CI+): calculated for C₁₂H₁₄O₃(NH₄), 224.1287; found 224.1275.

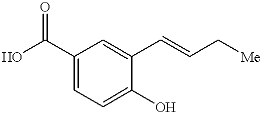

4-Carboxy-2-[E-1-butenyl]phenol (X)

To 200 mL flask, equipped with stir bar was then charged with 4-carbomethoxy-2-[E-1-butenyl]phenol (3.1 g, 15 mmol) and a 3:1 MeOH:H₂O solution (90:30 mL). At room temperature KOH (4.2 g, 75 mmol) was added, and the reaction vessel was warmed to 65° C. and allowed to stir overnight. Upon completion, the reaction was diluted with water and extracted with hexanes. The aqueous layer was then acidified with 1.0 M HCl to pH˜4, extracted into EtOAc, washed with water and brine and then dried over anhydrous Na₂SO₄. Concentration in vacuo and columnn chromatography [50:50/Hex:EtOAc] afforded 3.1 g (90%) of a white solid. TLC [50:50/Hex:EtOAc] R_{f}=0.17; IR (neat, cm⁻¹): 3360, 2980, 1710, 1633, 1204; ¹H NMR (500 MHz, DMSO-d₆): δ 12.4 (bs, 1H), 10.3 (bs, 1H), 7.91 (d, J=1.8 Hz, 1H), 7.63 (d, J=8.5 Hz, 1H), 6.86 (d, J=8.6 Hz, 1H), 6.56 (d, J=16 Hz, 1H), 6.29 (dt, J=6.5, 16 Hz, 1H), 2.17 (quint., J=7.4 Hz, 2H), 1.03 (t, J=7.4 Hz, 1H); ¹³C NMR (100 MHz, DMSO-d₆): δ 167.2, 158.2, 132.8, 129.3, 127.8, 124, 122.9, 121.5, 115.3, 25.8, 13.6; LRMS(EI+): 192 (M+), 147 (M+−CO₂); HRMS (EI+): calculated for C₁₁H₁₂O₃, 192.0787; found 192.0794.

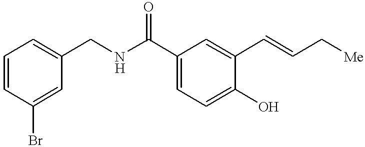

4-(meta-Bromobenzylcarboxamide)-2-[E-1-butenyl] phenol

To an oven-dried 50 mL flask, equipped with stir bar and double septaed was cooled/purged under a stream of Ar(g), and was then charged with 4-carboxy-2-[E-1-butenyl]phenol (288 mg, 1.5 mmol), PyBOP (1.17 g, 2.25 mmol), and 3-bromobenzylamine (417 mg, 2.25 mmol). Then, dry DMF (10 mL) was added followed by cooling to 0° C. Next, diisopropylethylamine (0.80 mL, 4.5 mmol) was added, and the reaction was allowed to slowly warm to room temperature overnight. The reaction was quenched with 0.5 M HCl, extracted into EtOAc, washed with water, brine, and dried over anhydrous Na₂SO₄. Concentration in vacuo and column chromatography [50:50/Hex:EtOAc] afforded 436 mg (81%) of a white foam. TLC [50:50/Hex:EtOAc] R_{f}0.26; IR (neat, cm⁻¹):3376, 2978, 1686, 1500; ¹H NMR (400 MHz, CDCl₃): δ 7.77 (d, J=2.1 Hz, 1H), 7.62 (s, 1H), 7.45 (m, 2H), 7.36 (m, 1H), 7.24 (m, 1H), 7.16 (m, 1H), 6.8 (d, J=8.4 Hz, 1H), 6.65 (t, J=5.7 Hz, 1H), 6.57 (d, J=16 Hz, 1H), 6.25 (dt, J=6.4, 16 Hz, 1H), 4.56 (d, J=5.8 Hz, 2H), 2.2 (quint., J=7.7 Hz, 2H), 1.05 (t, J=7.7 Hz, 3H); ¹³C NMR (100 MHz, CDCl₃): δ 167.9, 156.5, 140.5, 135.5, 130.7, 130.6, 130.3, 126.9, 126.4, 126.2, 125.4, 122.7, 122.5, 115.8, 43.5, 26.4, 13.6; LRMS(TOF, ES+): 362 (M+⁸¹Br), 360 (M+⁷⁹Br); HRMS(TOF, ES+): calculated for C₁₈H₁₈NO₂Br, 360.0599; found 360.0609.

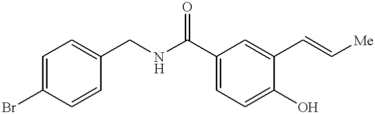

4-(para-Bromobenzylcarboxamide)-2-[E-1-propenyl]phenol

To an oven-dried 200 mL flask, equipped with stir bar and double septaed was cooled/purged under a stream of Ar(g), and was then charged with 4-carboxy-2-[E-1-propenyl]phenol (3.38 g, 18.9 mmol), PyBOP (19.7 g, 37.9 mmol), and 4-bromobenzylamine (6.38 g, 28.4 mmol). Then, dry DMF (20 mL)/CH₂Cl₂ (30 mL) were added followed by cooling to 0° C. Next, diisopropylethylamine (9.8 mL, 57 mmol) was added, and the reaction was allowed to slowly warm to room temperature overnight. The reaction was quenched with 0.5 M HCl, extracted into EtOAc, washed with water, brine, and dried over anhydrous Na₂SO₄. Concentration in vactio and column chromatography [50:50/Hex:EtOAc] afforded 5.4 g (84%) of a yellow foam. TLC [50:50/Hex:EtOAc] R_{f}=0.26; IR (neat, cm⁻¹): 3401, 2982, 1684, 1500; ¹H NMR (400 MHz, CDCl₃): δ 7.73 (d, J=2.2 Hz, 1H), 7.61 (bs, 1H), 7.45 (d, J=2.2 Hz, 1H), 7.41 (d, J=8.3 Hz, 2H), 7.17 (d, J=8.3 Hz, 2H), 6.77 (d, J=8.4 Hz, 1H), 6.61 (t, J=5.7 Hz, 1H), 6.57 (dd, J=1.6, 16Hz, 1H), 6.22 (dq, J=1.6, 16Hz, 1H), 4.53 (d, J=5.8 Hz, 2H), 1.84 (dd, J=1.6, 6.6 Hz, 3H); ¹³C NMR (100 MHz, CDCl₃): δ 167.8, 156.4, 137.1, 131.7, 129.4, 128.7, 126.7, 126.2, 125.4, 124.7, 121.4, 115.8, 34.4, 18.8; LRMS(TOF, ES+): 348 (M+⁸¹Br), 346 (M+⁷⁹Br); HRMS(TOF, ES+): calculated for C₁₇H₁₆NO₂Br, 346.0442; found 346.0451.

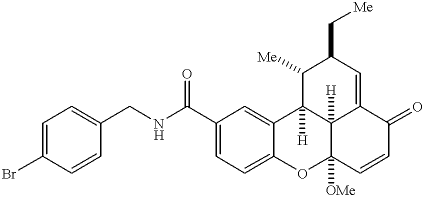

Heterodimer (X)

To an oven-dried 25 mL flask, equipped with stir bar and double septaed was cooled/purged under a stream of Ar(g), and was then charged with 4-methoxy-2 -[E-1-propenyl]phenol (X) (382 mg, 2.15 mmol) and 4-(para-bromobenzylcarboxamide)-2 -[E-1-propenyl]phenol (X) (150 mg, 0.43 mmol). Dry CH₂Cl₂ (10 mL, 0.2M) was then added, followed by iodobenzene diacetate (692 mg, 2.15 mmol). The solution rapidly changed from water-white to yellow, orange and finally deep red. The reaction was then diluted with CH₂Cl₂ and washed with saturated NAHCO₃. Concentration in vacuo and column chromatography [80:20-50:50/ Hex:EtOAC] afforded 157 mg (70%) of an off-white foam along with 263 mg 69%) of the homodimer. TLC [50:50/Hex:EtOAc] R_{f}=0.23; IR (neat, cm⁻¹): 3010, 2980, 1680 (s), 1636, 1539, 1488, 1267, 1209; ¹H NMR (500 MHz, CDCl₃): δ 7.93 (s, 1H), 7.46 (m, 3H), 7.23 (m, 4H), 7.1 (m, 1H), 6.83 (d, J=8.4 Hz, 1H), 6.34 (d, J=10.3 Hz, 1H), 6.3 (t, J=5.6 Hz, 1H), 4.59 (abquart. J=5.9, 17.7 Hz, 2H), 3.35 (m, 1H), 3.32 (dt J=2.6, 7.2 Hz, 1H), 3.31 (s, 3H), 2.8 (m, 1H), 1.92 (m, 1H), 1.15 (d, J=7.1 Hz, 3H), 0.80 (m, 5H); ¹³C NMR (100 MHz, CDCl₃): δ 186, 166, 153, 142, 141, 137, 132, 131, 129, 128, 127.6, 127.3, 125.6, 125.1, 121, 117, 96, 49, 43.9, 43.3, 36.9, 33.4, 32.4, 28, 21, 12; MS (FAB+): 544 (M+Na), 522 (M+), 441, 347; HRMS(FAB+); calculated for C₁₈H₂₈O₄N⁸¹Br, 524.1306 and C₂₈H₂₈O₄N⁷⁹Br, 522.1280; found. 524.1306 and 522.1280, respectively.

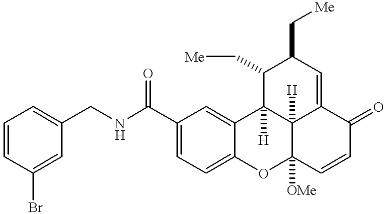

Heterodimer (X)

To an oven-dried 25 mL flask, equipped with stir bar and double septaed was cooled/purged under a stream of Ar(g), and was then charged with 4-methoxy-2 -[E-1-propenyl]phenol (X) (382 mg, 2.15 mmol) and 4-(meta-bromobenzylcarboxamide)-2 -[E-1-butenyl]phenol (X) (155 mg, 0.43 mmol). Dry CH₂Cl₂ (10 mL, 0.2M) was then added, followed by iodobenzene diacetate (692 mg, 2.15 mmol). The solution rapidly changed from water-white to yellow, orange and finally deep red. The reaction was then diluted with CH₂Cl₂ and extacted with saturated NaHCO₃. Concentration in vacuo and column chromatography [80:20-50:50/Hex:EtOAc] afforded 180 mg (75%) of an off-white foam along with 265 mg (70%) of the homodimer. TLC [50:50/Hex:EtOAc] R_{f}=0.21; IR (neat, cm⁻¹): 3010, 2980, 1680 (s), 1636, 1539, 1488, 1267, 1209; ¹H NMR (500 MHz, CDCl₃): δ 7.94 (s, 1H), 7.56 (dd, J=1.1, 7.9 Hz, 1H), 7.46 (dd, J=1.6, 7.9 Hz, 2H), 7.3 (td, J=1.1, 7.5 Hz, 1H), 7.23 (d, J=10.3 Hz, 1H), 7.16 (td, J=1.6, 7.7 Hz, 1H), 7.07 (m, 1H), 6.82 (d, J=8.4 Hz, 1H), 6.57 (t, J=5.6 Hz, 1H), 6.32 (d, J=10.3 Hz, 1H), 4.69 (abquart., J=6.1, 21 Hz, 2H), 3.47 (m, 1H), 3.30 (s, 3H), 3.13 (dt, J=2.6, 7.2 Hz, 1H), 2.44 (m, 1H), 1.48 (m, 1H),1.37 (m, 1H), 1.04 (t, J=7.3 Hz, 3H), 0.83 (m, 4H) 0.70 (m, 1H); ¹³C NMR (100 MHz, CDCl₃): δ 186.3, 166.8, 153.9, 143.2, 141.7, 137.3, 132.8, 131.9, 129.2, 128.2, 127.8, 127.7, 127.4,125.5, 125.2 123.8, 117.2, 105.7, 96, 49.3, 44.3, 41.9, 39.5, 37.1, 31.9, 29.6, 28.1, 27.9, 12.8, 12.4; MS (FAB+): 560 (M+Na, ⁸¹Br), 558 (M+Na, ⁷⁹Br) 538 (M+), 456 (M−Br), 351, 329; HRMS (FAB+); calculated for C₂₉H₃₀O₄NBr(Na), 558.1256; found 558.1271.

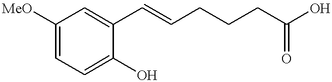

4-Methoxy-2-[6-carboxy-E-1-hexenyl]phenol (X)

To an oven-dried 200 mL flask, equipped with stir bar and double septaed was cooled/purged under a stream of Ar(g), and was then charged with 4-carboxybutyltriphenylphosphonium bromide (8.86 g, 20 mmol). Dry THF (60 mL) was then added, and the reaction vessel was cooled to 0° C. Then, two equivalents of freshly prepared LiN(TMS)₂ (40 mL, 40 mmol, 1.0 M) were added via syringe and was allowed to stir at 0° C. for 30 minutes to generate the ylide-carboxylate. In another oven-dried 100 mL flask, equipped with stir bar and double septaed was cooled/purged under a stream of Ar(g), and was then charged with 2-hydroxy-5-methoxybenzaldehyde (2.5 mL, 20 mmol). Dry TBF (40 mL) was added, and the reaction vessel was cooled to 0° C. Then, one equivalent of freshly prepared LiN(TMS)₂ (20 mL, 20 mmol, 1.0 M) was added via syringe and was allowed to stir at 0° C. for 30 minutes to generate the phenoxide. After this time, the phenoxide was transfered via cannula to the ylide-carboxylate and allowed to slowly warm to room temperature over 3 hours. Upon completion, the reaction was quenched with water (100 mL), and extracted with hexanes. The aqueous layer was then acidified to pH-4 with 0.5 M HCl, and extracted into EtOAc. The organic layers were then washed with water, brine and dried over anhydrous Na₂SO₄. Concentration in vacuo and column chromatography [40:60/Hex:EtOAc] afforded 4.0 g (85%) of a yellow-green viscous oil. TLC [50:50/Hex:EtOAc] R_{f}=0.05; IR (neat, cm⁻¹): 3361, 2939, 1701 (s), 1505, 1202; ¹H NMR (500 MHz, DMSO-d₆): δ 11.9 (vbs, 1H), 9.01 (bs, 1H), 6.9 (d, J=2.98 Hz, 1H), 6.70 (d, J=8.83 Hz, 1H), 6.60 (dd, J=3, 8.8 Hz, 1H), 6.56 (d, J=16 Hz, 1H), 6.19 (dt, J=6.9, 16 Hz, 1H), 3.65 (s, 3H), 2.24 (t, J=7.4 Hz, 2H), 2.16 (q, J=7.3 Hz, 2H), 1.64 (m, 2H); ¹³C NMR (500 MHz, DMSO-d₆): δ 174.4, 152.2, 148.1, 129.4, 125.1, 124.4, 116.3, 113.7, 110.5, 55.3, 33.1, 32.2, 24.3; LRMS(EI+): 236 (M+), 218 (M−OH), 190, 163; HRMS(EI+); calculated for C₁₃H₁₆O₄, 236.1049; found 236.1052.

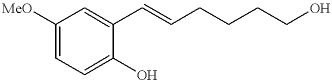

4-Methoxy-2-[6-hydroxy-E-1-hexenyl]phenol (X)

To an oven-dried 100 mL flask, equipped with stir bar and double septaed was cooled/purged under a stream of Ar(g), and was then charged with 4-methoxy-2-[6-carboxy-E-1-hexenyl]phenol (X) (1.2 g, 5.08 mmol). Dry THF (30 mL, 0.16 M) was then added, and the reaction vessel was cooled to −78° C. Then, LiAlH₄ (10.16 mL, 10.16 mmol, 1.0M THF) was added dropwise via syringe. The reaction was allowed to warm slowly to room temperature and the reaction progress was monitored by TLC. Upon completion, a saturated solution of Rochelle's salt was added and stirred until the organic layer was clear and homogeneous. The mixture was then extracted into EtOAc, washed with water, brine and dried over anhydrous Na₂SO₄. Concentration in vacuo and column chromatography [40:60/Hex:EtOAc] afforded 1.06 g (94%) of a yellow oil. TLC [50:50/Hex:EtOAc] R_{f}=0.53; IR (neat, cm⁻¹): 3406, 2980, 1609, 1502, 1040; ¹H NMR (500 MHz, CDCl₃): δ 6.87 (d, J=2.8 Hz, 1H), 6.7 (d, J=8 Hz, 1H), 6.63 (dd, J=2.8, 8 Hz, 1H), 6.58 (d, J=16 Hz, 1H), 6.16 (dt, J=6.3, 16 Hz, 1H), 3.75 (s, 3H), 3.66 (t, J=6 Hz, 2H), 2.23 (q, J=7.1 Hz, 2H), 1.62 (m, 2H), 1.53 (m, 2H); ¹³C NMR (100 MHz, CDCl₃): δ 153.6, 146.9, 132.5, 125.7, 124.5, 116.6, 113.6, 111.8, 62.8, 55.8, 33, 32.1, 25.4; LRMS(EI+): 223 (M+1), 222 (M+); HRMS(EI+); calculated for C₁₃H₁₈O₂, 222.1256; found 222.1250.

[chemical structure]

4-Methoxy-2-(6-PS-DESsilyloxy-E-1-hexenyl]phenol (X)

To a dry Schlenk flask was placed the commercially available PS-DES resin, and subjected to a modification of the Frechet washing procedure. At 45° C., the resin was suspended and washed with water (30 minutes), DMF (30 minutes), TEF (30 minutes), and finally MeOH:CH₂Cl₂ (1:3). The resin was then washed with dry hexanes and placed under high vacuum for 5 hours. To an oven-dried Chemglass solid phase reaction vessel, cooled/purged under a stream of argon was placed the washed/dried PS-DES resin (1 g, 0.96 mmol, 0.96 mmol/g) and 1,3-dichloro-5,5-dimethylhydantoin (567 mg, 2.88 mmol). Dry CH₂Cl₂ (15 mL) was then added, and the reaction vessel was placed on an orbital stirrer and agitated at room temperature for 2 hours. After this time, the resin was filtered under argon and washed with dry THI (3×80 mL) and CH₂Cl₂ (3×80 mL) to remove the excess 1,3-dichloro-5,5-dimethylhydantoin. The resin was then re-swollen in cold CH₂Cl₂ (20 mL). In an oven-dried flask, cooled/purged under argon, was placed 4-methoxy-2-[6-hydroxy-E-1-hexenyl]phenol (634 μL, 0.32 mmol, 0.5 M CH₂Cl₂), dry CH₂Cl₂ (5 mL) and freshly distilled MeOH (26 μL, 0.63 mmol). This 0° C. solution was then transfered via cannula to the resin, followed immediately by a 0° C. CH₂Cl₂ solution of freshly distilled 2,6-lutidine (116 μL, 1.0 mmol). [Note: 2,6-lutidine is required to selctively silylate the alcohol moiety over the phenol.] The reaction vessel was again placed on the orbital stirrer and allowed to stir at room temperature for 18 hours. After this time, the resin was washed (×8): CH₂Cl₂, THF, MeCN, DMF, MeOH, H₂O, hexane and dried in vacuo. The loading level was calculated to be 0.24 mmol/g (based on MeOH added as a capping reagent). The actual loading level was determined by placing PS-DES (3×100 mg) in 10 mL BioRad tubes, diluting with THF (0.5 mL) and treatment with HF•pyridine (50 μL) for 2 hours on the orbital stirrer. After this time, TMSOMe (0.5 mL) was added, the reaction let stir 20 minutes,. After this time, the resin was filtered and washed with CH₂Cl₂ to afford a white solid upon concentration. Column chromatography [9:1/CH₂Cl₂:MeOH] afforded 5 mg (89%), 5.2 mg (93%) and 5.2 mg (93%) of white solid. Therefore the loading level was determined to be 0.22 mmol/g. TLC [50:50/Hex:EtOAc] R_{f}=0.53; IR (neat, cm⁻¹): 3406, 2980, 1609, 1502, 1040; ¹H NMR (500 MHz, CDCl₃): δ 6.87 (d, J=2.8 Hz, 1H), 6.7 (d, J=8 Hz, 1H), 6.63 (dd, J=2.8, 8 Hz, 1H), 6.58 (d, J=16 Hz, 1H), 6.16 (dt, J=6.3, 16 Hz, 1H), 3.75 (s, 3H), 3.66 (t, J=6 Hz, 2H), 2.23 (q, J=7.1 Hz, 2H), 1.62 (m, 2H), 1.53 (m, 2H); ¹³C NMR (100 MHz, CDCl₃): δ 153.6, 146.9, 132.5, 125.7, 124.5, 116.6, 113.6, 111.8, 62.8, 55.8, 33, 32.1, 25.4;; LRMS(EI+): 223 (M+1), 222 (M+); HRMS(EI+); calculated for C₁₃H₁₈O₂, 222.1256; found 222.1250.

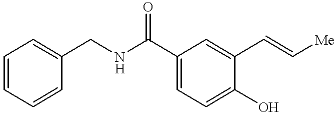

4-(Benzylcarboxamide)-2-[E-1-propenyl]phenol (X)

To an oven-dried 200 mL flask, equipped with stir bar and double septaed was cooled/purged under a stream of Ar(g), and was then charged with 4-(carboxy)-2-[E-1-propenyl]phenol (X) (2.48 g, 13.9 mmol), EDC (5.3 g, 27.8 mmol), HOBt (2.82 g, 20.9 mmol) and purged with argon. Dry DMF (70 mL) was then added, followed by benzylamine (2.28 mL, 20.9 mmol) and freshly distilled diisopropylethylamine (7.22 mL, 41.7 mmol). The reaction was allowed to stir overnight at room temperature. Upon completion, the reaction was diluted with EtOAc and washed with 0.5 M HCl, water, brine and dried over anhydrous Na₂SO₄. Concentration in vacuo and column chromatography [50:50Hex:EtOAc] afforded 3.06 g (81%) of an off-white solid. TLC [50:50/Hex:EtOAc] R_{f}=0.29; IR(neat, cm⁻¹): 3380, 2979, 1701, 1686, 1500; ¹H NMR (500 MHz, DMSO-d₆): δ 10.0 (bs, 1H), 8.80 (t, J=5.9 Hz, 1H), 7.9 (d, J=2.1 Hz, 1H), 7.29 (s, 4H), 7.23 (m, 1H), 6.83 (d, J=8.4 Hz, 1H), 6.6 (dd, J=1.6, 15.9 Hz, 1H), 6.3 (dq, J=6.7, 15.9 Hz, 1H), 4.43 (d, J=8 Hz, 2H), 1.84 (dd, J=1.6, 16Hz, 3H); ¹³C NMR (100 MHz, DMSO-d₆): δ 185.6, 156.6, 140, 128.2, 127.3, 127.1, 126.6, 125.7, 125.4, 125.1, 123.8, 115.1, 42.4, 18.6; LRMS (FAB+): 290 (M+Na), 268 (M+); HRMS (FAB+): calculated for C₁₇H₁₇NO₂(Na), 290.1157; found 290.1165.

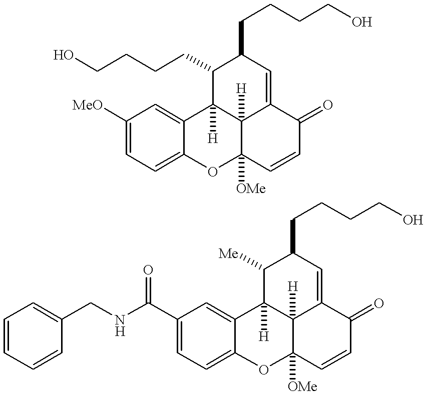

Solid Phase (silyl ether) Hetero/Homo

To a 20 mL BioRad tube was placed resin (X) (750 mg, 0.165 mmol, 0.22 mmol/g) and 4-(benzylcarboxamide)-2-[E-1-propenyl]phenol (X) (440.5 mg, 1.65 nimol, 10.0 equiv.). Dry CH₂Cl₂ (8 mL) and THF (2.5 mL) were added to swell the resin and dissolve the amide. Then, the BioRad tube was placed on an orbital stirrer and allowed to stir for 30 minutes to afford good mixing. After this time, IPh(OAc)₂ (531 mg, 1.65 mmol, 10.0 equiv.) was added in one portion, the tube was shaken vigorously, then placed on an orbital stirrer and agitated for 2 hours. During this time, the resin/solution darkened to a deep orange hue. After this time, the tube was attached to a Promega wash station, and the resin washed (×8); CH₂Cl₂, 1% Et₃N/CH₂Cl₂, THF, MeOH, H₂O, CH₃CN and then dried. The resin was then transfered into another 20 mL BioRad tube, swollen in THF (5 mL) and HF•pyridine (400 μL) was added. Again, the tube placed on an orbital stirrer for 2 hours. Then, TMSOMe (1.5 niL) was added, and allowed to stir for an addition 2 hours. After this time, the resin was filtered and washed with CH₂Cl₂ to afford a yellow film upon concentration. Column chromatography [75:25/Hex:EtOAc—9:1/CH₂Cl₂:MeOH] afforded 39 mg (49%) of heterodimer as a colorless film (TLC [75:25/Hex:EtOAc] R_{f}=0.41) along with 10.3 mg (29%) of homodimer as a colorless film (TLC [50:50/Hex:EtOAc] R_{f =0.05}), [Heterodimer] IR(neat, cm⁻¹): 3510, 3009, 2980, 1680, 1635, 1539, 1480, 1267, 1209; ¹H NMR (500 MHz, CDCl₃): δ 8.01 (s, 1H), 7.4 (d, J=8.3 Hz, 1H), 7.35 (s, 4H), 7.29 (m, 1H), 7.23 (d, J=10.4 Hz, 1H), 7.03 (m, 1H), 6.83 (d, J=8.4 Hz, 1H), 6.38 (t, J=5.7 Hz, 1H), 6.35 (d, J=10.4 Hz, 1H), 4.63 (m, 2H), 3.48 (m, 2H), 3.42 (m, 1H), 3.37 (m, 1H), 3.31 (s, 3H), 3.21 (dt, J=5.5, 9.7 Hz, 1H), 2.92 (m, 1H), 2.02 (m, 1H), 1.69 (m, 2H), 1.34 (m , 2H), 1.23 (m, 1H), 1.15 (d, J=7.1 Hz, 3H), 0.87 (m, 1H), 0.41 (m, 1H); ¹³C NMR (100 MHz, CDCl₃): δ 186.2, 168.3, 153.8, 143.2, 141.5, 138.2, 132, 128.7, 127.9, 127.6, 125.56, 127.51, 125.3, 125.2, 117.2, 96.2, 62, 49.3, 44.1, 42.2, 36.8, 33.8, 33.4, 32.5, 30.7, 24.1, 21.7; LRMS (TOF, ES+): 489 (M+1), 488 (M+), 381; HRMS (TOF, ES+): calculated for C₃₀H₃₃NO₅, 488.2437; found 488.2451; [Homodimer] IR(neat, cm⁻¹): 3450, 3010, 2978, 1680, 1616, 1490; ¹H NMR (500 MHz, CDCl₃): δ 7.22 (d, J=10.3 Hz, 1H), 6.99 (m, 1H), 6.88 (d, J=2.6 Hz, 1H), 6.71 (d, J=8.8 Hz, 1H), 6.65 (dd, J=2.8, 8.8 Hz, 1H), 6.28 (d, J=10.3 Hz, 1H), 3.74 (s, 3H), 3.63 (t, J=6.18 Hz, 2H), 3.45 (td, J=1.3, 6.18 Hz, 2H), 3.39 (d, J=7.1 Hz, 1H), 3.27 (s, 3H), 3.07 n(dt, J=2.45, 7.2 Hz, 1H), 2.48 (m, 1H), 2.1 (m, 1H), 2.06 (bs, 2H), 1.59 (m, 2H), 1.49 (m, 4H), 1.39 (m, 2H), 1.28 (m, 2H), 0.96 (m, 1H), 0.63 (m, 1H); ); ¹³C NMR (125 MHz, CDCl₃): δ 186.6, 153.9, 144.9, 142.9, 142.5, 131.5, 128.3, 125.5, 117.8, 113.3, 112.6, 95.6, 62.5, 62, 55.7, 49.1, 40.1, 37.3, 37, 34.8, 33.8, 32.6, 32.3, 24.1, 23.9; LRMS (TOF, ES+): 444 (M+1), 443 (M+), 411 (M+−OMe), 301, 221 (retro-cyclo); HRMS (TOF, ES+): calculated for C₂₆H₃₃O₆, 443.2433; found 443.2411.

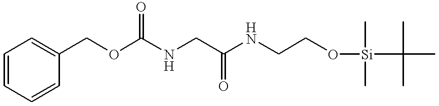

N-α-Benzyloxycarbonylglycine-N-(2-t-butyldimethylsilyloxy-1-ethyl)amide (X)

To an oven-dried 200 mL flask, equipped with stir bar and double septaed was cooled/purged under a stream of Ar(g), and was then charged with Z-Gly-OH (4.18 g, 20 mmol), EDC (5.72 g, 30 mmol), HOBt (2.7 g, 20 mmol) and purged with argon. Dry DMF (40 mL) was then added, followed by ethanolamine (3.6 mL, 60 mmol). The reaction was allowed to stir overnight at room temperature. After this time, TBDMSCl (12g, 80 mmol) and imidazole (5.4 g, 80 mmol) were added in dry CH₂Cl₂ (100 mL) via cannula to the crude coupling reaction at 0° C. Upon completion, the reaction was diluted with EtOAc and washed with 0.5 M HCl, water, brine and dried over anhydrous Na₂SO₄. Concentration in vacuo and column chromatography [50:50/Hex:EtOAc] afforded 7.19 g (98%) of an off-white solid. TLC [50:50tHex:EtOAc] R_{f}=0.12; IR (neat, cm⁻¹): 2980, 2870, 1717 (s), 1663, 1533; ¹H NMR (500 MHz, CDCl₃): δ 7.32 (m, 5H), 6.2 (bs, 1H), 5.3 (bs, 1H), 5.1 (s, 1H), 3.87 (d, J=5.4 Hz, 2H), 3.66 (m, 2H), 3.39 (q, J=5.2 Hz, 2H), 0.88 (s, 9H), 0.05 (s, 6H); ¹³C NMR (100 MHz, CDCl₃): δ 168.9, 156.4, 136, 128.3, 127.9, 66.9, 61.4, 44.4, 41.5, 25.7, 18, −5.5; LRMS (FAB+): 384 (M+NH₄), 367 (M+1), 221; HRMS (CI+): calculated for C₁₈H₃₀O₄N₂Si (NH₄), 384.2319; found 384.2305.

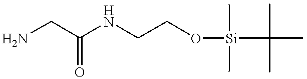

N-α-H-N-(2-t-butyldimethylsilyloxy-1-ethyl)amide

To an oven-dried 200 mL flask, equipped with stir bar and double septaed was cooled/purged under a stream of Ar(g), and was then charged with N-α-benzyloxycarbonylglycine-N-(2-t-butyldimethylsilyloxy-1 -ethyl)amide (X) (7.19 g, 20 mmol). EtOH (80 mL) was then added, followed by a catalytic amount of 10% palladium/carbon. The flask was purged/evacuated with H₂(g) from a balloon (×3), and then allowed to stir under an H₂ atmosphere for 18 hours. Filtration of the solution through a sintered-glass frit atop a pad of celite and concentration in vacuo afforded 4.4 g (98%) of a water-white oil. TLC [EtOAc] R_{f}=0.16; IR (neat, cm⁻¹): 3408, 2980, 2870, 1710, 1530, 1250; ¹H NMR (500 MHz, CDCl₃): δ 7.54 (bs, 1H), 3.69 (t, J=5.3 Hz, 2H), 3.4 (q, J=5.4 Hz, 2H), 3.36 (bs, 2H), 1.54 (bs, 2H), 0.89 (s, 9H), 0.09 (s, 6H); ¹³C NMR (100 MHz, CDCl₃): δ 169.9, 61.6, 43.1, 41.4, 25.8, 18.1, −5.3; LRMS (FAB+): 255 (M+Na), 233 (M+1), 232 (M+); HRMS (FAB+): calculated for C₁₀H₂₄N₂O₂Si(Na), 255.1505; found 255.1502.

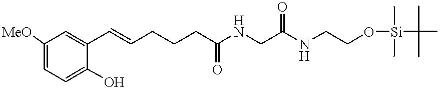

To an oven-dried 200 mL flask, equipped with stir bar and double septaed was cooled/purged under a stream of Ar(g), and was then charged with N-α-H-N-(2 -t-butyldimethylsilyloxy-1-ethyl)amide (X) (4.4 g, 19 mmol), EDC (5.46 g, 28.6 mmol), HOBt (2.12 g, 15.7 mmol) and purged with argon. Dry DMF (60 mL) was then added, followed by freshly distilled diisopropylethylamine (7.42 mL, 42.9 mmol) at 0° C. The reaction was allowed to stir for 30 minutes to neutralize the HCl. After this time, 4-methoxy-2-[6-carboxy-E-1-hexenyl]phenol (X) (3.38 g, 14.3 mmol) was dissolved in dry DMF (40 mL) and transferred via cannula to the amine at 0° C. The reaction was then allowed to slowly wvarm to room temperature overnight. Upon completion, the reaction was diluted with EtOAc and washed with 0.5 M HCl, water, brine and dried over anhydrous Na₂SO₄. Concentration in vacuo and column chromatography [50:50/Hex:EtOAc] afforded 5.9 g (92%) of a white solid. TLC [50:50/Hex:EtOAc] R_{f}=0.19; IR (Neat, cm⁻¹): 3299, 1650, 1537, 1504, 1428, 1207, 1104, 779; ¹H NMR (500 MHz, CDCl₃): δ 6.86 (d. J=3 Hz, 2H), 6.38 (bs, 1H), 6.75 (d, J=8.7 Hz, 1H), 6.64 (m, 1H), 6.57 (t, J=5 Hz. 1H). 6.47 (t, J=5 Hz, 1H), 6.06 (dt, J=7, 15 Hz, 1H), 3.9 (d, J=5 Hz, 2H), 3.75 (s, 3H), 3.66 (t, J=5 Hz, 2H), 3.37 (q, J=5.4 Hz, 2H), 2.25 (m, 2H), 1.82 (M, 2H), 0.88 (S, 9H), 0.05 (S, 6H); ¹³C NMR (100 MHz, CDCl₃): 173.8, 169.3, 153.4, 147.4, 130.6, 125.9, 125.5. 117, 113.8, 111.4, 61.5, 55.7, 43, 41.8, 35.1, 32.4, 25.8, 24.5, 18.2, −5.4; LRMS (TOF, ES+): 451 (M+); HRMS (TOF, ES+): calculated for C₂₃H₃₈N₂O₅Si; 451.2628: found 451.2614.

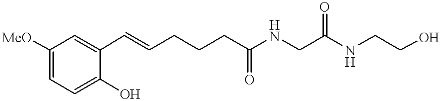

Something (X)

To a polyethylene bottle equipped with stir bar was charged with something (X) (5.9 g, 13.1 mmol), followed by freshly distilled THF (30 mL). The reaction vessel was cooled to 0° C., whereupon HF•pyridine (1.5 mL) was added and the reaction monitored by TLC. Upon completion, the reaction was diluted with EtOAc and washed with 0.5 M HCl, water, brine and dried over anhydrous Na₂SO₄. Concentration in vacuo and column chromatography [9:1/CH₂Cl₂:MeOH] afforded 4.3 g (95%) of an off-white solid. TLC [9:1/CH₂Cl₂:MeOH] R_{f}=0.10; ¹H NMR (500 MHz, DMSO-d₆): δ 9.0 (s, 1H), 8.01 (t, J=5.7 Hz, 1H), 7.77 (t, J=5.7 Hz, 1H), 6.9 (d, J=3 Hz, 1H), 6.7 (d, J=8.7 Hz, 1H), 6.59 (m, 2H), 6.21 (dt, J=7, 15 Hz, 1H), 4.65 (t, J=5.4 Hz, 2H), 3.66 (s, 3H), 3.64 (m, 2H), 3.37 (s, 2H), 3.37 (m, 2H), 3.11 (m, 2H), 2.16 (m, 4H), 1.65 (m, 2H); ); ¹³C NMR (100 MHz, DMSO-d₆): δ 172.4, 169.1, 152.2, 148.1, 129.7, 124.9, 124.5, 116.3, 113.6, 110.6, 59.7, 55.3, 42, 41.4, 34.7, 32.4, 25; LRMS (FAB+): 359 (M+Na), 329, 273; HRMS(TOF, ES+): calculated for C₁₇H₂₄O₅N₂(Na), 359.1583; found 359.1594 (M+Na) and 337.1779 (M+).

[chemical structure]

PS-DES (X)

To a dry Schlenk flask was placed the commercially available PS-DES resin, and subjected to a modification of the Frechet washing procedure. At 45° C., the resin was suspended and washed with water (30 minutes), DMF (30 minutes), THF (30 minutes), and finally MeOH:CH₂Cl₂ (1:3). The resin was then washed with dry hexanes and placed under high vacuum for 5 hours. To an oven-dried Chemglass solid phase reaction vessel, cooled/purged under a stream of argon was placed the washed/dried PS-DES resin (2 g, 0.96 mmol, 1.92 mmol/g) and 1,3-dichloro-5,5-dimethylhydantoin (1.13 mg, 5.76 mmol). Dry CH₂Cl₂ (30 mL) was then added, and the reaction vessel was placed on an orbital stirrer and agitated at room temperature for 2 hours. After this time, the resin was filtered under argon and washed with dry THF (3×80 mL) and CH₂Cl₂ (3×80 mL) to remove the excess 1,3-dichloro-5,5-dimethylhydantoin. The resin was then re-swollen in cold CH₂Cl₂ (20 mL). In an oven-dried flask, cooled/purged under argon, was placed something (X) (161.3 mg, 0.48 mmol), dry CH₂Cl₂ (5 mL), dry DMF (2 mL) and freshly distilled MeOH (58.5 μL, 1.44 mmol). This 0° C. solution was then transfered via cannula to the resin, followed immediately by a 0° C. CH₂Cl₂ solution of freshly distilled 2,6-lutidine (233 μL, 2.0 mmol). [Note: 2,6-lutidine is required to selctively silylate the alcohol moiety over the phenol.] The reaction vessel was again placed on the orbital stirrer and allowed to stir at room temperature for 36 hours. After this time, the resin was washed (×8): CH₂Cl₂, THF, MeCN, DMF, MeOH, H₂O, hexane and dried in vacuo. The loading level was calculated to be 0.24 mmol/g (based on MeOH added as a capping reagent). The actual loading level was deteremined by placing PS-DES (3×200 mg) in 10 mL BioRad tubes, diluting with THF (0.5 mL) and treatment with HF•pyridine (50 μL) for 2 hours on an orbital stirrer. After this time, TMSOMe (0.5 mL) was added, the reaction let stir 2 more hours. After this time, the resin was filtered and washed with CH₂Cl₂ to afford a white solid upon concentration. Column chromatography [9:1/CH₂Cl₂:MeOH] afforded 12.5 mg (79%), 12 mg (75%) and 12.3 mg (77%) of white solid. Therefore the loading level was determined to be 0.19 mmol/g. TLC [9:1/CH₂Cl₂:MeOH] R_{f}=0.10; ¹H NMR (500 MHz, DMSO-d₆): δ 9.0 (s, 1H), 8.01 (t, J=5.7 Hz, 1H), 7.77 (t, J=5.7 Hz, 1H), 6.9 (d, J=3 Hz, 1H), 6.7 (d, J=8.7 Hz, 1H), 6.59 (m, 2H), 6.21 (dt, J=7, 15 Hz, 1H), 4.65 (t, J=5.4 Hz, 2H), 3.66 (s, 3H), 3.64 (m, 2H), 3.37 (s, 2H), 3.37 (m, 2H), 3.11 (m, 2H), 2.16 (m, 4H), 1.65 (m, 2H); ); ¹³C NMR (100 MHz, DMSO-d₆): δ 172.4, 169.1, 152.2, 148.1, 129.7, 124.9, 124.5, 116.3, 113.6, 110.6, 59.7, 55.3, 42, 41.4, 34.7, 32.4, 25; LRMS (FAB+): 359 (M+Na), 329 ,273; HRMS(TOF, ES+): calculated for C₁₇H₂₄O₅N₂(Na), 359.1583; found 359.1594 (M+Na) and 337.1779 (M+).

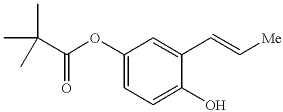

4-Pivoyl-2-[E-1-propenyl]phenol

To an oven-dried 25 mL flask, equipped with stir bar and double septaed was cooled/purged under a stream of Ar(g), and was then charged with 2,5-dihydroxybenzaldehyde (1 g, 7.2 mmol). Dry TBF (40 mL, 0.18M) was then added, and the reaction vessel was cooled to −78° C. whereupon NaN(TMS)₂ (14.4 mL, 14.4 mmol, 1.0 M THF) was added dropwise to form the diphenoxide. After 20 minutes, pivaloyl chloride (885 μL, 7.2 mmol) was added dropwise, and the reaction was allowed to slowly warmn to room temperature. Extractive work-up provided the monopivolate (810 mg, 51%). To an oven-dried 25 mL flask, equipped with stir bar and double septaed was cooled/purged under a stream of Ar(g), and was then charged with the ethyltriphenylphosphonium bromide (2.71 g, 7.3 mmol) and dry THE (30 mL, 0.24 M). At room temperature, n-BuLi (2.92 mL, 7.3 mmol, 2.5 M hexanes) was added dropwise forming the red, homogeneous ylide. At this time, the monopivolate (810 mg, 3.64 mmol) was added to the ylide via cannula as a THF solution (10 mL) and allowed to stir at room temperature for 3 hours. Aqueous work-up and extraction inot Et₂O, followed by concentration in vacuo and column chromatography [80:20-50:50/Hex:EtOAc] afforded 683 mg (80%) of a white solid. TLC [50:50/Hex:EtOAc] R_{f}=0.58; IR(neat, cm⁻¹): 3360, 2980, 1744, 1200, 1170; ¹H NMR (500 MHz, CDCl₃): δ 6.95 (d, J=2.7 Hz, 1H), 6.71 (m, 1H), 6.66 (m, 1H), 6.52 (dd, J=1.7, 15.8 Hz, 1H), 6.17 (dq, J=6.6, 11.2 Hz, 1H), 1.88 (dd, J=1.7, 6.6 Hz, 3H), 1.34 (s, 9H); ¹³C NMR (100 MHz, CDCl₃): δ 177.9, 150, 144.4, 128.5, 126, 124.8, 120.3, 119.4, 116.3, 39, 27.1, 18.7; LRMS (EI+): 234 (M+), 150 (M+−pivoyl); HRMS(EI+): calculated for C₁₄H₁₈O₃, 234.1256; found 234.1261.

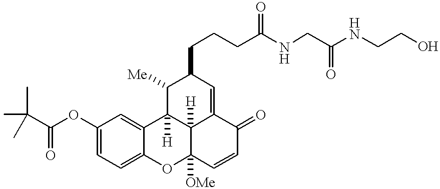

PIV Hetero

To a 10 mL BioRad tube was placed PS-DES (x) (300 mg, 0.057 mmol, 0.19 mmol/g) and the 4-pivoyl-2-[E-1-propenyl]phenol (X) (189 mg, 0.85 mmol, 15 equiv.). CH₂Cl₂ (3 mL) and THF (1 mL) were added to swell the resin and dissolve the pivolate. Then, the BioRad tube was placed on an orbital stirrer and was allowed to stir for 30 minutes to afford good mixing. After this time, the IPh(OAc)₂ (275 mg, 0.85 mmol, 15 equiv.) was added, the tube was shaken vigorously, then placed on an orbital stirrer and agitated 2 hours. During this time, the resin/solution darkened to a deep orange. Then, the tube was attached to a Promega wash station, and the resin was washed (×8): CH₂Cl₂, 1% Et₃N/CH₂Cl₂, THF, MeOH, H₂O, CH₃CN and then dried. The resin was then transferred into another 10 mL BioRad tube, swollen with 1.5 mL of THF, and HF•pyridine (100 μL) added. Again, the tube was placed on an orbital stirrer and was allowed to stir for 2 hours. Then, TMSOMe (0.5 mL) was added and again, the resin was allowed to stir for 2 hours. After this time, the resin was filtered and washed CH₂Cl₂ to afford a yellow-orange foam upon concentration. Column chromatography [9:1/CH₂Cl₂:MeOH] afforded 26 mg (79%) of a colorless film. TLC [9:1/CH₂Cl₂:MeOH] R_{f}=0.13; IR(neat, cm⁻¹): 3440, 2980, 2790, 1740, 1680, 1630, 1610, 1140; ¹H NMR (500 MHz, CDCl₃): δ 7.24 (d, J=10.3 Hz, 1H), 7.07 (m, 1H), 7.03 (d, J=2.2 Hz, 1H), 6.82 (m, 1H), 6.79 (t, J=5.7 Hz, 1H), 6.75 (m, 1H), 6.6 (t, J=5.7 Hz, 1H), 6.33 (d, J=10.3 Hz, 1H), 3.78 (abquart., J=5.8, 20.3 Hz, 2H), 3.65 (m, 2H), 3.38 (m, 1H), 3.33 (m, 1H), 3.29 (s, 3H), 3.16 (dt, J=2.4, 7.2 Hz, 1H), 2.64 (m, 1H), 2.11 (m, 2H), 2.00 (m, 2H), 1.6 (m, 2H), 1.3 (s, 9H), 1.13 (d, J=7.1 Hz, 3H), 0.87 (m, 2H); ¹³C NMR (100 MHz, CDCl₃): δ 186.5, 178.6, 174.4, 170.2, 148.5, 145.2, 142.1, 131.8, 128.1, 125.9, 121.1, 120.3, 118.9, 96, 61.9, 49.3, 43.8, 42.3, 42.1, 37, 36.4, 35.4, 33.8, 33.5, 27.1, 25, 21.6; LRMS (TOF, ES+): 591 (M+Na), 569 (M+); HRMS(TOF, ES+): calculated for C₃₁H₄₀O₈N₂, 591.2682 (M+Na); found 591.2709.

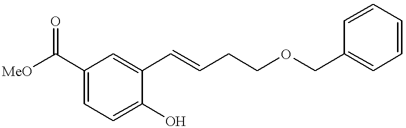

4-Carbomethoxy2-[4-benzyloxy-E-1-butenyl]phenol (X)

To an oven-dried 50 mL flask, equipped with stir bar and double septaed was cooled/purged under a stream of Ar(g), and was then charged with 4-carbomethoxy-2-hydroxybenzaldehyde (840 mg, 4.7 mmol), dry THF (20 mL) and colled to 0° C. In another oven-dried 200 mL flask, equipped with stir bar and double septaed was cooled/purged under a stream of Ar(g), and was then charged with 3-benzyloxypropyl triphenylphosphonium bromide (2.55 g, 5.18 mmol), dry THF (50 mL) and cooled to 0° C. Then, one equivalent of freshly prepared LiN(TMS)₂ (4.7 mL, 4.7 mmol, 1.0 M THF) was added via syringe and was allowed to stir at 0° C. for 30 minutes to form the phenoxide. Then, 1.1 equivalents of freshly prepared LiN(TMS)₂ (5.2 mL, 5.2 mmol, 1.0 M THF) was added via syringe and was allowed to stir at 0° C. for 30 minutes to form the ylide. After 30 minutes, the phenoxide was transferred via cannula to the ylide, and was allowed to slowly warm to room temperature over 3 hours. The reaction was quenched with 0.5 M HCl, extracted into EtOAc, washed with water, brine, and dried over anhydrous Na₂SO₄. Concentration in vactio and column chromatography [80:20-50:50/Hex:EtOAc] afforded 1.19 g (82%) of a white solid. TLC [50:50/Hex:EtOAc] R_{f}=0.58; IR (neat, cm⁻¹): 3325, 3010, 2970, 1715 (s), 1685, 1602, 1276, 1121; ¹H NMR (500 MHz, CDCl₃): δ 8.01 (d, J=2.04 Hz, 1H), 7.76 (dd, J=2.0, 8.4 Hz, 1H), 7.3 (m, 4H), 7.29 (m, 1H), 6.78 (d, J=8.4 Hz, 1H), 6.65 (d, J=16 Hz, 1H), 6.5 (s, 1H), 6.26 (dt, J=6.9, 16 Hz, 1H), 4.56 (s, 2H), 3.88 (s, 3H), 3.63 (t, J=6.5 Hz, 2H), 2.57 (q, J=6.5 Hz, 2H), ¹³C NMR (100 MHz, CDCl₃): δ 167.3, 157.2, 137.8, 129.8, 129.1, 128.3, 127.8, 127.7, 125.4, 124.7, 122, 115.5, 72.9, 69.5, 51.9, 33.7; LRMS (CI+): 330 (M+NH₄), 238 (M+NH₄−Bn), 170; HRMS (CI+): calculated for C₁₉H₂₀O₄, 330.1706 (M+NH₄); found 330.1699.

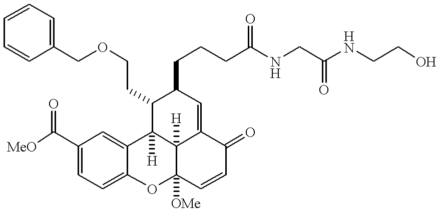

Ester Heterodimer (X)

To a 10 mL BioRad tube was placed PS-DES (x) (300 mg, 0.057 mmol, 0.19 mmol/g) and the 4-carbomethoxy-2-[4-benzyloxy-E-1-butenyl]phenol (X) (266 mg, 0.85 mmol, 15 equiv.). CH₂Cl₂ (3 mL) and THF (1 mL) were added to swell the resin and dissolve the ester. Then, the BioRad tube was placed on an orbital stirrer and was allowed to stir for 30 minutes to afford good mixing. After this time, the IPh(OAc)₂ (275 mg, 0.85 mmol, 15 equiv.) was added, the tube was shaken vigorously, then placed on an orbital stirrer and agitated 2 hours. During this time, the resin/solution darkened to a deep orange. Then, the tube was attached to a Promega wash station, and the resin was washed (×8): CH₂Cl₂, 1% Et₃N/ CH₂Cl₂, THF, MeOH, H₂O, CH₃CN and then dried. The resin was then transferred into another 10 mL BioRad tube, swollen with 1.5 mL of THF, and HF•pyridine (100 μL) added. Again, the tube was placed on an orbital stirrer and was allowed to stir for 2 hours. Then, TMSOMe (0.5 mL) was added and again, the resin was allowed to stir for 2 hours. After this time, the resin was filtered and washed CH₂Cl₂ to afford a yellow-orange foam upon concentration. Column chromatography [9:1/CH₂Cl₂:MeOH] afforded 30 mg (81%) of a colorless film. TLC [9:1/CH₂Cl₂:MeOH] R_{f}=0.18; IR(neat, cm⁻¹): 3380, 2980, 2760, 1714, 1680, 1636, 1276; ¹H NMR (500 MHz, CDCl₃): δ 8.19 (s, 1H), 7.77 (d, J=19.1 Hz, 1H), 7.36 (m, 5H), 7.25 (d, J=10.4 Hz, 1H), 6.98 (m, 1H), 6.85 (d, J=8.5 Hz, 1H), 6.73 (t, J=5.7 Hz, 1H), 6.52 (t, J=5.7 Hz, 1H), 6.33 (d, J=10.2 Hz, 1H), 4.55 (s, 2H), 3.87 (abquart., J=6.2, 41 Hz, 2H), 3.95 (s, 3H), 3.63 (m, 4H), 3.48 (d, J=6.6 Hz, 1H), 3.36 (t, J=5.2 Hz, 1H), 3.32 (s, 3H), 3.16 (m, 1H), 2.95 (m, 1H), 2.13 (m, 1H), 1.95 (m, 1H), 1.64 (m, 4H), 1.00 (m, 1H), 0.45 (m, 1H); ¹³C NMR (100 MHz, CDCl₃): δ 186, 173.5, 167.7, 155.3, 142.5, 141.6, 137.9, 132, 130.4, 129.1, 128.6, 127.9, 124.6, 123.3, 117.5, 96.1, 73.3, 68.1, 61.6, 52.3, 49.5, 43.5, 42, 39.9, 37, 36.6, 34.6, 33.8, 32.6, 31.6, 24.8; LRMS (TOF, ES+): 647 (M+1), 646 (M+) 615 (M+−OMe); HRMS(TOF, ES+): calculated for C₃₆H₄₂O₉N₂, 647.2968; found 647.2954.

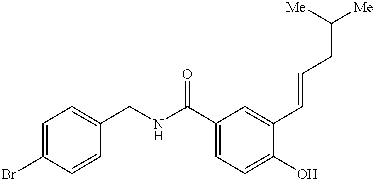

4-(para-Bromocarboxamide)-2-[E-1-(4-methyl)butenyl]phenol (X)

To an oven-dried 200 mL flask, equipped with stir bar and double septaed was cooled/purged under a stream of Ar(g), and was then charged with 4-carboxy-2-[E-1-(4-methyl)butenyl]phenol (1.7 g, 7.7 mmol), PyBOP (7 g, 13.5 mmol), and 4-bromobenzylamine (2.6 g, 11.5 mmol). Then, dry DMF (25 mL) and CH₂Cl₂ (25 mL) were added followed by cooling to 0° C. Next, diisopropylethylarnine (4.0 mL, 23.2 mmol) was added, and the reaction was allowed to slowly warm to room temperature overnight. The reaction was quenched with 0.5 M HCl, extracted into EtOAc, washed with water, brine, and dried over anhydrous Na₂SO₄. Concentration in vacuo and column chromatography [50:50/Hex:EtOAc] afforded 2.4 g (80%) of a pale yellow solid. TLC [50:50/Hex:EtOAc] R_{f}=0.24; IR (neat, cm⁻¹): 3360, 2980, 1700, 1630, 1537, 624; ¹H NMR (500 MHz, DMSO-d₆): δ 10.09 (s, 1H), 8.85 (t, J=5.4 Hz, 1H), 7.97 (s, 1H), 7.61 (d, J=8.2 Hz, 1H), 7.49 (d, J=8.1 Hz, 2H), 7.25 (d, J=8.1 Hz, 2H), 6.85 (d, J=8.4 Hz, 1H), 6.59 (d, J=16 Hz, 1H), 6.28 (m, 1H), 4.41 (d, J=5.6 Hz, 2H), 2.07 (t, J=6.6 Hz, 2H), 1.69 (hept., J=6.6 Hz, 1H), 0.91 (d, J=6.6 Hz, 6H); ¹³C NMR (100 MHz, DMSO-d₆): δ 165.9, 156.8, 131, 129.5, 129.3, 127.4, 125.3, 125.1, 124.9, 123.7, 119.6, 115.1, 42.2, 41.9, 28, 22.2; LRMS (TOF, ES+): 390 (M+⁸¹Br), 388 (M+⁷⁹Br), 371 (M+−OH); HRMS (TOF, ES+): calculated for C₂₀H₂₂O₂NBr, 388.0912; found 388.0923.

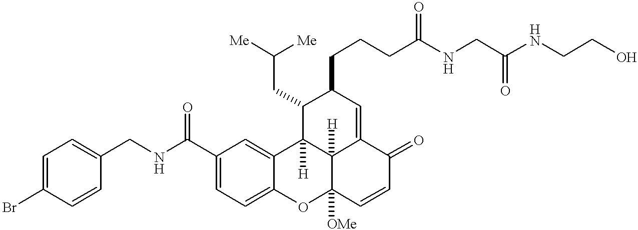

Heterodimer (X)

To a 10 mL BioRad tube was placed PS-DES (x) (300 mg, 0.057 mmol, 0.19 mmol/g) and the 4-(para-bromocarboxamide)-2-[E-1-(4-methyl)butenyl]phenol (X) (221 mg, 0.57 mmol, 10 equiv.). CH₂Cl₂ (3 mL) and THF (1 mL) were added to swell the resin and dissolve the amide. Then, the BioRad tube was placed on an orbital stirrer and was allowed to stir for 30 minutes to afford good mixing. After this time, the IPh(OAc)₂ (183 mg, 0.57 mmol, 10 equiv.) was added, the tube was shaken vigorously, then placed on an orbital stirrer and agitated 2 hours. During this time, the resin/solution darkened to a deep orange. Then, the tube was attached to a Promega wash station, and the resin was washed (×8): CH₂Cl₂, 1% Et₃N/CH₂Cl₂, THF, MeOH, H₂O, CH₃CN and then dried. The resin was then transferred into another 10 mL BioRad tube, swollen with 1.5 mL of THF, and HF•pyridine (100 μL) added. Again, the tube was placed on an orbital stirrer and was allowed to stir for 2 hours. Then, TMSOMe (0.5 mL) was added and again, the resin was allowed to stir for 2 hours. After this time, the resin was filtered and washed CH₂Cl₂ to afford a yellow-orange foam upon concentration. Column chromatography [9:1/CH₂Cl₂:MeOH] afforded 33 mg (78%) of a colorless film. TLC [9:1/CH₂Cl₂:MeOH] R_{f}=0.21; IR (neat, cm⁻¹): 3370, 2980, 1700, 1680, 1535, 600; ¹H NMR (500 MHz, CDCl₃): δ 7.98 (s, 1H), 7.46 (d, J=8.4 Hz, 1H), 7.41 (dd, J=1.6, 8.4 Hz, 2H), 7.23 (m, 2H), 7.04 (t, J=2.1 Hz, 1H), 6.97 (m, 1H), 6.84 (d, J=8.4 Hz, 1H), 6.78 (m, 2H), 6.33 (d, J=10.3 Hz, 1H), 4.56 (abquart., J=5.9, 29 Hz, 2H), 3.66 (t, J=4.4 Hz, 2H), 3.4 (m, 1H), 3.32 (s, 3H), 3.15 (dt, J=2.5, 7 Hz, 1H), 2.7 (m, 1H), 2.06 (m, 4H), 1.78 (m, 4H), 1.59 (m, 1H), 1.27 (m, 2H), 1.07 (m, 1H), 0.98 (dd, J=1.6, 5 Hz, 6H), 0.346 (m, 1H); ¹³C NMR (125 MHz, CDCl₃): δ 186.1, 174., 170.2, 168.1, 154.2, 143, 141.5, 137, 132, 131.9, 129.3, 129.1, 128.3, 127.9, 126.2, 125.5, 121.5, 117.4, 96.1, 61.9, 49.4, 44.1, 43.6, 43.5, 42.4, 40.5, 37, 36.9, 33.7, 32.9, 31.9, 29.7, 25.5, 25.1, 22.8; LRMS (FAB+): 746 (M+Na, ⁸¹Br), 744 (M+Na, ⁷⁹Br), 604, 329; HRMS(FAB+): calculated for C₃₇H₄₄O₇N₃Br(Na), 744.2248; found 744.2260.

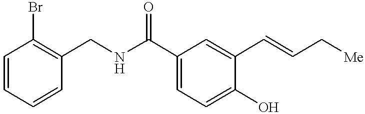

4-(ortho-Bromobenzylcarboxamide)-2-[E-1-butenyl]phenol (X)

To an oven-dried 50 mL flask, equipped with stir bar and double septaed was cooled/purged under a stream of Ar(g), and was then charged with 4-carboxy-2-[E-1-butenyl]phenol (288 mg, 1.5 mmol), PyBOP (1.17 g, 2.25 mmol), and 2-bromobenzylamine (417 mg, 2.25 mmol). Then, dry DMF (10 mL) was added followed by cooling to 0° C. Next, diisopropylethylamine (0.80 mL, 4.5 mmol) was added, and the reaction was allowed to slowly warm to room temperature overnight. The reaction was quenched with 0.5 M HCl, extracted into EtOAc, washed with water, brine, and dried over anhydrous Na₂SO₄. Concentration in vacuo and column chromatography [50:50/Hex:EtOAc] afforded 452 mg (84%) of a white foam. TLC [50:50Hex:EtOAc] R_{f}=0.26; IR (neat, cm⁻¹): 3410, 2980, 1702, 1630, 1537, 610; ¹H NMR (500 MHz, CDCl₃): δ 7.77 (d, J=2.2 Hz, 1H), 7.57 (dd, J=1.1, 7.9 Hz, 1H), 7.48 (dd, J=2.2, 8.4 Hz, 1H), 7.46 (dd, J=1.6, 7.6 Hz, 1H), 7.28 (td, J=1.1, 7.4 Hz, 1H), 7.15 (td, J=1.6, 7.7 Hz, 1H), 6.82 (d, J=8.4 Hz, 1H), 6.64 (s, 1H), 6.6 (t, J=5.7 Hz, 1H), 6.56 (dt, J=1.4, 16 Hz, 1H), 6.28 (dt, 6.5, 16 Hz, 1H), 4.7 (d, J=6 Hz, 2H), 2.24 (quint., J=7.7 Hz, 2H), 1.08 (t, J=7.7 Hz, 3H); ¹³C NMR (100 MHz, CDCl₃): δ 167.9, 156.3, 137, 135.2, 132.8, 130.4, 129.2, 127.7, 126.7, 126.1, 125.3, 123.7, 122.5, 115.8, 44.4, 26.4, 13.5; LRMS (TOF, ES+): 362 (M+⁸¹Br), 360 (M+⁷⁹Br); HRMS (TOF, ES+): calculated for C₁₈H₁₈O₂NBr, 360.0599; found 360.0584.

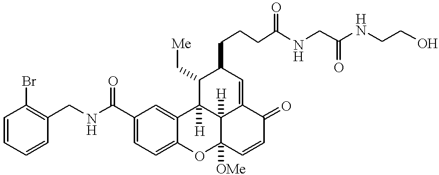

Heterodimer (X). Two Routes: A) Direct Heterodimerization

To a 10 mL BioRad tube was placed PS-DES (x) (300 mg, 0.057 mmol, 0.19 mmol/g) and the 4-(ortho-bromocarboxamide)-2 -[E-1-butenyl]phenol (X) (200 mg, 0.57 mmol, 10 equiv.). CH₂Cl₂ (3 mL) and THP (1 mL) were added to swell the resin and dissolve the amide. Then, the BioRad tube was placed on an orbital stirrer and was allowed to stir for 30 minutes to afford good mixing. After this time, the IPh(OAc)₂ (183 mg, 0.57 mmol, 10 equiv.) was added, the tube was shaken vigorously, then placed on an orbital stirrer and agitated 2 hours. During this time, the resin/solution darkened to a deep orange. Then, the tube was attached to a Promega wash station, and the resin was washed (×8): CH₂Cl₂, 1% Et₃N/CH₂Cl₂, THF, MeOH, H₂O, CH₃CN and then dried. The resin was then transferred into another 10 mL BioRad tube, swollen with 1.5 mL of THF, and HF•pyridine (100 μL) added. Again, the tube was placed on an orbital stirrer and was allowed to stir for 2 hours. Then, TMSOMe (0.5 mL) was added and again, the resin was allowed to stir for 2 hours. After this time, the resin was filtered and washed CH₂Cl₂ to afford a yellow-orange foam upon concentration. Column chromatography [9:1/CH₂Cl₂:MeOH] afforded 31 mg (77%) of a colorless film. TLC [9:1/CH₂Cl₂:MeOH] R_{f}=0.22; B)

Displacement of Solid Phase Activated Ester

To a 10 mL BioRad tube was placed PS-DES (x) (300 mg, 0.057 mmol, 0.19 mmol/g) and the 4-(N-hydroxysuccinimide ester)-2-[E-1-butenyl]phenol (X) (247 mg, 0.85 mmol, 15 equiv.). CH₂Cl₂ (3 mL) and THF (1 mL) were added to swell the resin and dissolve the activated ester. Then, the BioRad tube was placed on an orbital stirrer and was allowed to stir for 30 minutes to afford good mixing. After this time, the IPh(OAc)₂ (275 mg, 0.85 mmol, 15 equiv.) was added, the tube was shaken vigorously, then placed on an orbital stirrer and agitated 2 hours. During this time, the resin/solution darkened to a deep orange. Then, the tube was attached to a Promega wash station, and the resin was washed (×8): CH₂Cl₂, 1% Et₃N/CH₂Cl₂, THF, MeOH, H₂O, CH₃CN and then dried. The resin was then transferred into another 10 mL BioRad tube, swollen with CH₂Cl₂ (3 mL) followed by ortho-bromobenzylamine (127 mg, 0.57 mmol, 10 equiv.) and 2,6-lutidine (66 μL, 0.57 mmol, 10 equiv.). Then, the tube was shaken vigorously and placed on an orbital stirrer and agitated for 8 hours. Then, the tube was attached to a Promega wash station, and the resin was washed (×8): CH₂Cl₂, THF, MeOH, H₂O, CH₃CN and then dried. The resin was then transferred into another 10 mL BioRad tube, swollen with 1.5 mL of THF, and HF•pyridine (100 μL) added. Again, the tube was placed on an orbital stirrer and was allowed to stir for 2 hours. Then, TMSOMe (0.5 mL) was added and again, the resin was allowed to stir for 2 hours. After this time, the resin was filtered and washed CH₂Cl₂ to afford a yellow-orange foam upon concentration. Column chromatography [9:1/CH₂Cl₂:MeOH] afforded 25.3 mg (64%) of a colorless film. TLC [9:1/CH₂Cl₂:MeOH] R_{f}=0.21; IR (neat, cm⁻¹): 3390, 2978, 1701, 1683, 1536, 598; ¹H NMR (500 MHz, CDCl₃): δ 8.00 (s, 1H), 7.58 (d, J=7.9 Hz, 1H), 7.41 (m, 2H), 7.31 (t, J=7.5 Hz, 1H), 7.16 (m, 3H), 6.94 (m, 1H), 6.85 (m, 3H), 6.33 (d, J=10.3 Hz, 1H), 4.67 (m, 2H), 3.88 (abquart, J=5.7, 41 Hz, 2H), 3.69 (t, J=5.4 Hz, 2H), 3.49 (m, 1H), 3.33 (s, 31), 3.15 (m, 1H), 2.55 (m, 1H), 2.11 (m, 1H), 1.92 (m, 2H), 1.74 (m, 4H), 1.59 (m, 2H), 1.49 (m, 1H), 1.35 (m, 1H), 1.04 (t, J=7.2 Hz, 3H), 0.92 (m, 1H), 0.29 (m, 1H); ¹³C NMR (125 MHz, CDCl₃): δ 186.1, 174.4, 170.3, 154.3, 143, 141.5, 136.8, 133, 132, 130.3, 129.5, 128.4, 127.8, 127.2, 125.5, 125.1, 123.7, 117.4, 96.1, 62, 49.4, 44.6, 43.8, 39.8, 37.2, 36.8, 33.5, 31.8, 27.7, 25.1, 12.4; LRMS (FAB+): 718 (M+Na, ⁸¹Br), 716 (M+Na, ⁷⁹Br), 358, 301; HRMS (FAB+): calculated for C₃₅H₄₀O₇N₃Br(Na), 716.1947; found 716.1933.

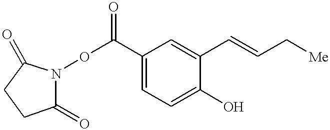

4-(N-Hydroxysuccinimide ester)-2-[E-1-butenyl]phenol (X)

To an oven-dried 50 mL flask, equipped with stir bar and double septaed was cooled/purged under a stream of Ar(g), and was then charged with 4-carbomethoxy-2-[E-1-butenyl]phenol (1.76 g, 9.16 mmol), EDC (3.5 g, 18.3 mmol), and N-hydroxysuccinimide (1.58 g, 13.75 mmol). Then, dry DMF (20 mL)/CH₂Cl₂ (20 mL) were added followed by cooling to 0° C. Next, diisopropylethylamine (4.8 mL, 27.5 mmol) was added, and the reaction was allowed to slowly warm to room temperature overnight. The reaction was quenched with 0.5 M HCl, extracted into EtOAc, washed with water, brine, and dried over anhydrous Na₂SO₄. Concentration in vacuo and column chromatography [50:50/Hex:EtOAc] afforded 1.82 g (70%) of a white foam. TLC [50:50/Hex:EtOAc] R_{f}=0.17; IR (neat, cm⁻¹): 3430, 2980, 1720, 1688, 1540, 1230, 1110; ¹H NMR (500 MHz, CDCl₃): δ 8.0 (d, J=2.1 Hz, 1H), 7.72 (dd, J=2.1, 8.4 Hz, 1H), 6.78 (d, J=8.4 Hz, 1H), 6.47 (d, J=16 Hz, 1H), 6.37 (s, 1H), 6.26 (dt, J=6.4, 16 Hz, 1H), 2.91 (bs, 4H), 2.25 (quint., J=7.6 Hz, 2H), 1.09 (t, J=7.4 Hz, 3H); ¹³C NMR (100 MHz, CDCl₃): δ 170.1, 161.4, 158.6, 136.2, 130.6, 129.9, 125.7, 121.6, 116.4, 115.9, 26.3, 25.6, 13.4; LRMS (FAB+): 312 (M+Na), 289 (M+); HRMS (FAB+): calculated for C₁₅H₁₅NO₅(Na), 312.0848; found 312.0853.

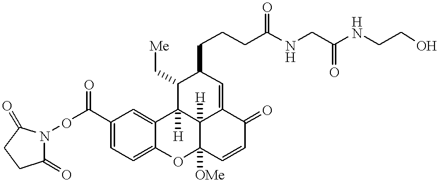

Act. Ester Heterodimer (X)

To a 10 mL BioRad tube was placed PS-DES (x) (300 mg, 0.057 mmol, 0.19 mmol/g) and the 4-(N-Hydroxysuccinimide ester)-2-[E-1-butenyl]phenol (X) (247 mg, 0.85 mmol, 15 equiv.). CH₂Cl₂ (3 mL) and THF (1 mL) were added to swell the resin and dissolve the amide. Then, the BioRad tube was placed on an orbital stirrer and was allowed to stir for 30 minutes to afford good mixing. After this time, the IPh(OAc)₂ (275 mg, 0.85 mmol, 15 equiv.) was added, the tube was shaken vigorously, then placed on an orbital stirrer and agitated 2 hours. During this time, the resin/solution darkened to a deep orange. Then, the tube was attached to a Promega wash station, and the resin was washed (×8): CH₂Cl₂, 1% Et₃N/CH₂Cl₂, THF, MeOH, H₂O, CH₃CN and then dried. The resin was then transferred into another 10 mL BioRad tube, swollen with 1.5 mL of THF, and HF•pyridine (100 μL) added. Again, the tube was placed on an orbital stirrer and was allowed to stir for 2 hours. Then, TMSOMe (0.5 mL) was added and again, the resin was allowed to stir for 2 hours. After this time, the resin was filtered and washed CH₂Cl₂ to afford a yellow-orange foam upon concentration. Column chromatography [9:1/CH₂Cl₂:MeOH] afforded 26 mg (73%) of a colorless film. TLC [9:1/CH₂Cl₂:MeOH] R_{f}=0.21; IR (neat, cm⁻¹):3360, 2980, 1720, 1685, 1545, 1230, 1110; (mixture of rotamers) ¹H NMR (500 MHz, CDCl₃): δ 8.19 (s, 1H), 7.92 (d, J=7.6 Hz, 1H), 7.25 (m, 1H), 6.95 (m, 1H), 6.93 (d, J=8.5 Hz, 1H), 6.72 (t, J=5.7 Hz, 1H), 6.58 (t, J=5.7 Hz, 1H), 6.34 (d, J=10.3 Hz, 1H), 3.8 (abquart., J=6.3, 41 Hz, 2H), 3.6 (m, 2H), 3.52 (m, 1H), 3.4 (m, 1H), 3.35 (s, 3H), 3.17 (m, 1H), 2.95 (bs, 4H), 2.49 (m, 1H), 2.15 (m, 1H), 2.03 (m, 1H), 1.84 (m, 1H), 1.69 (m, 3H), 1.52 (m, 1H), 1.50 (m, 1H), 1.38 (m, 1H), 1.06 (t, J=7.4 Hz, 3H), 0.93 (m, 1H), 0.345 (m, 1H); ¹³C NMR (100 MHz, CDCl₃): δ 185.5, 173.7, 171.9, 170.9, 165.8, 151.6, 143, 141.1, 132.2, 131.6, 130.1, 127.5, 125.5, 118.2, 95.8, 62, 49.6, 43.8, 42.4, 40.1, 37.8, 36.8, 36.2, 33.6, 31.5, 27.6, 25.8, 25.6, 24.3, 12.4; LRMS (FAB+): 646 (M+Na), 624 (M+), 460, 307; HRMS (FAB+): calculated for C₃₂H₃₇O₁₀N₃(Na), 646.2377; found 646.2381.

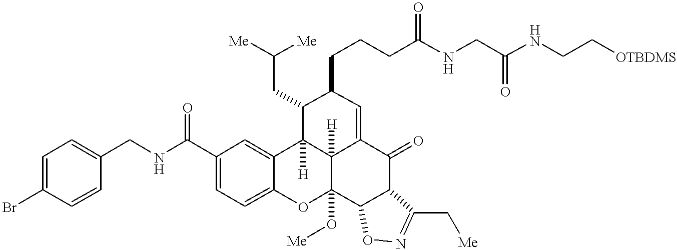

Hetero-nitrile oxide Cycloaddition (X)

To a 20 mL BioRad tube was placed PS-DES (X) (500 mg, 0.1 mmol, 0.20 mmol/g) and the 4-(para-bromocarboxamide)-2-[E-I-(4-methyl)butenyl]phenol (X) (388 mg, 1.0 mmol, 10 equiv.). CH₂Cl₂ (8 mL) and THF (2 mL) were added to swell the resin and dissolve the amide. Then, the BioRad tube was placed on an orbital stirrer and was allowed to stir for 30 minutes to afford good mixing. After this time, the IPh(OAc)₂ (322 mg, 1.0 mmol, 10 equiv.) was added, the tube was shaken vigorously, then placed on an orbital stirrer and agitated 2 hours. During this time, the resin/solution darkened to a deep orange. Then, the tube was attached to a Promega wash station, and the resin was wasted (×8): CH₂Cl₂, 1% Et₃N/CH₂Cl₂, THF, MeOH, H₂O, CH₃CN and then dried. Then, the resin was placed into a 20 mL PEG bottle with stir bar, swollen with CH₂Cl₂ (10 mL) and a catalytic amount of Et₃N (25 μL) was added. The PEG bottle was the cooled to 0° C., and nitropropane (89 μL, 1.0 mmol, 10 equiv.) and PhNCO (457 μL, 4.2 mmol, 42 equiv.) were added via syringe. The reaction was allowed to go at 0° C. for 8 hours. After this time, the resin transfered to a 20 mL BioRad tube and was attached to a Promega wash station, and the resin was washed (×8): CH₂Cl₂, THF, MeOH, H₂O, CH₃CN and then dried. The resin was then transferred into another 20 mL BioRad tube, swollen with 6 mL of THF, and HF•pyridine (500 μL) added. Again, the tube was placed on an orbital stirrer and was allowed to stir for 2 hours. Then, TMSOMe (1.5 mL) was added and again, the resin was allowed to stir for 2 hours. After this time, the resin was filtered and washed CH₂Cl₂ to afford a yellow foam upon concentration. The crude cycloaddition product was placed in an oven-dried flask, equipped with stir bar, and charged with TBDMSCl (45 mg, 0.3 mmol) and imidazole (20 mg, 0.3 mmol). Then, dry CH₂Cl₂ (3 mL) was added, and the reaction was allowed to stir overnight at room temperature. . The reaction was quenched water, extracted into EtOAc, washed with water, brine, and dried over anhydrous Na₂SO₄. Concentration in vacuo and column chromatography [9:1/ EtOAc:hexanes] afforded 56.2 mg (62%) of an 18:1 mixture of cycloadducts as a colorless film. TLC [9:1/EtOAc:hexanes] R_{f}=0.12; IR (neat, cm⁻¹): 3307, 1643, 1539, 1487, 1250, 1106; ¹H NMR (400 MHz, CDCl₃): δ 7.92 (s, 1H), 7.45 (d, J=8.3 Hz, 2H), 7.4 (d, J=8.6 Hz, 1H), 7.21 (d, J=8.3 Hz, 2H), 7.04 (t, J=5 Hz, 1H), 6.94 (m, 1H), 6.82 (m, 1H), 6.3 (t, J=4.5 Hz, 1H), 5.22 (d, J=11.2 Hz, 1H), 4.57 (abquart., J=6.1, 36.9 Hz, 2H), 4.2 (d, J=11.2 Hz, 1H), 3.78 (abquart., J=5.6, 42.3 Hz, 2H), 3.4 (m, 1H), 3.34 (s, 3H), 3.15 (m, 1H), 2.7 (m, 1H), 2.45 (m, 1H), 2.29 (m, 1H), 1.98 (m, 3H), 1.85 (m, 1H), 1.71 (m, 2H), 1.3 (m, 2H), 1.21 (t, J=7.4 Hz, 3H), 1.12 (m, 1H), 0.91 (m, 1H), 0.87 (s, 9H), 0.56 (m, 1H), 0.051 (s, 6H); ¹³C NMR (100 MHz, CDCl₃): δ 188.7, 173.4, 168.9, 167.6, 158.1, 153.3, 147.1, 137.4, 131.8, 129.4, 128.5, 127.9, 127.7, 125.8, 125.4, 122.2, 117.7, 97, 61.6, 60.5, 49.5, 44.7, 43.5, 41.6, 40.8, 36.8, 34.3, 32.5, 32.1, 31.4, 25.8, 25.2, 24.9, 22.8, 22.7, 20, 18.2, 10.7, −5.3; LRMS (TOF, ES+): 910 (M+1 ⁸¹Br), 909 (M+⁸¹Br), 908 (M+1 ⁷⁹Br), 907 (M+ ⁷⁹Br); HRMS (TOF, ES+): calculated for C₄₆H₆₃N₄O₈SiBr, 907.3647; found 907.3636.

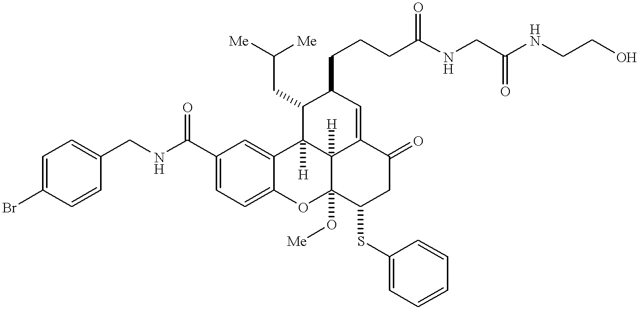

Hetero-Thiphenol Conjugate Addition (X)

To a 20 mL BioRad tube was placed PS-DES (X) (500 mg, 0.1 mmol, 0.20 mmol) and the 4-(para-bromocarboxamide)-2-[E-1-(4-methyl)butenyl]phenol (X) (388 mg, 1.0 mmol, 10 equiv.). CH₂Cl₂ (8 mL) and THF (2 mL) were added to swell the resin and dissolve the amide. Then, the BioRad tube was placed on an orbital stirrer and was allowed to stir for 30 minutes to afford good mixing. After this time, the IPh(OAc)₂ (322 mg, 1.0 mmol, 10 equiv.) was added, the tube was shaken vigorously, then placed on an orbital stirrer and agitated 2 hours. During this time, the resin/solution darkened to a deep orange. Then, the tube was attached to a Promega wash station, and the resin was washed (×8): CH₂Cl₂, 1% Et₃N/CH₂Cl₂, THF, MeOH, H₂O, CH₃CN and then dried. Then, the resin was placed into another 20 mL BioRad tube, swollen with THF (8 mL), thiophenol (31 μL, 0.3 mmol, 3.0 equiv.) was added, followed by a catalytic amount of Et₃N(5 μL). The tube was shaken, and then placed on an orbital stirrer and agitated for 24 hours. After this time, the resin was again attached to a Promega wash station, and the resin was washed (×8): CH₂Cl₂, 1% Et₃N/CH₂Cl₂, THF, MeOH, H₂O, CH₃CN and dried. The dried resin was then placed in another BioRad tube, swollen with THF (6 mL) and HF•pyridine (500 μL) was added. Again, the tube was placed on an orbital stirrer and was allowed to stir for 2 hours. Then, TMSOMe (1.5 mL) was added and again, the resin was allowed to stir for 2 hours. After this time, the resin was filtered and washed CH₂Cl₂ to afford a yellow foam upon concentration. Column chromatography [9:1/CH₂Cl₂:MeOH] afforded 58.5 mg (70%) of the a single diastereomer as a colorless film. TLC [9:1/CH₂Cl₂:MeOH] R_{f}=0.12; IR (neat, cm⁻¹): 3306, 1641, 1537, 1487, 1140, 754; ¹H NMR (500 MHz, CDCl₃): δ 7.97 (s, 1H), 7.50 (dd, J=2.1, 7.6 Hz, 2H), 7.45 (d, J=8.3 Hz, 1H), 7.43 (d, J=4.9 Hz, 1H), 7.35 (m, 3H), 7.2 (d, J=8.3 Hz, 2H), 7.09 (t, J=5.5 Hz, 1H), 6.83 (m, 3H), 6.67 (t, J=3 Hz, 1H), 4.56 (abquart., J=6.1, 27.4 Hz, 2H), 4.20 (dd, J=1.8, 5.3 Hz, 1H), 3.81 (abquart., J=5.8, 21 Hz, 2H), 3.64 (t, J=5.1 Hz, 2H), 3.36 (m, 3H), 3.31 (s, 3H), 2.99 (dd, J=5.4, 17.8 Hz, 1H), 2.74 (m, 2H), 2.0 (m, 3H), 1.85 (m, 3H), 1.72 (m, 3H), 1.6 (m, 3H), 1.31 (m, 3H), 1.0 (dd, J=6.4, 13.5 Hz, 6H), 0.86 (m, 1H), 0.48 (m, 1H); ¹³C NMR (125 MHz, CDCl₃): δ 196.8, 174, 170.1, 167.9, 154.1, 142.9, 137.1, 134, 131.87, 131.81, 130.3, 129.4, 129.2, 128.5, 128.4, 126.9, 125.5, 125.3, 121.4, 117.4, 99.5, 61.8, 48.5, 46.6, 44.3, 43.59, 43.54, 42.4, 41.4, 40.2, 36.9, 34.1, 33.7, 33.2, 31.8, 29.6, 25.3, 25.1, 23, 22.7; LRMS (TOF, ES+): 835 (M+1 ⁸¹Br), 834 (M+ ⁸¹Br), 833 (M+1 ⁷⁹Br), 832 (M+ ⁷⁹Br); HRMS (TOF, ES+): calculated for C₄₃H₅₀N₃O₇SBr, 832.2631; found 832.2628.

Supporting Information

Experimental Section

General Procedures. All reactions were performed in flamed-dried glassware under a positive pressure of argon. Flash column chromatography was performed as described by Still et al. (Still, W. C.; Kahn, M.; Mitra, A. J. Org. Chem. 1978, 43, 2923.) employing E. Merck silica gel 60 (230-400 mesh ASTM).

Materials. Tetrahydrofuran and ether were distilled under nitrogen from sodium-benzophenone ketyl. Toluene was distilled under nitrogen from calcium hydride. Chloroform, pentane, n-hexane, benzene, and pyridine were distilled under argon from calcium hydride. Lithium chloride was dried under vacuum at 45° C. overnight prior to use. Molecular sieves (4 Å, powder, <5 micron) were flame dried under vacuum prior to use. The molarity of Grignard reagents was determined by quenching with water and titrating with 0.1N aqueous hydrochloric acid solution against phenol red indicator.

Instrumentation. Infrared spectra were recorded on a Nicolet Impact 400 FT-IR spectrometer. ¹H and ¹³C NMR spectra were recorded on a Bruker AM500 (500 MHz) spectrometer. ¹H-¹H COSY, HMQC and NOESY experiments were performed on a Bruker DMX-500 spectrometer. Chemical shifts for proton and carbon resonances are reported in ppm (δ) relative to chloroform (δ 7.26, 77.07 respectively). X-ray data were collected on a Bruker Siemens SMART CCD (charge coupled device) based diffractometer equipped with an LT-2 low-temperature aparatus operating at 213K.

Data for Trimethyl [(E)-oct-1-enyl]stannane (9)⁶

¹H NMR (500 MHz, CDCl₃) δ 5.91-6.00 (m, 2H), 2.10-2.14 (m, 2H), 1.36-1.40 (m, 2H), 1.28-1.32 (m, 6H), 0.88 (t, 3H, J=6.9 Hz), 0.10 (s, 9H); ¹³C NMR (125 MHz, CDCl₃) δ 149.6, 127.8, 37.7, 31.8, 29.0, 28.8, 22.7, 14.1, −9.7

Data for Methyl 1-[(E)-oct-1-enyl]-2-oxocyclopentanecarboxylate (10)⁷

¹H NMR (500 MHz, CDCl₃) δ 5.54-5.62 (m, 2H), 3.70 (s, 3H), 2.57 (dt, 1H, J₁=13.2 Hz, J₂=7.3 Hz), 2.27-2.40 (m, 2H), 2.14 (qt, 1H, J=6.4 Hz), 2.03-2.07 (m, 2H), 1.94-2.01 (m, 1H), 1.87-1.93 (m, 1H), 1.32-1.37 (m, 2H), 1.21-1.29 (m, 6H), 0.86 (t, 3H, J=7.0 Hz); ¹³C NMR (125 MHz, CDCl₃) δ 212.8, 171.2, 133.6, 125.8, 62.7, 52.8, 37.5, 33.5, 33.5, 32.7, 31.6, 29.0, 28.8, 22.6, 19.5, 14.1; FTIR (neat, cm⁻¹) 1753 (s, C═O), 1739 (s, C═O); MS (EI) 252.

Preparation of cis-1-propenylmagnesium bromide (11)

A three-necked, 100-mL round-bottomed flask equipped with a condensor and an addition funnel was charged with magnesium powder (0.24 g, 10 mmol, 1.0 equiv) and tetrahydrofuran (2 mL). Seven drops of cis-1-bromopropene (0.85 mL, 10 mmol, 1.0 equiv) in tetrahydrofuran (18 mL) was added by addition funnel. After ca. 5 min, the Grignard reaction initiated and the remaining bromide solution was added dropwise by addition funnel slowly. This solution was stirred at room temperature for 2 h to complete the reaction.

Preparation of trans-1-propenylmagnesium bromide (X)

Neat 1,2-dibromoethane (0.51 mL, 5.7 mmol, 1.0 equiv) was added to a suspension of magnesium powder (0.146 g, 6.0 mmol, 1.05 equiv), ether (4.3 mL), and benzene (1.4 mL) over 10 min, and the resulting solution was stirred at 40° C. for 1 h to provide anhydrous magnesium bromide. t-Butyl lithium (1.7 M, 6.7 mL, 11.4 mmol, 2 equiv) was added dropwise over 5 min to a 100-mL Schlenk flask containing trans-1-bromopropene (0.49 mL, 5.7 mmol, 1 equiv), tetrahydrofuran (16 mL), ether (4 mL), and pentane (4 mL) at −130° C. (pentane/liquid nitrogen). The resulting yellow solution was maintained below −110° C. for 1 h, then warmed to −78° C. The freshly prepared magnesium bromide was added to the vinyl lithium and stirred for 30 min at −78° C. Upon warming to room temperature, approximately 75% of the solvent was removed in vacuo, tetrahydrofuran (10 mL) was added, and approximately half of the solvent was removed again.

Synthesis of (Z)-(7R *,8S*)-8-hexyl-7-methylbicyclo[4.3.1]deca-1(9)-en-5,10-dione (13)

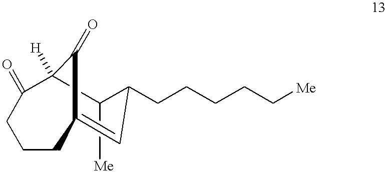

A solution of cis-l-propenyl-magnesium bromide 11 in tetrahydrofuran (0.49 M, 3.7 n, 1.8 mmol, 2.1 equiv) was added to a suspension of β-ketoester 10 (0.214 g, 0.85 mmol, 1 equiv) and 4 Å molecular sieves (0.79 g) in tetrahydrofuran (8 mL) at −78° C. The suspension was stirred at −78° C. for 1.5 h, at 0° C. for 1 h, and at room temperature for 12 h. The mixture was quenched by the addition of glacial acetic acid (0.12 mL, 1.8 mmol, 2.1 equiv) and stirred for 10 min. The mixture was filtered through a pad of Celite, washed extensively with ether (100 mL) and then concentrated. Purification of the residue by flash column chromatography eluting with a gradient of dichloromethane-hexane (50→70→100%) afforded (Z)-(7R*,8S*)-8-hexyl-7-methylbicyclo[4.3.1]deca-1(9)-en-5,10-dione (13) (0.145 g, 65%) as a colorless oil. Also observed was methyl (Z)-(3*,175*)-3-hexyl-4-methyl-6-oxocyclonona-1-en-1-carboxylate (18) in a ratio of 1:9 (18:13).

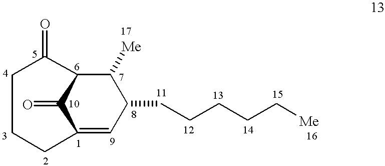

¹H NMR (500 MHz, CDCl₃) δ 5.95 (d, 1H, J=4.2 Hz, H₉), 3.43 (d, 1H, J=8.8 Hz, H₆), 2.69-2.75 (m, 1H, H₂), 2.54-2.61 (m, 1H, H₇), 2.47-2.52 (m, 1H, H₄), 2.39-2.45 (m, 1H, H_{4′}), 2.25 (dt, 1H, J₁=12.8 Hz, J₂=7.6 Hz, H_{2′}), 2.14-2.19 (m, 1H, H₈), 1.75-1.85 (m, 2H, H₃), 1.41-1.46 (m, 2H, H₁₁), 1.33-1.39 (m, 1H, H₁₂), 1.23-1.29 (m, 7H, H_{12′-15}), 0.97 (d, 3H, J=7.5 Hz, H₁₇), 0.86 (t, 3H, J=6.9 Hz, H₁₆); ¹³C NMR (125 MHz, CDCl₃) δ 207.3, 206.5, 142.4 (C₁), 136.1 (C₉), 68.8 (C₆), 45.1 (C₄), 38.0 (C₈), 36.1 (C₇), 31.8, 31.1 (C₁₁), 31.0 (C₂), 29.3, 28.0, 25.1 (C₃), 22.6, 14.1 (C₁₆), 13.2 (C₁₇); FTIR (neat, cm⁻¹) 1726 (s, C═O), 1699 (s, C═O); HRMS (EI) calcd for C₁₇H₂₆O₂(M)⁺ 262.1933, found 262.1924.

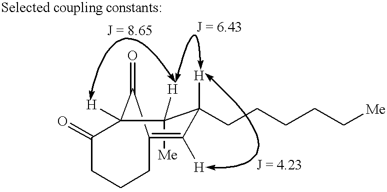

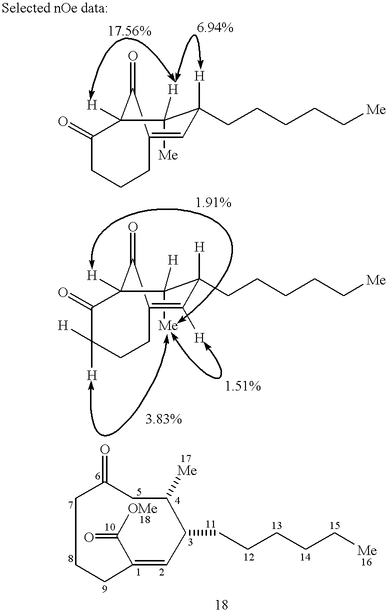

¹H NMR (500 MHz, CDCl₃) δ 5.88 (d, 1H, J=8.6 Hz, H₂), 3.69 (s, 3H, H₁₈), 3.16-3.22 (m, 1H, H₃), 2.69-2.75 (m, 2H, H_{4, 7}), 2.47 (dd, 1H, J₁=12.7 Hz, J₂=11.0 Hz, H₅), 2.21-2.32 (m, 3H, H_{8, 9}), 1.85-1.90 (m, 1H, H_{8′}), 1.75-1.80 (m, 2H, H_{5′, 7′}), 1.47-1.51 (m, 1H, H₁₂), 1.24-1.29 (m, 7H, H_{12′-15}), 1.08-1.16 (m, 2H, H₁₁), 1.00 (d, 3H, J=7.3 Hz, H₁₇), 0.85 (t, 3H, J=6.9 Hz, H₁₆); ¹³C NMR (125 MHz, CDCl₃) δ 214.5 (C₆), 166.2 (C₁₀), 153.0 (C₂), 133.5 (C₁), 51.4 (C₁₈), 47.8 (C₅), 42.3 (C₉), 40.5 (C₃), 38.8 (C₄), 34.6 (C₇), 31.9, 29.5, 28.8 (C₈), 28.4 (C₁₂), 28.0 (C₁₁), 22.6, 19.8 (C₁₇), 14.1 (C₁₆); FTIR (neat, cm⁻¹) 1726 (s, C═O), 1699 (s, C═O); HRMS (EI) calcd for C₁₈H₃₀O₃(M)⁺ 294.2195, found 294.2197. X-ray Crystallography confirms this structure.

Synthesis of (Z)-(7S*,8S*)-8-hexyl-7-methylbicyclo[4.3.1)deca-1(9)-en-5,10-dione (14).

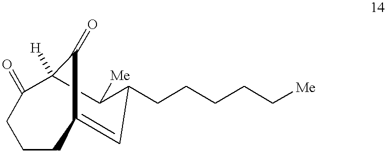

A solution of trans-1-propenyl-magnesium bromide 12 in tetrahydrofuran (0.66 M, 3.5 mL, 2.3 mmol, 1.5 equiv) was added to a suspension of β-ketoester 10 (0.386 g, 1.53 mmol, 1.0 equiv) and 4 Å molecular sieves (0.99 g) in toluene (13 mL) at −78° C. The suspension was stirred at −78° C. for 1.5 h and benzophenone (0.267 g, 1.5 mmol, 0.84 equiv) in tetrahydrofuran (2 mL) was added to quench the unreacted 12. The suspension was then stirred at 0° C. for 1 h, and at room temperature for 14 h. The mixture was quenched by the addition of glacial acetic acid (0.20 mL, 3.0 mmol, 2.0 equiv) and stirred for 20 min. The mixture was poured into a 1:1 aqueous solution of saturated sodium chloride and saturated ammonium chloride (40 mL), washed extensively with ether (3×50 mL), dried with magnesium sulfate, and concentrated. Purification of the residue by flash column chromatography (5% ethyl acetate-hexane) afforded (Z)-(7S*,g8*)-8-hexyl-7-methylbicyclo[4.3.1]deca-1(9)-en-5,10-dione (14) (0.119 g, 30%) as a colorless oil. Also observed was methyl (Z)-(3S*,17R*)-3-hexyl-4-methyl-6-oxocyclonona-1-en-1-carboxylate (19), methyl (E)-(3S*,17R *)-3-hexyl4-methyl-6-oxocyclonona-1-en-1-carboxylate (20), and methyl (E)-(3R*,17R*)-3-hexyl-4-methyl-6-oxocyclonona-1-en-1-carboxylate (21) in a ratio of 3:2:1:9 (19:20:21:14). Quenching the reaction at −78° C. affords methyl (1R*,2R*)-1-[(E)oct-1-enyl]-2-[(E)-prop-1-enyl]-cyclopenta-2-ol-carboxylate (22).

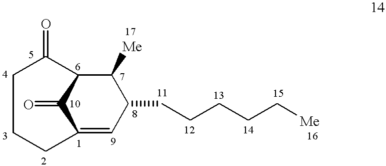

¹H NMR (500 MHz, CDCl₃) δ 5.80 (d, 1H, J=2.3 Hz, H₉), 2.82 (s, 1H, H₆), 2.51-2.62 (m, 2H, H_{2, 4}), 2.41 (qt, 1H, J=6.9 Hz, H₇), 2.32-2.37 (m, 2H, H_{2′, 4′}), 2.07-2.12 (m, 1H, H₃), 1.64-1.73 (m, 1H, H_{3′}), 1.57-1.61 (m, 1H, H₈), 1.53-1.56 (m, 1H, H₁₁), 1.40-1.44 (m, 1H, H_{11′}), 1.27-1.33 (m, 8H, H₁₂₋₁₅), 1.10 (d, 3H, J=7.0 Hz, H₁₇), 0.88 (t, 3H, J=6.8 Hz, H₁₆); ¹³C NMR (125 MHz, CDCl₃) 208.1, 207.8, 145.3 (C₁), 135.9 (C₉), 69.8 (C₆), 43.0 (C₄), 40.2 (C₈), 35.4 (C₇), 34.14 (C₃), 34.08 (C₁₁), 31.7, 31.5 (C₂), 29.3, 27.3, 22.6, 21.2 (C₁₇), 14.1 (C₁₆); FTIR (neat, cm⁻¹) 1737 (s, C═O), 1705 (s, C═O); HRMS (EI) calcd for C₁₇H₂₆O₂(M)⁺ 262.1933, found 262.1944.

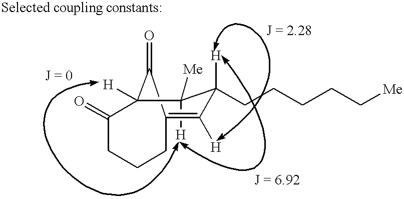

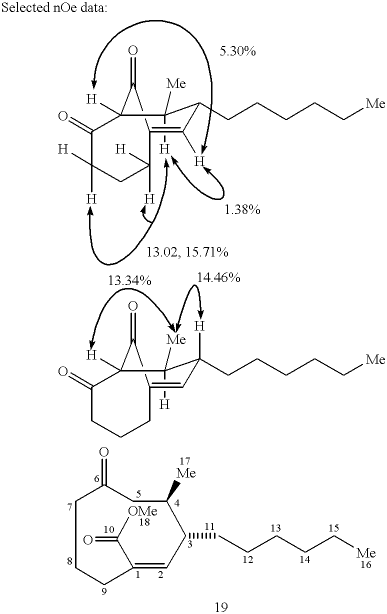

¹H NMR (500 MHz, CDCl₃) δ 5.39 (dd, 1H, J₁=11.7 Hz, J₂=0.7 Hz, H₂), 3.77 (s, 3H, H₁₈), 2.67-2.73 (m, 1H, H₃), 2.48-2.55 (m, 1H, H₉), 2.35-2.40 (m, 2H, H_{5, 7}), 2.11-2.24 (m, 4H, H_{7′, 8′, 9′}), 2.00-2.08 (m, 2H, H_{4, 5′}), 1.64-1.70 (m, 1H, H₁₁), 1.18-1.32 (m, 8H, H₁₂₋₁₅) 1.09-1.16 (m, 1H, H_{11′}), 1.05 (d, 3H, J=6.7 Hz, H₁₇), 0.86 (t, 3H, J=7.0 Hz, H₁₆); ¹³C NMR (125 MHz, CDCl₃) δ 215.3 (C₆), 169.4 (C₁₀), 151.8 (C₂), 133.3 (C₁), 51.7 (C₅), 51.6 (C₁₈), 47.1 (C₃), 41.9 (C₇), 40.5 (C₄), 32.9 (C₁₁), 31.8, 30.0 (C₉), 29.5, 28.3, 28.2 (C₈), 22.7, 20.1 (C₁₇), 14.1 (C₁₆); FTIR (neat, cm⁻¹) 1723 (s, C═O), 1703 (s, C═O); HRMS (CI) calcd for C₁₈H₃₄NO₃(M+NH₄)⁺ 312.2539, found 312.2534.

¹H NMR (500 MHz, CDCl₃) δ 6.55 (d, 1H, J=2.4 Hz, H₂), 3.65 (s, 3H, H₁₈), 2.46 (dd, 1H, J₁=16.2 Hz, J₂=4.0 Hz, H₅), 2.29 (t, 2H, J=7.5 Hz, H_{7 or 9}), 2.20 (t, 2H, J=7.6 Hz, H_{7 or 9}), 2.12 (dd, 1H, J₁=16.2 Hz, J₂=11.6 Hz, H_{5′}), 2.08-2.04 (m, 1H, H₃), 1.97-1.90 (m, 1H, H₄), 1.73 (qt, 1H, J=−7.6 Hz, H₇), 1.63-1.56 (m, 1H, H₁₁), 1.46-1.38 (m, 2H, H_{11′,12}), 1.34-1.21 (m, 7H, H_{12′-15}), 1.02 (d, 3H, J=6.6 Hz, H₁₇), 0.89 (t, 3H, J=6.7 Hz, H₁₆); ¹³C NMR (125 MHz, CDCl₃) δ 199.6 (C₆), 174.0 (C₁₀), 149.8 (C₂), 137.9 (C₁), 51.5 (C₁₈), 45.7 (C₅), 43.5 (C₃), 34.2 (C₄), 33.6 (C_{7 or 9}), 32.1 (C₁₁), 31.8, 29.6, 28.8 (C_{7 or 9}), 26.3 (C₁₂), 23.9 (C₈), 22.7, 19.5 (C₁₇), 14.1 (C₁₆); FTIR (neat, cm⁻¹) 1736 (s, C═O), 1677 (s, C═O); HRMS (EI) calcd for C₁₈H₃₀O₃(M)⁺ 294.2195, found 294.2192.

¹H NMR (500 MHz, CDCl₃) δ 6.66 (d, 1H, J=11.6 Hz, H₂), 3.76 (s, 3H, H₁₈), 2.55-2.38 (m, 5H), 2.28-2.23 (m, 1H), 2.20-2.10 (m, 3H), 1.91-1.84 (m, 1H), 1.43-1.32 (m, 2H, H₁₁), 1.28-1.20 (m, 6H, H₁₃₋₁₅), 1.14-1.09 (m, 2H, H₁₂), 0.92 (d, 3H, J=6.5 Hz, H₁₇), 0.85 (t, 3H, J=7.0 Hz, H₁₆); ¹³C NMR (125 MHz, CDCl₃) δ 216.3 (C₆), 167.9 (C₁₀), 144.3 (C₂), 133.6 (C₁), 51.9 (C₁₈), 48.6, 43.4, 39.7 (C₃), 36.0 (C₄), 33.1, 31.8, 29.4, 27.9, 24.4 (C₅), 24.0, 22.6, 14.9 (C₁₇), 14.1 (C₁₆); FTIR (neat, cm⁻¹) 1721 (s, C═O), 1698 (s, C═O); HRMS (EI) calcd for C₁₈H₃₀O₃(M)⁺ 294.2195, found 294.2185.

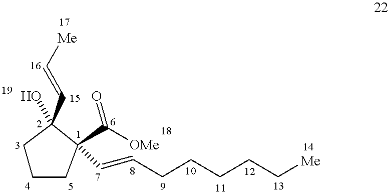

¹H NMR (500 MHz, CDCl₃) δ 5.80 (dt, 1H, J₁=15.8 Hz, J₂=1.2 Hz, H₇), 5.72 (dq, 1H, J₁=15.4 Hz, J₂=6.4 Hz, H₁₆),5.64 (dt, 1H, J₁=15.8 Hz, J₂=6.8 Hz, H₈), 5.57 (dq, 1H, J₁=15.4 Hz, J₂=1.5 Hz, H₁₅), 3.65 (s, 3H, H₁₈), 2.17-2.04 (m, 4H, H₉), 1.95-1.74 (m, 4H, H), 1.70 (dd, 3H, J₁=6.5 Hz, J₂=1.5 Hz, H₁₇), 1.37 (qt, 2H, J=1.2 Hz, H₁₀), 1.31-1.22 (m, 7H, H_{11-13, 19}), 0.86 (t, 3H, J=6.9 Hz, H₁₄); ¹³C NMR (125 MHz, CDCl₃) δ 174.7, 133.6, 133.4, 127.4, 124.9, 83.1, 62.7, 51.8, 36.0, 33.0, 31.7, 29.8, 29.3, 28.8, 22.7, 19.2, 18.0, 14.1; FTIR (neat, cm⁻¹) 1729 (s, C═O).

What we claim is:

1. A method for generating a library of isolated biomimetic compounds comprising:
selecting a desired biomimetic synthetic pathway;
recreating said selected biomimetic synthetic pathway using appropriate synthetic reagents to yield a diversifiable biomimetic structure;
diversifying said biomimetic structure to yield a library of biomimetic compounds.

2. The method of claim 1, wherein said diversifiable biomimetic structure is generated in fewer than four steps.

3. The method of claim 1, further comprising attachment of at least one of said available synthetic reagents or said diversifiable biomimetic structure to a solid support unit.

4. The method of claim 1, wherein recreating said selected biomimetic synthetic pathway comprises utilizing an existing biomimetic synthetic pathway.

5. The method of claim 1, wherein recreating said selected biomimetic synthetic pathway comprises modifying an existing biomimetic synthetic pathway to achieve different reactivity.

6. A method for generating a library of isolated biomimetic compounds using an oxidative phenolic coupling reaction comprising:
providing a first phenol comprising the following structure: 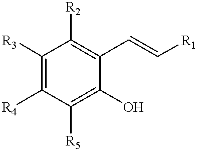
wherein R₁, R₂, R₄, and R₅, as valency and stability permit, are each independently selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower aLkylamino, nitro, phenoxy, benzyloxy, hydrogen, or any derivative incorporating phosphorous; wherein R₃ is an electron withdrawing group, or any of R₂, R₃, R₄, and R₅ taken together form a carbocycle or heterocycle having from 3 to 10 atoms in the ring,;
providing a second phenol comprising the following structure: 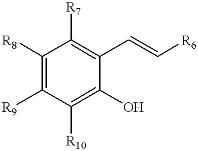
wherein R₆, R₇, R₉, and R₁₀ as valency and stability permit, are each independently selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocyclyl is substituted with 1-5 substituents selected from the group consisting of lower alkyl, halo,.hydroxy, amino, thio, lower alkoxy, lower alkylthio, lower alkylamino, nitro, phenoxy, benzyloxy, or hydrogen, any derivative incorporating phosphorous; wherein R₈ is an electron donating group, or R₇, R₈, R₉ and R₁₀ taken together form a carbocycle or heterocycle having from 3 to 10 atoms in the ring,
reacting the phenols to yield a complex scaffold structure comprising the following structure: 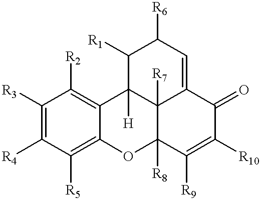
wherein R₁-R₁₀, as valency and stability permit, are each independently selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulflhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, and any derivative incorporating phosphorous; or wherein either any of R₂, R₃, R₄, and R₅ taken together, or any of R₇, R₈, and R₉ taken together form a carbocycle or heterocycle having from 3 to 10 atoms in the ring;
diversifying said complex scaffold structure at desired functional moieties to yield a library of complex natural product-like compounds.

7. The method of claim 6, wherein one of said phenols is attached to a solid support.

8. The method of claim 6, wherein said first and second phenols are identical and the oxidative phenolic coupling reaction comprises a homocoupling reaction.

9. The method of claim 6, wherein said first phenol comprises an electron deficient phenol and said second phenol comprises an electron rich phenol and the oxidative phenolic coupling reaction comprises a heterocoupling reaction.

10. A method for the synthesis of isolated biomimetic scaffold structures using an intramolecular oxidative phenolic coupling reaction comprising:
providing two linked phenols having the following structure: 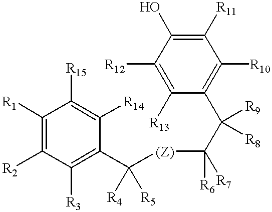
wherein R₁-R₁₅, as valency and stability permit, are each independently selected from the group consisting of a linear or branched alkyl, alkenyl, linear or branched arninoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, and any derivative incorporating phosphorous wherein any of R₁,-R₁₅ taken together, as chemically permissible, form a carbocycle or heterocycle having from 3 to 10 atoms in the ring, and wherein at least one of R₁, R₂, R₃, or R₁₅ comprises a phenolic substrate.
reacting said linked phenols to yield a biomimetic scaffold structure;
diversifying said complex scaffold structure at desired functional moieties to yield a library of isolated biomimetic compounds.

11. The method of claim 10, wherein said reaction occurs via a para-ortho oxidative phenolic coupling and said biomimetic scaffold comprises the following structure: 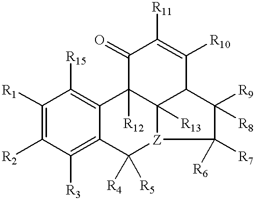
wherein R₁-R₁₅, as valence and stability permit, are each independently selected from the group consisting of a linear or branched alkyl, alkenyl, linear or branched atnino alkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thiio, aryloxy, arylalkoxy, hydrogen, alkynyl, halogen, cyano, sulthydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amnino, thio, lower alkoxy, lower alkylthio, lower alkylamino, nitro, phenoxy, benzyloxy, hydrogen, and any derivative incorporating phosphorous; or wherein any of R₁,-R₁₅ taken together, as chemically permissible, formn a carbo cycle or heterocycle having from 3 to 10 atoms in the ring; and wherein Z is a carbon, nitrogen, sulflar, or oxygen functionality.

12. The method of claim 10, wherein said reaction occurs via a para-para oxidative phenolic coupling and said biomimetic scaffold comprises the following structure: 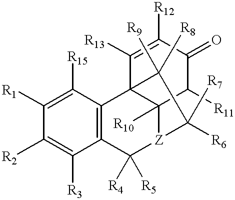
wherein R₁-R₁₅, as valence and stability permit, are each independently selected from the group consisting of a linear or branched alkyl, alkenyl, linear or branched aminoalkyl, linear or branched acylamino, linear or branched ayloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylarnino, nitro, phenoxy, benzyloxy, hydrogen, and any derivative incorporating phosphorous; or wherein any of R₁,-R₁₅,taken together form a carbocycle or heterocycle having from 3 to 10 atoms in the ring; and wherein Z is a carbon, oxygen, nitrogen or sulfur functionality.

13. The method of claim 10, wherein said reaction occurs via a ortho-para oxidative phenolic coupling and said biomimetic scaffold comprises the following structure: 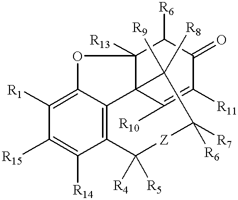
wherein R₁-R₁₅, as valence and stability permit, are each independently selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, and any derivative incorporating phosphorous; or wherein any of R₁-R₁₅, taken together, as chemically permissible, form a carbocycle or heterocycle having from 3 to 10 atoms in the ring; and wherein Z is an oxygen, sulfur, nitrogen, or carbon functionality.

14. A library of isolated biomimetic compounds having the following structure: 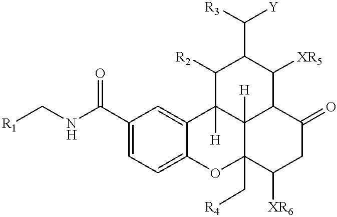
wherein R₁-R₆, as valency and stability permit, are each independently selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulffhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, and any derivative incorporating phosphorous; wherein Y is any of the above or a linking unit; and X is any sulfur, oxygen, nitrogen, phosphorous, or carbon functionality.

15. A library of isolated biomimetic compounds having the following structure: 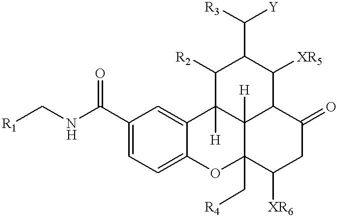
wherein R₁-R₆, as valency and stability permit, are each independently selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, and any derivative incorporating phosphorous; wherein Y is any of the above or a linking unit; and X is any sulfur, oxygen, nitrogen, phosphorous, or carbon functionality,
wherein said library is produced by the method of claim 6.

16. A library of isolated biomimetic compounds comprising the following structure: 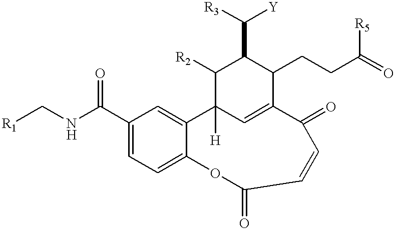
wherein R₁-R₃ and R₅, as valency and stability permit, are each independently selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, and any derivative incorporating phosphorous; and wherein Y is any of the above, a linking unit, or a biomolecule.

17. A library of isolated biomimetic compounds comprising the following structure: 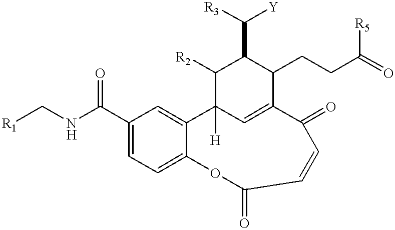
wherein R₁-R₃ and R₅ , as valency and stability permit, are each independently selected from the group consisting of a linear or branched alkyl, alkenyl, linear or branched arninoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulffhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, and any derivative incorporating phosphorous; and wherein Y is any of the above, a linking unit, or a biomolecule,
wherein said library is produced by the method of claim 6.

18. A library of biomimetic compounds comprising the following structure: 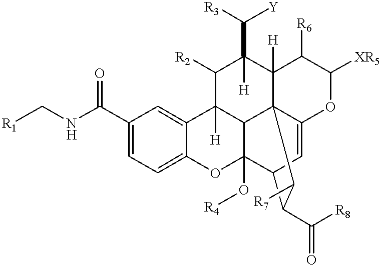
wherein R₁-R₈, as valency and stability permit, are each independently selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, and any derivative incorporating phosphorous; wherein Y is any of the above, a linking unit, or a biomolecule; and X is any sulfur, oxygen, nitrogen, phosphorous, or carbon functionality.

19. A library of biomimetic compounds comprising the following structure: 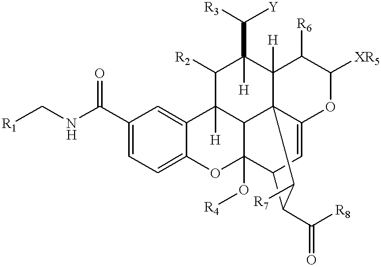
wherein R₁-R₈, as valency and stability pennit, are each independently selected from the group consisting of a linear or branched alkyl, alkenyl, linear or branched amino alkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, allyyl, halogen, cyano, sulfhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylarnino, nitro, phenoxy, benzyloxy, hydrogen, and any derivative incorporating phosphorous; wherein Y is any of the above, a linking unit, or a biornolecule; and X is any sulfur, oxygen, nitrogen, phosphorous, or carbon functionality, wherein said library is produced by the method in claim 6.

20. A library of biomimetic compounds comprising the following structure: 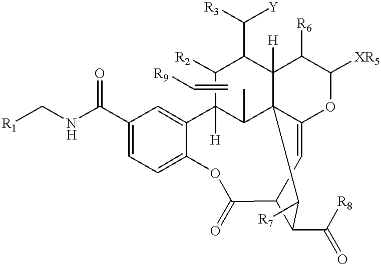
wherein R₁-R₉, as valency and stability permit are each independently selected from the group consisting of a linear or branched alkyl, alkenyl, linear or branched amnino alkyl, linear or branched acylamino, linear or branched acyloxy, linear or branched alkoxycarbonyl, linear or branched alkoxy, lin ear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, and any derivative incorporating phosphorous; wherein Y is any of the above, a linking unit or a biomolecule; and X is any sulfur, oxygen, nitrogen, phosphorous, or carbon functionality.

21. A library of biomimetic compounds comprising the following structure: 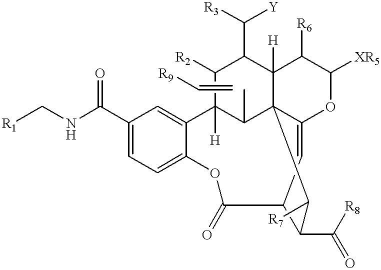
wherein R₁-R₉, as valency and stability permit, are each independently selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, arnino, hydroxy, thio, aryloxy, arylalkoxy, hydrogen, alkynyl, halogen, cyano, sulffhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, and any derivative incorporating phosphorous; wherein Y is any of the above, a linking unit or a biomolecule; and X is any sulfur, oxygen, nitrogen, phosphorous, or carbon functionality, wherein said library is produced by the method of claim 6.

22. A method for generating a library of isolated biomimetic compounds comprising:
synthesizing a vinyl stannane from a substituted alkyne;
reacting a cyclic β-keto ester with said vinyl stannane under conditions to generate a 2-vinyl-2-methoxycycloalkanone;
reacting said cycloalkanone with a Grignard reagent to generate a biomimetic scaffold bicyclo [n.3.1] ring system;
diversifying said biomimetic scaffold bicyclo [n.3.1] ring system at selected reactive moieties to generate a library of biomimetic bicyclo [n.3. 1] ring system compounds.

23. A library of isolated biomimetic compounds having the following structure: 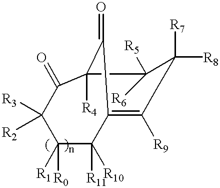
wherein R₀-R₁₁, as valency and stability permit, are each independently selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, and any derivative incorporating phosphorous; wherein either any of R₀-R₁₁ taken together form a carbocycle or heterocycle having from 3 to 10 atoms in the ring, and wherein n is 0 to 3.

24. A method for generating a library of isolated biomimetic compounds comprising:
synthesizing a vinyl stannane from a substituted alkyne;
vinylating a cyclic β-keto ester to generate a 2-vinyl-2-methoxycycloalkanone;
reacting said cycloalkanone with a vinyl Grignard reagent, and trapping with an electrophile to generate diversifiable medium ring structures;
diversifying said biomimetic medium ring structures at selected reactive moieties to generate a library of biomimetic medium ring structures.

25. A library of isolated biomimetic medium ring structures comprising the following structure: 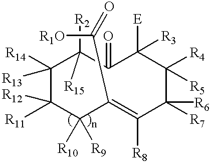
wherein R₁-R₁₅, as valency and stability permit, are each independently selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulihydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, and any derivative incorporating phosphorous; or wherein either any of R₁-R₁₅ taken together form a carbocycle or heterocycle having from 3 to 10 atoms in the ring; wherein E is a functionality resulting from reaction with an electrophile; and wherein n is 0-3.

26. The library of claim 25, wherein any carbon atom in the biomimetic skeleton is substituted with a nitrogen, oxygen or sulfur atom.

27. A method for the generation of a library of isolated fused medium ring structures comprising;
synthesizing a medium ring structure by the method of claim 24;
reacting said medium ring structures with a base to, and subsequent trapping with an electrophile to generate a diversifiable biomimetic scaffold fused medium ring structure;
functionalizing said biomimetic medium ring structure to generate a library of biomimetic fused medium ring compounds.

28. A library of biomimetic fused medium ring structures having the following structure: 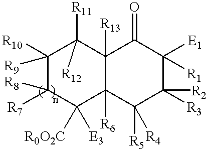
wherein R₀-R₁₃, as valency and stability permit, are each independently selected from the group consisting of a linear or branched alkyl, alkenyl, linear or branched aminoalkyl, linear or branched acylamino, linear or branched acyloxy, linear or branched alkoxycarbonyl, linear or branched alkoxy, linear or branched alkylaryl, linear or branched hyrdoxyalkyl, linear or branched thioalkyl, acyl, amino, hydroxy, thio, aryloxy, arylalkoxy, hydrogen, alkynyl, halogen, cyano, sulfhydryl, carbamoyl, nitro, trifluoromethyl, and substituted or unsubstituted heterocyclyl, wherein said heterocyclyl is substituted with 1-5 substituents selected from the group consisting of lower alkyl, halo, hydroxy, amino, thio, lower alkoxy, lower alkylthio, lower alkylamino, nitro, phenoxy, benzyloxy, hydrogen, and any derivative incorporating phosphorous; or wherein any of R₀-R₁₃ taken together form a carbocycle or heterocycle having from 0 to 10 atoms in the ring; wherein E₁ and E₃ are functionalities resulting from reaction with electrophiles; and wherein n is 0-3.

29. A pharmaceutical composition comprising:
a biomimetic library member; and
a pharmaceutically acceptable composition.

30. A kit for determining one or more biological activities of biomimetic library members comprising:
a library of biomimetic compounds; and
a reagent for determining one or more biological activities of said biomimetic compounds.

31. A method for determining one or more biological activities of biomimetic library members comprising:
providing a library of biomimetic compounds;
subjecting the library of biomimetic compounds to a biological target;
determining a statistically significant change in a biochemical activity relative to the level of biochemical activity in the absence of the compound.
